(19) United States
(12) Patent Application Publication
(10) Pub. No.: US 2018/0118707 A1
(43) Pub. Date: May 3, 2018

(54) BETA-NAPHTHOISOFLAVONES, COMPOSITIONS CONTAINING, AND USES OF, SAME

(71) Applicant: Thesan Pharmaceuticals, Inc., Carlsbad (CA)

(72) Inventors: Nigel Robert Arnold Beeley, Solana Beach, CA (US); Howard Glenn Welgus, Ballwin, MO (US); Jay Edward Birnbaum, Montville, NJ (US); J. Gordon Foulkes, Rancho Santa Fe, CA (US); Celia Patricia Jenkinson, San Diego, CA (US); Jean Hilaire Saurat, Geneva (CH)

(73) Assignee: Thesan Pharmaceuticals, Inc., Carlsbad, CA (US)

(21) Appl. No.: 15/735,053

(22) PCT Filed: Jun. 7, 2016
(86) PCT No.: PCT/US2016/036226

§ 371 Date: Dec. 8, 2017

Related U.S. Application Data
(60) Provisional application No. 62/173,102, filed on Jun. 9, 2015.

(51) Int. Cl. C07D 311/80; A61P 17/00; A61K 9/00; A61P 17/08; A61P 17/10; A61P 23/02; A61P 29/00; A61P 19/02; A61P 1/04; A61P 19/04; A61P 17/06; A61P 11/06; A61P 37/06; A61P 25/28; A61P 25/00; A61P 3/00; A61P 3/04; A61K 31/352; C07D 407/04; A61K 31/357; C07D 493/04; C07D 405/04; A61K 31/4433; C07D 491/056; A61K 31/4355; A61K 31/436

(52) U.S. Cl. CPC C07D 311/80; C07B 2200/09; A61K 9/0014; A61P 17/08; A61P 17/10; A61P 23/02; A61P 29/00; A61P 19/02; A61P 1/04; A61P 19/04; A61P 17/06; A61P 11/06; A61P 37/06; A61P 25/28; A61P 25/00; A61P 3/00; A61P 3/04; A61K 31/352; C07D 407/04; A61K 31/357; C07D 493/04; C07D 405/04; A61K 31/4433; C07D 491/056; A61K 31/4355; A61K 31/436; C07B 2200/07; A61P 17/00

(57) ABSTRACT

The present invention provides compounds of the following structure,

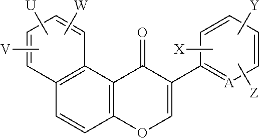

methods of using such compounds, and pharmaceutical compositions containing such compounds. In addition, this invention provides methods for the treatment and/or prevention of disease states mediated by Aryl Hydrocarbon receptor pathways.

### FIELD OF THE INVENTION

The present invention relates to compounds having the structure I and to pharmaceutical compositions and uses of such compounds as lipid modulators, as activators of Aryl Hydrocarbon receptor pathways and for the treatment of a disease, diseases, health care or cosmetic problems.

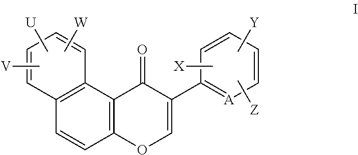

In particular the present invention relates to the treatment and/or prevention of a skin condition associated with abnormal sebum secretion or abnormal sebaceous gland function. The present invention relates more specifically to pharmaceutical compositions for topical use comprising a compound of structure I having improved pharmaceutical properties over 3-phenyl-1H-benzo[f]chromen-1-one. Local application of such pharmaceutical compositions effectively treats skin conditions including, but not limited to, excess sebum production, acne, oily skin, oily hair, shiny or greasy-looking skin, hyper-seborrhea, seborrheic dermatitis, rosacea, sebaceous hyperplasia, and sebaceous carcinoma. These compounds and compositions may also be useful for fat reduction from areas such as lipomas and submental fat, and for body sculpting.

### BACKGROUND TO THE INVENTION

3-Phenyl-1H-benzo[f]chromen-1-one modulates the synthesis of multiple enzymes involved in lipid metabolism as shown in PCT International Application Publication No. WO/2013/171696. Without wishing to be bound by a particular biochemical theory, one possible mechanism for these effects is the activation of a transcription factor called the aryl-hydrocarbon receptor which leads to down-regulation, or decreased expression, of genes for lipid-modifying enzymes (See, also, PCI International Application Publication No. WO/2009/093207). However, the structure of 3-phenyl-1H-benzo[f]chromen-1-one was not designed for modulating lipid synthesis and/or lipid secretion in viva and has not been optimized for potency at any receptor or target, or for bioavailability, pharmacokinetic or solubility properties in appropriate pharmaceutical formulations.

Acne is the most common skin disease. It has a high impact on quality of life and is associated with depression, anxiety, and loss of self-esteem. Of all skin diseases, acne entails direct medical costs second only in magnitude to skin ulcers and wounds. Acne often appears at the onset of puberty, and its prevalence is highest in the middle to late teenage population, although it can persist into middle age, especially in women (Zaenglein (2012)). The overall population prevalence has been estimated at 14% (Tan (2008)). Several treatments already exist for this and related conditions of the skin, but none of them are without significant drawbacks. Thus, by way of example, it is known that a vitamin A derivative, isotretinoin (otherwise known as 13-cis-retinoic acid, Accutanefl, is the most efficacious drug in the treatment of severe acne, and acts by inducing atrophy of the sebaceous glands with consequent sebum reduction. However, this substance must be administered systemically to maximize efficacy, since topical administration does not cause sebum reduction. However, such systemic administration causes significant unwanted side effects. Notably, oral isotretinoin is a known severe teratogen, with the potential to cause birth defects due to in utero exposure. It is now only available in the USA after other acne treatments have failed and under a special prescription program where patients use multiple forms of birth control. Thus, although this drug is highly effective, safety concerns and overall benefit vs. risk considerations preclude its use during the earlier stages of acne and often only after the appearance of severe and often disfiguring scars.

The pathogenesis of acne involves several elements including excess sebum production, follicular epidermal hyper-proliferation, inflammation, and the presence of the bacterium Propionibacterium acnes. Studies have shown a strong correlation between the sebum excretion rate (SER) and untreated acne severity. People with low or normal SER do not get acne or have very mild forms, whereas people with high SER are more acne-prone, and the higher the SER, the more severe the acne. In addition, the reduction in SER produced by systemically administered drugs correlates directly with objective acne improvement measures (Janiczek-Dolphin (2010)). Topical application of 3-phenyl-1H-benzo[f]chromen-1-one can reduce sebum production and inflammation and analogs thereof represent novel drugs for the treatment of acne.

Acne represents only one example of the potential therapeutic utility of 3-phenyl-1H-benzo[f]chromen-1-one analogs. Related skin conditions include oily skin, oily hair, shiny or greasy-looking skin, acne, rosacea, hyperseborrhea, seborrheic dermatitis, sebaceous hyperplasia, and sebaceous carcinoma. Regulation of some of the same biochemical pathways as in the sebaceous glands can also occur in adipose tissue, so yet other applications involve the potential diminution and/or removal of fat cells in conditions such as lipomas, and excess submental fat. The analogs may also be useful for body sculpting.

There remains, therefore, an unmet need to develop new medicaments against the conditions mentioned above, and in particular pharmaceutical compositions for topical use which make it possible to avoid the drawbacks associated with systemic administration.

### SUMMARY OF THE INVENTION

The subject invention provides a method of treating a skin condition associated with abnormal sebum secretion or abnormal sebaceous gland function in a subject which comprises topically and periodically applying to an area of subject's skin affected by the skin condition a composition comprising a pharmaceutically acceptable carrier and an amount of a compound or of a pharmaceutically acceptable salt of the compound effective to treat the skin condition, wherein the compound has the structure:

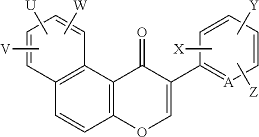

wherein:

each of U, V, W, X, Y, and Z is independently:

H; OH; F; Cl; Br; I; C₁, to C₆ straight chain or branched chain alkyl; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a phosphate thereof; PO₂(OCH₃)H or a phosphonate thereof; NO₂; NH₂; NHCH(O); NRCH(O); NHC(O)R; NRC(O)R₁; C(O)NRR₁; C(NH)NRR₁; C(NH)NROH; C(NH)NRNO2; or C(NR)NR₁C(NR₂)NR₃R₄;

wherein adjacent substituents U, V and W or X, Y and Z may form a saturated or unsaturated 5-membered or 6-membered carbocyclic or heterocyclic ring;

wherein each of R, R₁, R₂, R₃ and R₄ if present is independently:

H; OH; O-Rx; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

wherein Rx, if present, is alkyl, cycloalkyl, alkylcycloalkyl, acyl, ester or thioester;

and wherein A is CH or N.

The subject invention also provides a method of treating a disease condition in a subject which comprises administering to the subject a composition comprising a pharmaceutically acceptable carrier and an amount of a compound or of a pharmaceutically acceptable salt of the compound effective to treat the disease condition, wherein the compound has the structure:

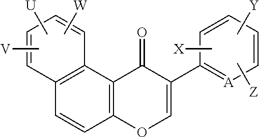

wherein:

each of U, V, W, X, Y, and Z is independently:

H; OH; F; Cl; Br; I; C₁ to C₆ straight chain or branched chain alkyl; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a or a phosphate thereof; PO₂(OCH₃)H or a phosphonate thereof; NO₂; NH₂; NHCH(O); NRCH(O); NHC(O)R; NRC(O)R₁; C(O)NRR₁; C(NH)NRR₁; C(NH)NROH; C(NH)NRNO2; or C(NR)NR₁C(NR₂)NR₃R₄;

wherein adjacent substituents U, V and W or X, Y and Z may form a saturated or unsaturated 5-membered or 6-membered carbocyclic or heterocyclic ring;

wherein each of R, R₁, R₂, R₃ and R₄ if present is independently:

H; OH; O-Rx; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

wherein Rx, if present, is alkyl, cycloalkyl, alkylcycloalkyl, acyl, ester or thioester;

and wherein A is CH or N;

and wherein the disease condition Is pain, inflammation, neurodegenerative diseases, neuropathic pain, trigeminal neuralgia, postherpetic neuralgia, diabetic neuropathy, cancer pain, phantom limb pain, complex regional pain syndrome, and fibromyalgia; rheumatoid arthritis, ankolysing spondylitis, ulcerative colitis, tendonitis, psoriasis, Hidradenitis Suppurativa (sometimes referred to as Acne Inversa),

Faber's Disease, Crohn's Disease, rhinitis, skin allergies, asthma, autoimmune diseases with inflammatory components such as multiple sclerosis and other demyelinating disorders; Alzheimer's Disease, traumatic brain Injury, conditions and diseases characterized by abnormal lipid metabolism and secretion, metabolic disorders, appetite regulation, or obesity.

The subject invention further provides a method of treating excess fat in a subject which comprises administering to an area of excess fat a composition comprising a pharmaceutically acceptable carrier and an amount of a compound or of a pharmaceutically acceptable salt of the compound effective to treat the excess fat, wherein the compound has the structure:

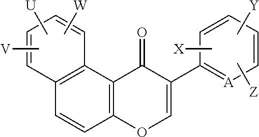

wherein:

each of U, V, W, X, V, and Z is independently:

H; OH; F; Cl; Br; I; C₁ to C₆ straight chain or branched chain alkyl; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a phosphate thereof; PO₂(OCH₃)H or a phosphonate thereof; NO₂; NH₂; NHCH(O); NRCH(O); NHC(O)R; NRC(O)R₁; C(O)NRR₁; C(NH)NRR₁; C(NH)NROH; C(NH)NRNO2; or C(NR)NR₁C(NR₂)NR₃R₄;

wherein adjacent substituents U, V and W or X, Y and Z may form a saturated or unsaturated 5-membered or 6-membered carbocyclic or heterocyclic ring;

wherein each of R, R₁, R₂, R₃ and R₄ if present is independently:

H; OH; O-Rx; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

wherein Rx, if present, is alkyl, cycloalkyl, alkylcycloalkyl, acyl, ester or thioester;

and wherein A is CH or N.

The subject invention still further provides a pharmaceutical composition comprising a pharmaceutically acceptable carrier and a compound or of a pharmaceutically acceptable salt of the compound, wherein the compound has the structure:

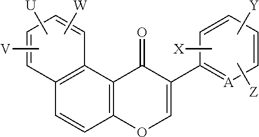

wherein:

each of U, V, W, X, Y, and Z is independently:

H; OH; F; Cl; Br; I; C₁ to C6 straight chain or branched chain alkyl; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a phosphate thereofor a phosphate thereof; PO₂(OCH₃)H or a phosphonate thereof; NO₂; NH₂; NHCH(O); NRCH(O); NHC(O)R; NRC(O)R₁; C(O)NRR₁; C(NH)NRR₁; C(NH)NROH; C(NH)NRNO2; or C(NR)NR₁C(NR₂)NR₃R₄;

wherein adjacent substituents U, V and W or X, Y and Z may form a saturated or unsaturated 5-membered or 6-membered carbocyclic or heterocyclic ring;

wherein each of R, R₁, R₂, R₃ and R₄ if present is independently:

H; OH; O-Rx; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

wherein Rx, if present, is alkyl, cycloalkyl, alkylcycloalkyl, acyl, ester or thioester;

and wherein A is CH or N.

The subject invention yet further provides a compound having the structure I, or a pharmaceutically acceptable salt thereof,

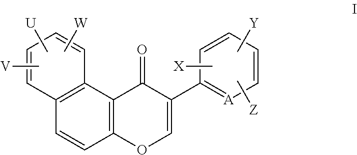

wherein:

each of U, V, W, X, Y, and Z is independently:

H; OH; F; Cl; Br; I; C₁ to C₆ straight chain or branched chain alkyl; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a phosphate thereofor a phosphate thereof; PO₂(OCH₃)H or a phosphonate thereof; NO₂; NH₂; NHCH(O); NRCH(O); NHC(O)R; NRC(O)R₁; C(O)NRR₁; C(NH)NRR₁; C(NH)NROH; C(NH)NRNO2; or C(NR)NR₁C(NR₂)NR₃R₄;

wherein adjacent substituents U, V and W or X, Y and Z may form a saturated or unsaturated 5-membered or 6-membered carbocyclic or heterocyclic ring;

wherein each of R, R₁, R₂, R₃ and R₄ if present is independently:

H; OH; O-Rx; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

wherein Rx, if present, is alkyl, cycloalkyl, alkylcycloalkyl, acyl, ester or thioester;

and wherein A is CH or N.

### BRIEF DESCRIPTION OF THE FIGURES

FIG. 1 shows the results in vitro (HepG2 cells) measuring the fold induction of CYP1A1 vs. concentration in the EROD assay as described in Example 8.

FIG. 2 shows histopathology of murine ear slices stained according to methodology described in Example 12 after exposure to either a vehicle control (upper slide) or compound B (lower slide). The sebaceous glands as well as their contents are clearly visible and can be measured

FIG. 3 shows the ratio of differentiated sebocytes vs. undifferentiated sebocytes per sebaceous gland for a range of compounds, including compound B vs. a vehicle control. FIG. 3 is derived from murine ear slice data, such as those shown in FIG. 2.

FIG. 4 shows the number of sebaceous glands per mm² for a range of compounds, including compound B vs. a vehicle control. FIG. 4 is derived from murine ear slice data, such as those shown in FIGS. 2.

### DETAILED DESCRIPTION OF THE INVENTION

The subject invention provides a method of treating a skin condition associated with abnormal sebum secretion or abnormal sebaceous gland function in a subject which comprises topically and periodically applying to an area of subject's skin affected by the skin condition a composition comprising a pharmaceutically acceptable carrier and an amount of a compound or of a pharmaceutically acceptable salt of the compound effective to treat the skin condition, wherein the compound has structure

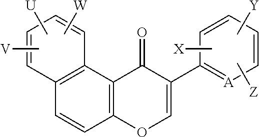

wherein:

each of U, V, W, X, Y, and Z is independently:

H; OH; F; Cl; Br; I; C₁ to CS straight chain or branched chain alkyl; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₃)—R; SO₃H or an ester thereof; CO₂H or an ester thereof; PO₃H₂ or a phosphate thereofor a phosphate thereof; PO₂(OCH₃)H ora phosphonate thereof; NO₂; NH₂; NHCH(O); NRCH(O); NHC(O)R; NRC(O)R₁; C(O)NRR₁; C(NH)NRR₁; C(NH)NROH; C(NH)NRNO2; or C(NR)NR₁C(NR₂)NR₃R₄;

wherein adjacent substituents U, V and W or X, Y and Z may form a saturated or unsaturated 5-membered or 6-membered carbocyclic or heterocyclic ring;

wherein each of R, R₁, R₂, R₃ and R₄ if present is independently:

H; OH; O-Rx; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

wherein Rx, if present, is alkyl, cycloalkyl, alkylcycloalkyl, acyl, ester or thioester;

and wherein A is CH or N.

In some embodiments, adjacent substituents U, V and W and X, Y and Z may form a saturated or unsaturated 5-membered or 6-membered carbocyclic or heterocyclic ring.

In further embodiments, each of U, V, W, X, Y, and Z is independently: H; OH; F; Cl; Br; I; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a phosphate thereof; PO₂(OCH₃)H or a phosphonate thereof; NH₂; NHCH(O); NRCH(O); NHC(O)R; NRC(O)R₁; C(O)NRR₁; C(NH)NRR₁; C(NH)NROH; or C(NR)NR₁C(NR₂)NR₃R₄;

each of R, R₁, R₂, R₃ and R₄ if present is independently: H, OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

and wherein A is CH or N.

In further embodiments, each of U, V, W, X, Y, and Z is independently:

H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;

each of R and R₁ if present is independently: H; OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

and wherein A is CH or N.

In other embodiments, each of U, V, W, X, Y, and Z is independently:

H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;

each of R and R₁ if present is independently: H; OH; optionally substituted alkyl; cycloalkyl; alkylcycloalkyl; heterocycloalkyl; alkylheterocycloalkyl; optionally substituted alkenyl; or optionally substituted alkynyl;

and wherein A is CH or N.

In further embodiments, at least one of U, V and W is H and at least one of X, Y and Z is H.

In other embodiments, at least two of U, V and W is H or at least two of X,)(and Z is H.

In yet other embodiments, each of U, V and W is H or each of X, Y and Z is H.

In additional embodiments, one of U, V and W is H and each of X, Y and Z is H; one of X, Y and Z is H and each of U, V and W is H; two of U, V and W is H and each of X, Y and Z is H; or two of X, Y and Z is H and each of U, V and W is H.

In yet further embodiments, at least one of U, V W, X, Y and Z is other than H.

In certain embodiments, the compound is one of the following:

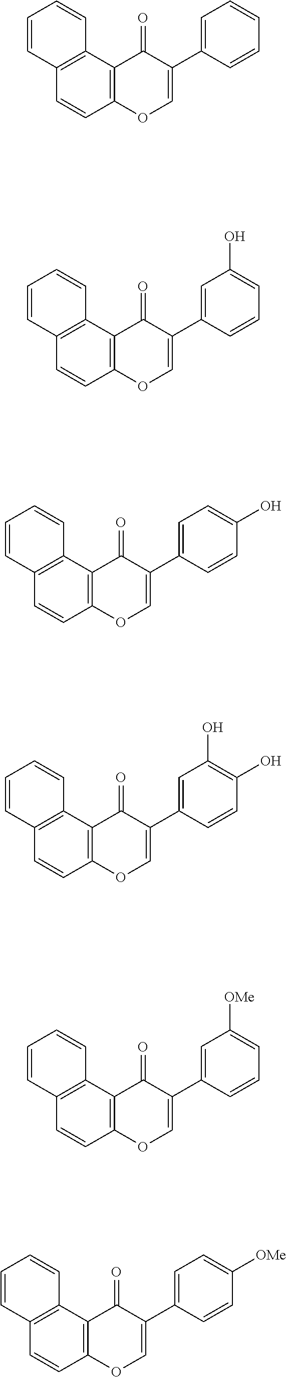 [SMILES: O=c1c(-c2ccccc2)coc2ccc3ccccc3c12 ; O=c1c(-c2cccc(O)c2)coc2ccc3ccccc3c12 ; O=c1c(-c2ccc(O)cc2)coc2ccc3ccccc3c12 ; O=c1c(-c2ccc(O)c(O)c2)coc2ccc3ccccc3c12 ; COc1cccc(-c2coc3ccc4ccccc4c3c2=O)c1 ; COc1ccc(-c2coc3ccc4ccccc4c3c2=O)cc1]

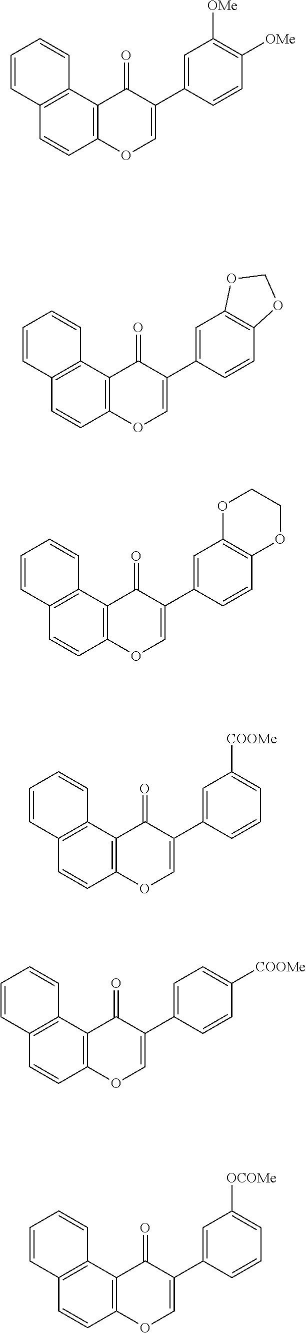

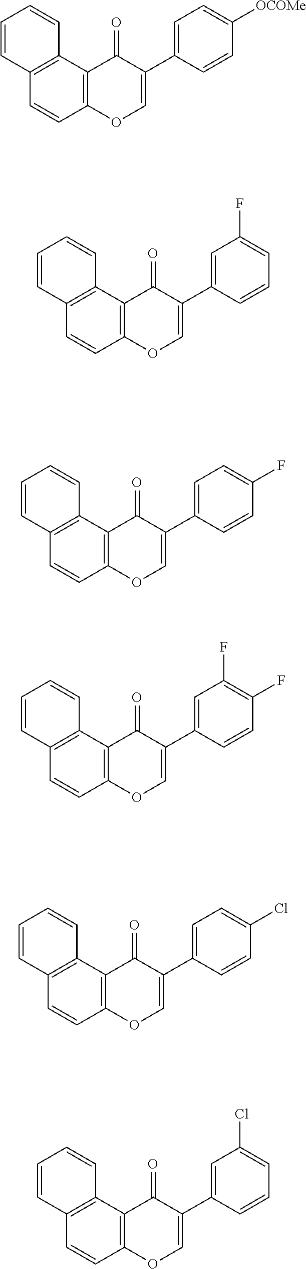

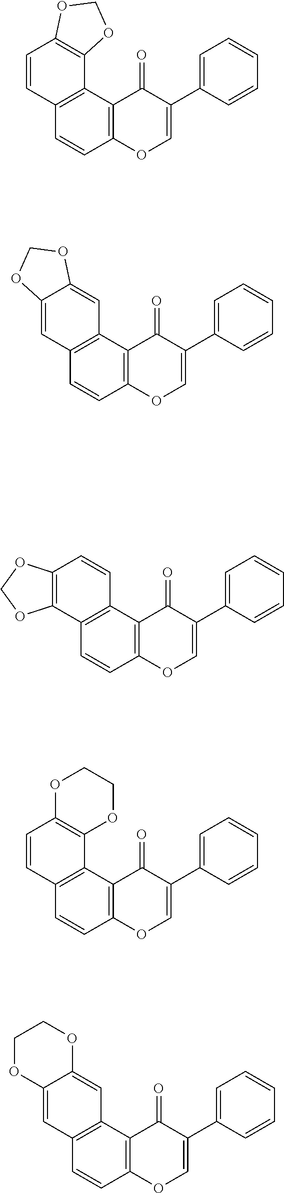 [SMILES: O=c1c(-c2ccccc2)coc2ccc3ccc4c(c3c12)OCO4 ; O=c1c(-c2ccccc2)coc2ccc3cc4c(cc3c12)OCO4 ; O=c1c(-c2ccccc2)coc2ccc3c4c(ccc3c12)OCO4 ; O=c1c(-c2ccccc2)coc2ccc3ccc4c(c3c12)OCCO4 ; O=c1c(-c2ccccc2)coc2ccc3cc4c(cc3c12)OCCO4]

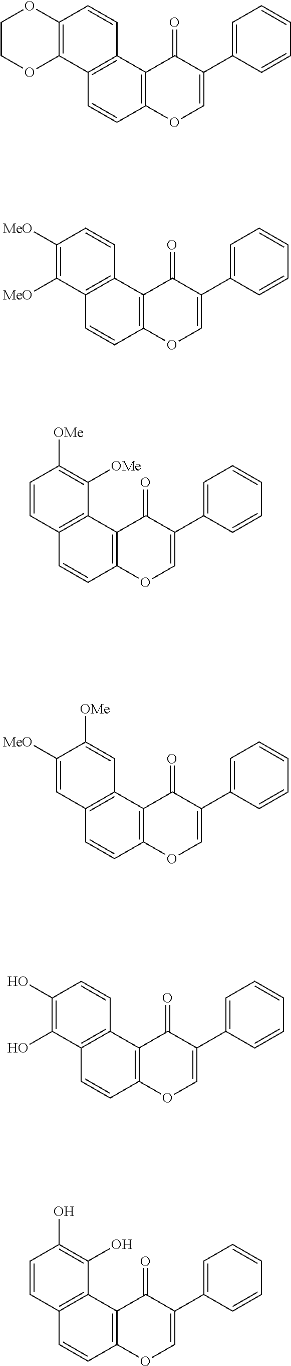 [SMILES: O=c1c(-c2ccccc2)coc2ccc3c4c(ccc3c12)OCCO4 ; COc1ccc2c(ccc3occ(-c4ccccc4)c(=O)c32)c1OC ; COc1ccc2ccc3occ(-c4ccccc4)c(=O)c3c2c1OC ; COc1cc2ccc3occ(-c4ccccc4)c(=O)c3c2cc1OC ; O=c1c(-c2ccccc2)coc2ccc3c(O)c(O)ccc3c12 ; O=c1c(-c2ccccc2)coc2ccc3ccc(O)c(O)c3c12]

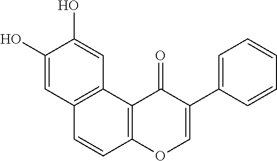 [SMILES: O=c1c(-c2ccccc2)coc2ccc3cc(O)c(O)cc3c12]

or a pharmaceutically acceptable salt, ester or prodrug form thereof.

In certain other embodiments, the compound is one of the following:

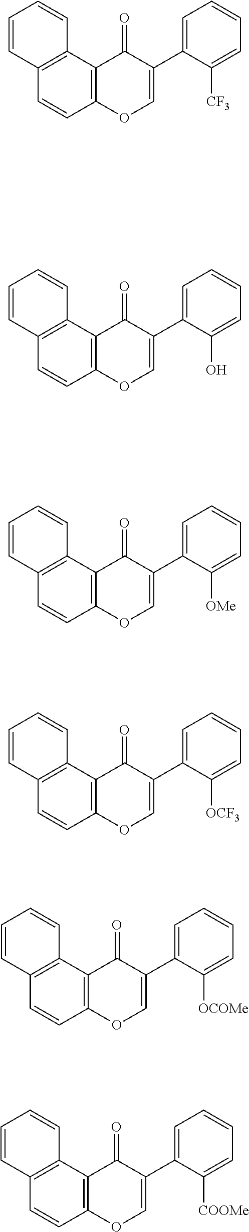

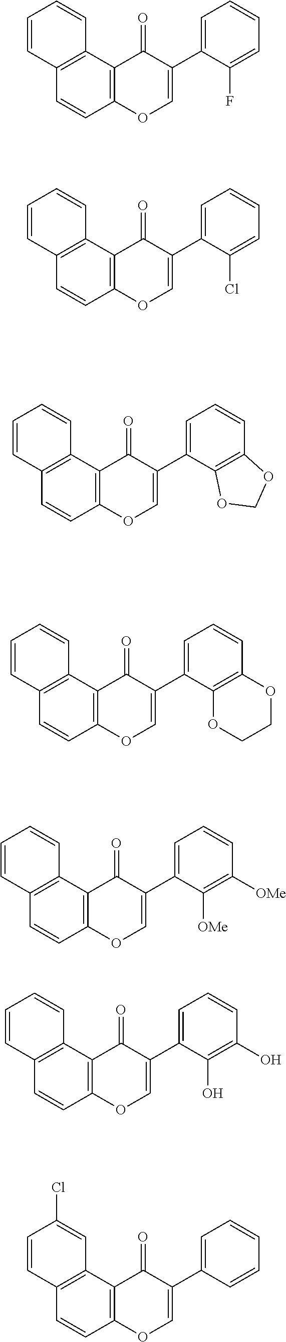

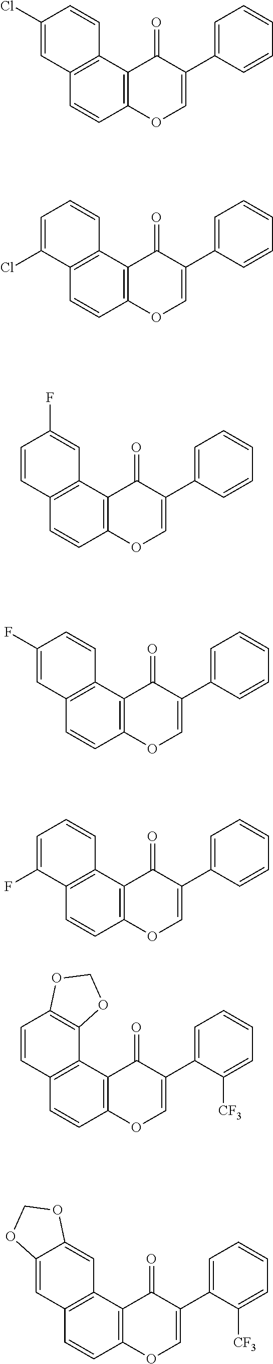

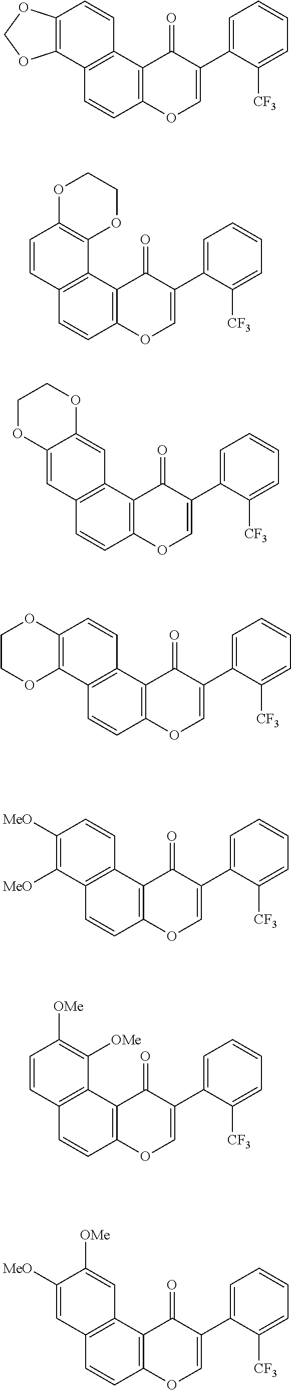

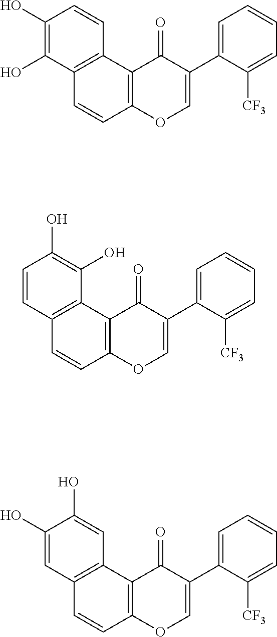 [SMILES: O=c1c(-c2ccccc2C(F)(F)F)coc2ccc3c(O)c(O)ccc3c12 ; O=c1c(-c2ccccc2C(F)(F)F)coc2ccc3ccc(O)c(O)c3c12 ; O=c1c(-c2ccccc2C(F)(F)F)coc2ccc3cc(O)c(O)cc3c12]

or a pharmaceutically acceptable salt, ester or prodrug form thereof.

In certain other embodiments, the compound is one of the following:

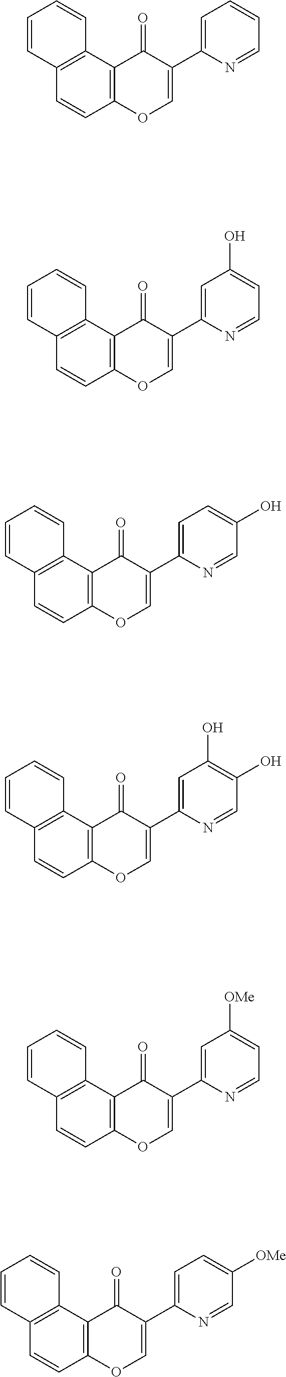 [SMILES: O=c1c(-c2ccccn2)coc2ccc3ccccc3c12 ; O=c1c(-c2cc(O)ccn2)coc2ccc3ccccc3c12 ; O=c1c(-c2ccc(O)cn2)coc2ccc3ccccc3c12 ; O=c1c(-c2cc(O)c(O)cn2)coc2ccc3ccccc3c12 ; COc1ccnc(-c2coc3ccc4ccccc4c3c2=O)c1 ; COc1ccc(-c2coc3ccc4ccccc4c3c2=O)nc1]

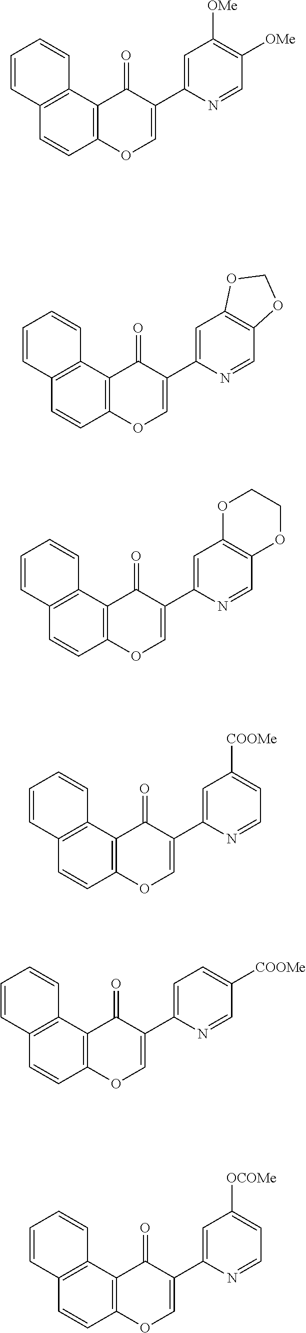

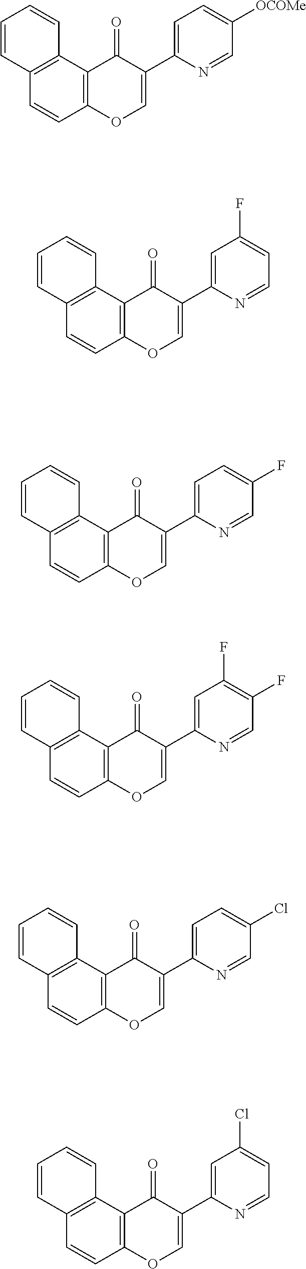

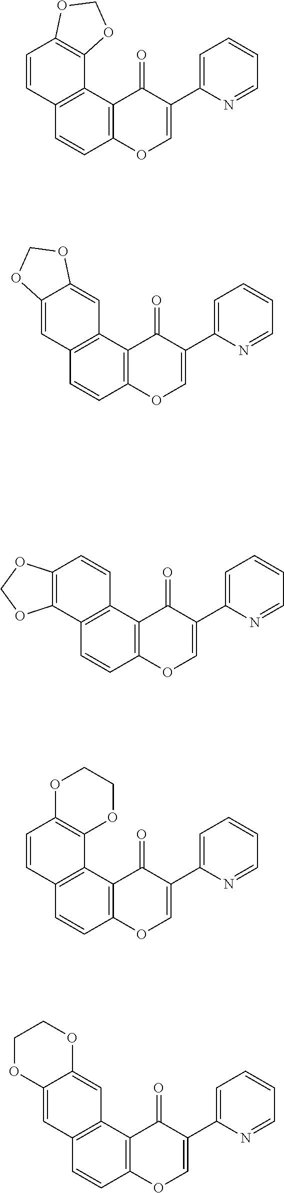 [SMILES: O=c1c(-c2ccccn2)coc2ccc3ccc4c(c3c12)OCO4 ; O=c1c(-c2ccccn2)coc2ccc3cc4c(cc3c12)OCO4 ; O=c1c(-c2ccccn2)coc2ccc3c4c(ccc3c12)OCO4 ; O=c1c(-c2ccccn2)coc2ccc3ccc4c(c3c12)OCCO4 ; O=c1c(-c2ccccn2)coc2ccc3cc4c(cc3c12)OCCO4]

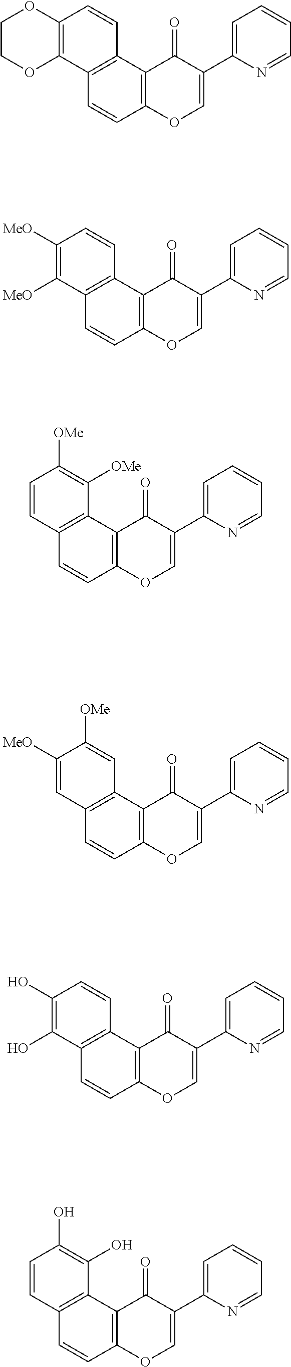 [SMILES: O=c1c(-c2ccccn2)coc2ccc3c4c(ccc3c12)OCCO4 ; COc1ccc2c(ccc3occ(-c4ccccn4)c(=O)c32)c1OC ; COc1ccc2ccc3occ(-c4ccccn4)c(=O)c3c2c1OC ; COc1cc2ccc3occ(-c4ccccn4)c(=O)c3c2cc1OC ; O=c1c(-c2ccccn2)coc2ccc3c(O)c(O)ccc3c12 ; O=c1c(-c2ccccn2)coc2ccc3ccc(O)c(O)c3c12]

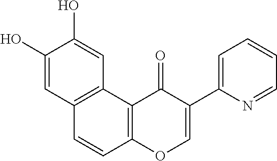 [SMILES: O=c1c(-c2ccccn2)coc2ccc3cc(O)c(O)cc3c12]

or a pharmaceutically acceptable salt, ester or prodrug form thereof.

In certain other embodiments, the compound is one of the following:

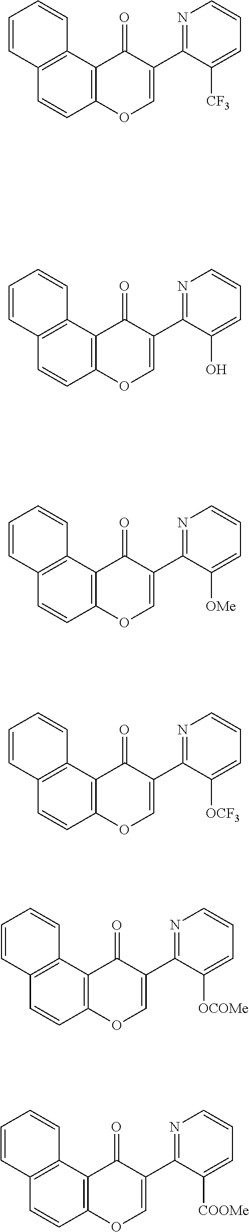

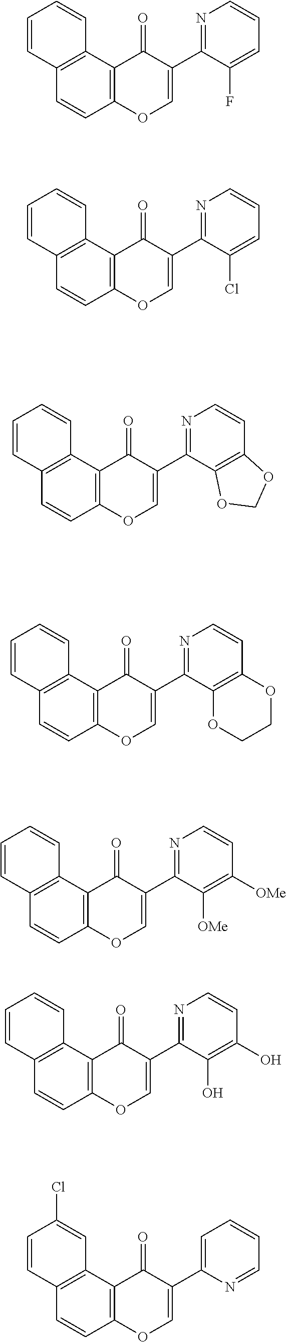

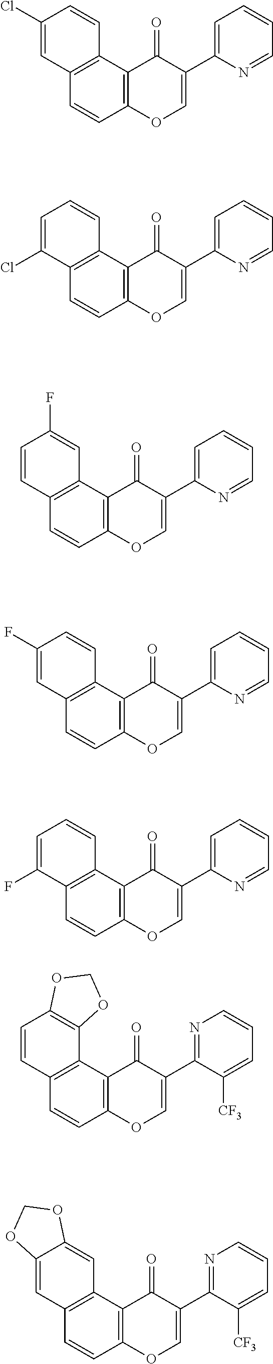

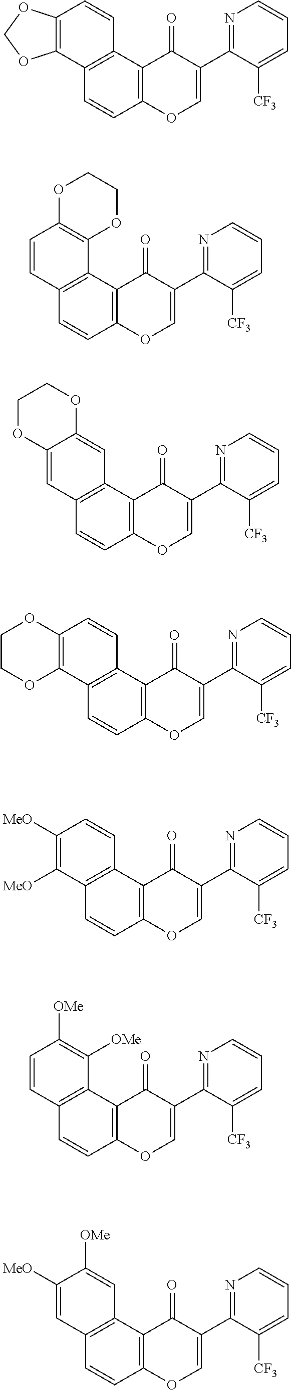

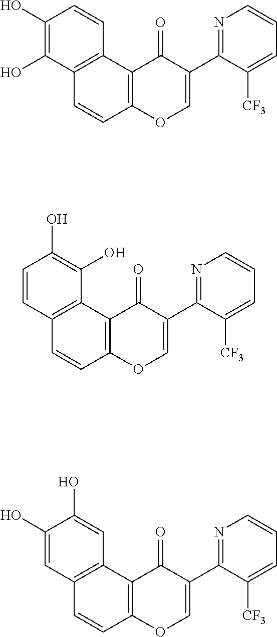 [SMILES: O=c1c(-c2ncccc2C(F)(F)F)coc2ccc3c(O)c(O)ccc3c12 ; O=c1c(-c2ncccc2C(F)(F)F)coc2ccc3ccc(O)c(O)c3c12 ; O=c1c(-c2ncccc2C(F)(F)F)coc2ccc3cc(O)c(O)cc3c12]

or a pharmaceutically acceptable salt, ester or prodrug form thereof.

In some embodiments, an asymmetric center is present in the compound, and the compound is a racemic mixture, a diastereoisomeric mixture, a single enantiomer, an enantiomeric diastereomer, a meso compound, a pure epimer, or a mixture of epimers thereof.

In other embodiments, one or more double bonds present in the compound are cis or trans, E or Z, a cis/trans mixture, an E/Z mixture, a combination of E and Z geometries, a combination of E and Z geometric mixtures or other geometric isomers thereof.

In yet other embodiments, the compound has a lipophilicity as measured by LogP greater than 3.

In further embodiments, the pharmaceutically acceptable carrier is suitable for topical use.

In further embodiments, the pharmaceutically acceptable carrier is suitable for intralesional use.

In some embodiments, the compound has at least one of the following properties:
a) an ability to activate the AhR receptor,
b) an ability to modulate a gene regulated by AhR,
c) an ability to down regulate the expression of genes involved in the synthesis of lipids in sebum,
d) an ability to modulate one or several enzymes involved in lipid metabolism,
e) a short half-life in the human organism of between 0 hours and 96 h, and
f) a measurable positive effect on a recognized criterion of sebaceous hyperactivity.

In another embodiment, the compound has an ability to down regulate the expression of anti-Inflammatory genes such as ALOX-15. In a separate embodiment, the compound has an ability to up regulate the expression of anti-inflammatory genes such as ALOX-15.

In some embodiments, the skin condition is oily skin, oily hair, shiny or greasy-looking skin, hyperseborrhea, acne, se borrheic dermatitis, rosacea, sebaceous hyperplasia or sebaceous carcinoma.

In alternative embodiments, the skin condition is acne, seborrheic dermatitis, rosacea, hyperseborrhea, sebaceous hyperplasia or sebaceous carcinoma.

In some embodiments, the compound is present in the composition at a concentration of between about 0.005% and about 5% by weight.

The present invention also provides a method of treating a disease condition in a subject which comprises administering to the subject a composition comprising a pharmaceutically acceptable carrier and an amount of a compound or of a pharmaceutically acceptable salt of the compound effective to treat the disease condition, wherein the compound has the structure:

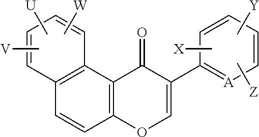

wherein:

each of U, V, W, X, V. and Z is independently:

H; OH; F; Cl; Br; I; C₁ to C₆ straight chain or branched chain alkyl; CH₂F; CHF₂; CF₃; O-alkyl; O-cycloalkyl; O-alkylcycloalkyl; OCH₂F; OCH F₂; OCF₃; O—(CO)—R; O—(CNH)—R; O—(CNRO-R; SO₃H or an ester thereof; CO₂H or an ester thereof; PO₃H₂ or a phosphate thereof; PO₂(OCH₃)H or a phosphonate thereof; NO₂; NH₂; NHCH(O); NRCH(O); NHC(O)R; NRC(O)R₁; C(O)NRR₁; C(NH)NRR₁; C(NH)NROH; C(NH)NRNO2; or C(NR)NR₁C(NR₂)NR₃R₄;

wherein adjacent substituents U, V and W or X, Y and Z may form a saturated or unsaturated 5-membered or 6-membered carbocyclic or heterocyclic ring;

wherein each of R, R₁, R₂, R₃ and R₄ if present is independently: H; OH; O-Rx; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

wherein Rx, if present, is alkyl, cycloalkyl, alkylcycloalkyl, acyl, ester or thioester;

and wherein A is CH or N;

and wherein the disease condition is pain, inflammation, neurodegenerative diseases, neuropathic pain, trigeminal neuralgia, postherpetic neuralgia, diabetic neuropathy, cancer pain, phantom limb pain, complex regional pain syndrome, and fibromyalgia; rheumatoid arthritis, ankolysing spondylitis, ulcerative colitis, tendonitis, psoriasis, Hidradenitis Suppurativa (sometimes referred to as Acne Inverse), Faber's Disease, Crohn's Disease, rhinitis, skin allergies, asthma, autoimmune diseases with inflammatory components such as multiple sclerosis and other demyelinating disorders; Alzheimer's Disease, traumatic brain injury, conditions and diseases characterized by abnormal lipid metabolism and secretion, metabolic disorders, appetite regulation, or obesity.

In certain embodiments, the disease condition is inflammation, psoriasis or obesity.

In a specific embodiment, the disease condition is psoriasis.

The present invention further provides a method of treating excess fat in a subject which comprises administering to an area of excess fat a composition comprising a pharmaceutically acceptable carrier and an amount of a compound or of a pharmaceutically acceptable salt of the compound effective to treat the excess fat, wherein the compound has the structure:

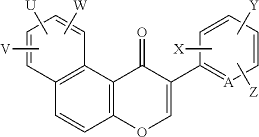

wherein:

each of U, V, W, X, Y, and Z is independently:

H; OH; F; Cl; Br; I; C₁ to C₆ straight chain or branched chain alkyl; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a phosphate thereof; PO₂(OCH₃)H or a phosphonate thereof; NO₂; NH₂; NHCH(O); NRCH(O); NHC(O)R; NRC(O)R₁; C(O)NRR₁; C(NH)NRR₁; C(NH)NROH; C(NH)NRNO2; or C(NR)NR₁C(NR₂)NR₃R4;

wherein adjacent substituents U, V and W or X, Y and Z may form a saturated or unsaturated 5-membered or 6-membered carbocyclic or heterocyclic ring;

wherein each of R, R₁ₛ R₂, R₃ and R₄ if present is independently;

H; OH; O-Rx; optionally substituted alkyl; cycloalkyl; alkylcycloalkyl; heterocycloaikyi; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

wherein Rx, if present, is alkyl, cycloalkyl, alkylcycloalkyl, acyl, ester or thioester;

and wherein A is CH or N.

In certain embodiments, the excess fat is a lipoma, a liposarcoma or an excess of submental fat. In certain embodiments, the excess fat is abnormal adipose cells or tissue. In other embodiments, the excess fat is an excess of eyelid fat (steatoblepharon, including either or both upper and lower steatoblepharon), otherwise known as eye bags. In yet other embodiments, the excess fat is surrounding the eye and is associated with Grave's opthalmopathy.

In some embodiments, adjacent substituents U, V and W and X, Y and Z may form a saturated or unsaturated 5-membered or 6-membered carbocyclic or heterocyclic ring.

In further embodiments, each of U, V, W, X, Y, and Z is independently: H; OH; F; Cl; Br; I; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a phosphate thereof; PO₂(OCH₃)H or a phosphonate thereof; NH₂; NHCH(O); NRCH(O); NHC(O)R; NRC(O)R₃; C(O)NRR₁; C(NH)NRR₁; C(NH)NROH; or C(NR)NR₁C(NR₂)NR₃R₄;

each of R, R₂, R₂, R₃ and R₄ if present is independently: H, OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

and wherein A is CH or N.

In further embodiments, each of U, V, W, X, V. and Z is independently:

H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;

each of R and R₁ if present is independently: H; OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

and wherein A is CH or N.

In other embodiments, each of U, V, W, X, Y, and Z is independently:

H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;

each of R and R₁ if present is independently: H; OH; optionally substituted alkyl; cycloalkyl; alkylcycloalkyl; heterocycloalkyl; alkylheterocycloalkyl; optionally substituted alkenyl; or optionally substituted alkynyl;

and wherein A is CH or N.

In further embodiments, at least one of U, V and W is H and at least one of X, Y and Z is H.

In other embodiments, at least two of U, V and W is H or at least two of X, Y and Z is H.

In yet other embodiments, each of U, V and W is H or each of X, Y and Z is H.

In additional embodiments, one of U, V and W is H and each of X, Y and Z is H; one of X, Y and Z is H and each of U, V and W is H; two of U, V and W is H and each of X, Y and Z is H; or two of X, Y and Z is H and each of U, V and W is H.

In yet further embodiments, at least one of U, V W, X, Y and Z is other than H.

In certain embodiments, the compound is one of the following:

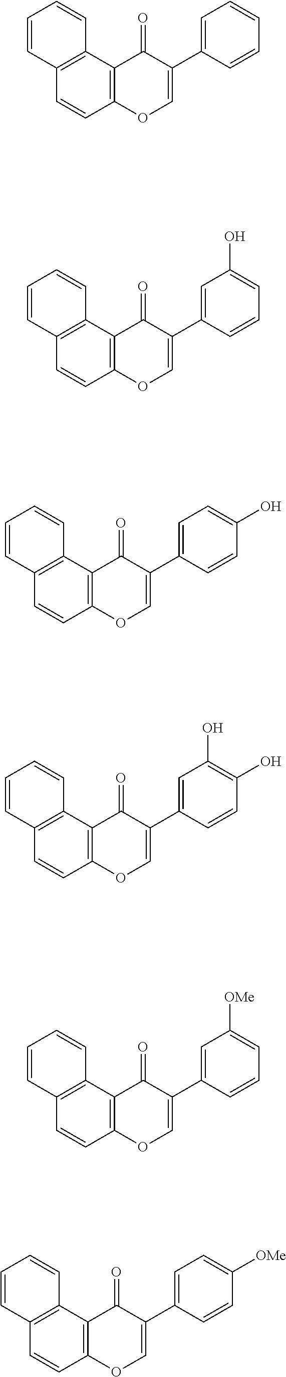 [SMILES: O=c1c(-c2ccccc2)coc2ccc3ccccc3c12 ; O=c1c(-c2cccc(O)c2)coc2ccc3ccccc3c12 ; O=c1c(-c2ccc(O)cc2)coc2ccc3ccccc3c12 ; O=c1c(-c2ccc(O)c(O)c2)coc2ccc3ccccc3c12 ; COc1cccc(-c2coc3ccc4ccccc4c3c2=O)c1 ; COc1ccc(-c2coc3ccc4ccccc4c3c2=O)cc1]

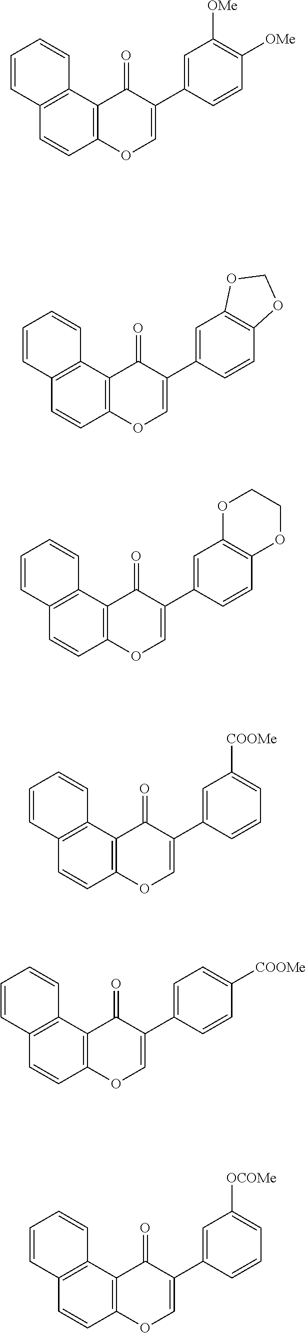

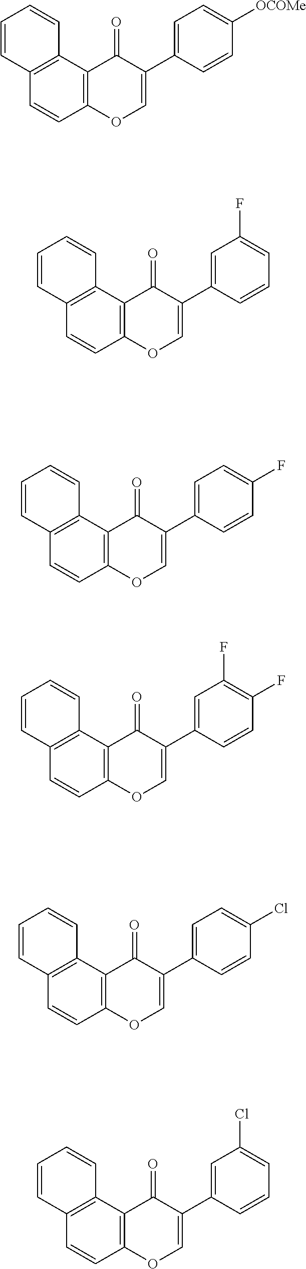

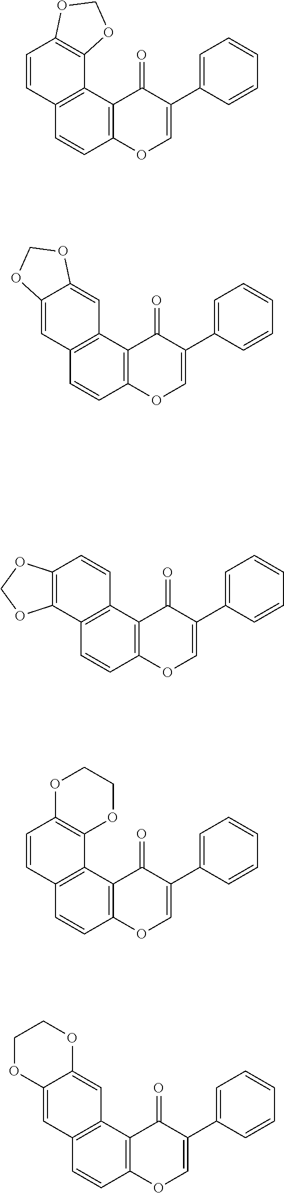 [SMILES: O=c1c(-c2ccccc2)coc2ccc3ccc4c(c3c12)OCO4 ; O=c1c(-c2ccccc2)coc2ccc3cc4c(cc3c12)OCO4 ; O=c1c(-c2ccccc2)coc2ccc3c4c(ccc3c12)OCO4 ; O=c1c(-c2ccccc2)coc2ccc3ccc4c(c3c12)OCCO4 ; O=c1c(-c2ccccc2)coc2ccc3cc4c(cc3c12)OCCO4]

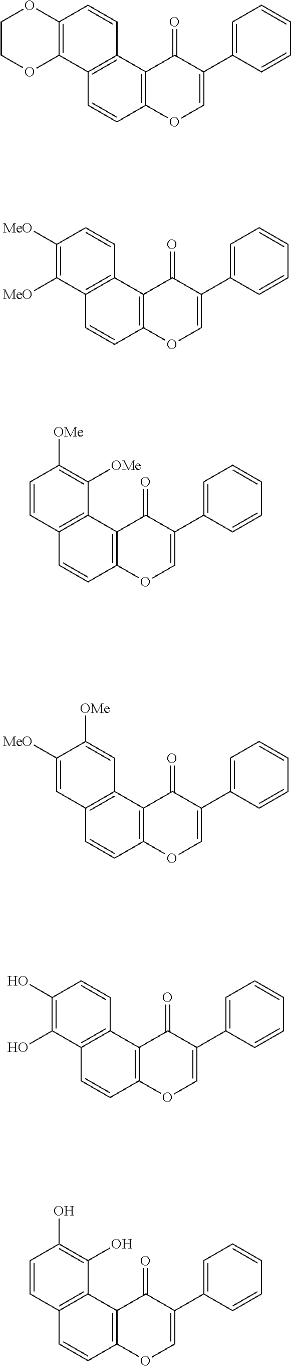 [SMILES: O=c1c(-c2ccccc2)coc2ccc3c4c(ccc3c12)OCCO4 ; COc1ccc2c(ccc3occ(-c4ccccc4)c(=O)c32)c1OC ; COc1ccc2ccc3occ(-c4ccccc4)c(=O)c3c2c1OC ; COc1cc2ccc3occ(-c4ccccc4)c(=O)c3c2cc1OC ; O=c1c(-c2ccccc2)coc2ccc3c(O)c(O)ccc3c12 ; O=c1c(-c2ccccc2)coc2ccc3ccc(O)c(O)c3c12]

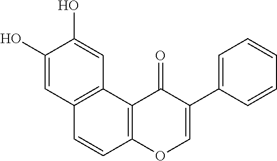 [SMILES: O=c1c(-c2ccccc2)coc2ccc3cc(O)c(O)cc3c12]

or a pharmaceutically acceptable salt, ester or prodrug form thereof.

In certain other embodiments, the compound is one of the following:

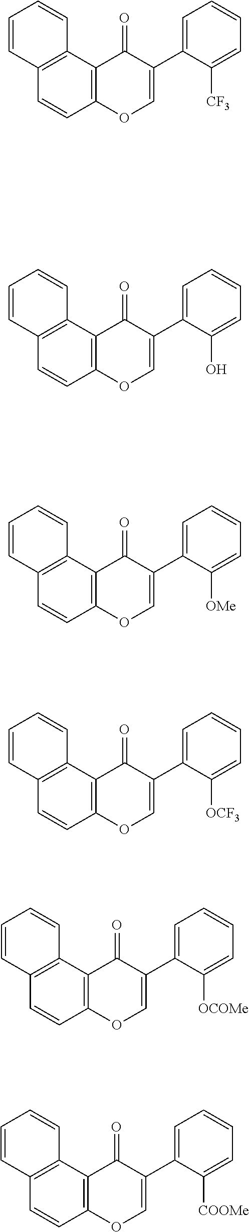

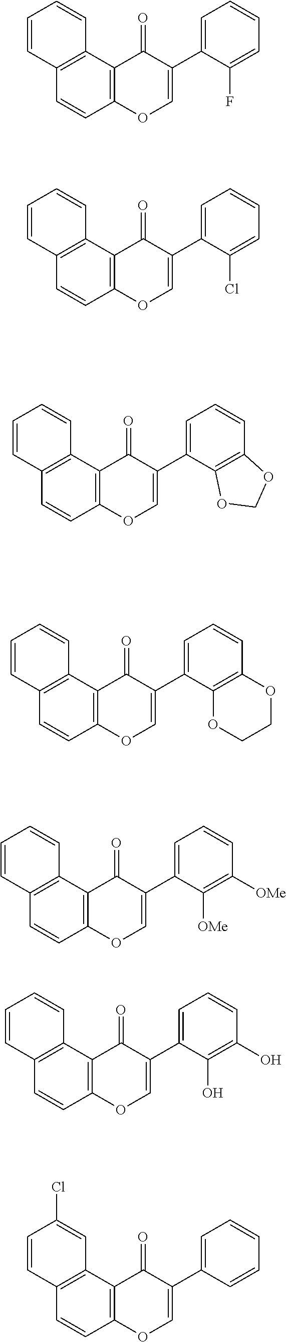

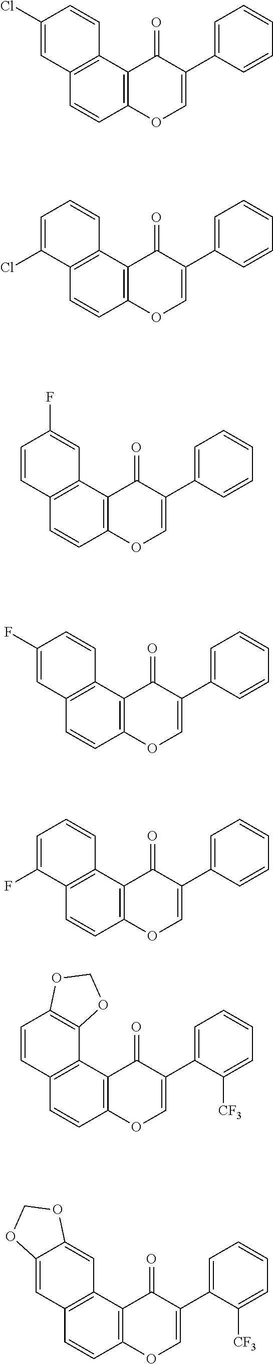

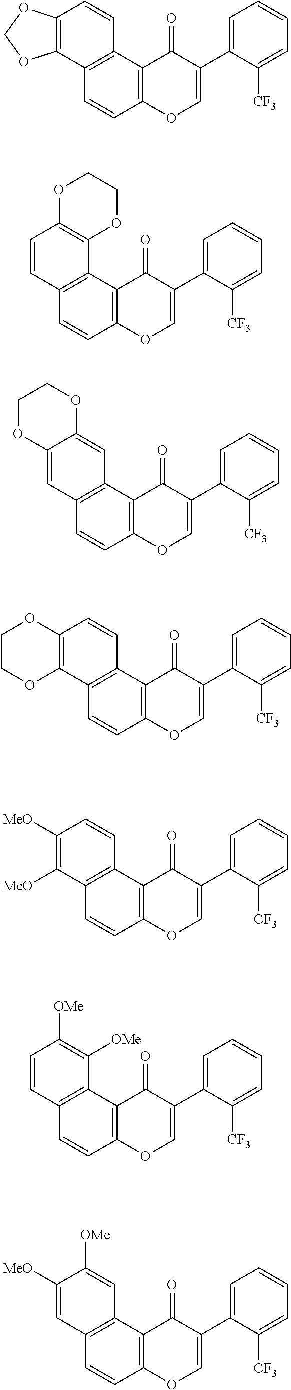

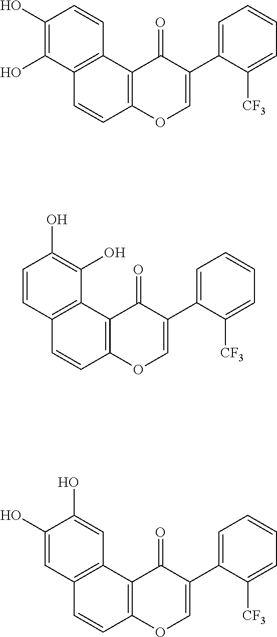 [SMILES: O=c1c(-c2ccccc2C(F)(F)F)coc2ccc3c(O)c(O)ccc3c12 ; O=c1c(-c2ccccc2C(F)(F)F)coc2ccc3ccc(O)c(O)c3c12 ; O=c1c(-c2ccccc2C(F)(F)F)coc2ccc3cc(O)c(O)cc3c12]

or a pharmaceutically acceptable salt, ester or prodrug form thereof.

In certain other embodiments, the compound is one of the following:

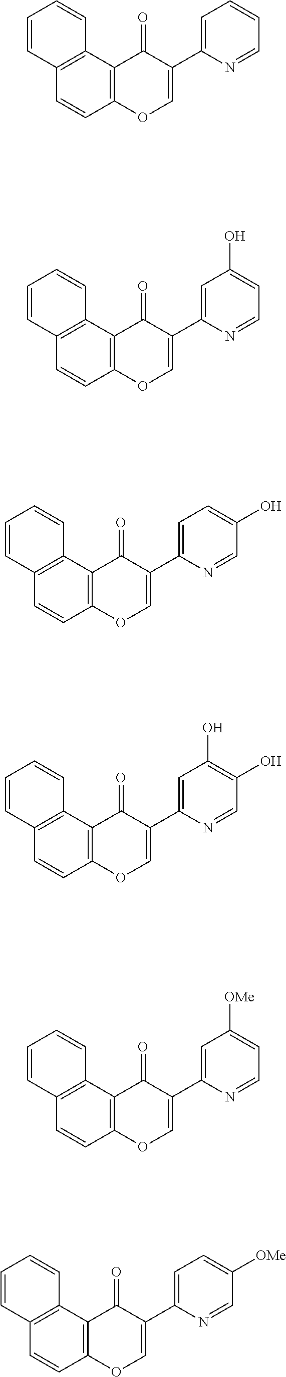 [SMILES: O=c1c(-c2ccccn2)coc2ccc3ccccc3c12 ; O=c1c(-c2cc(O)ccn2)coc2ccc3ccccc3c12 ; O=c1c(-c2ccc(O)cn2)coc2ccc3ccccc3c12 ; O=c1c(-c2cc(O)c(O)cn2)coc2ccc3ccccc3c12 ; COc1ccnc(-c2coc3ccc4ccccc4c3c2=O)c1 ; COc1ccc(-c2coc3ccc4ccccc4c3c2=O)nc1]

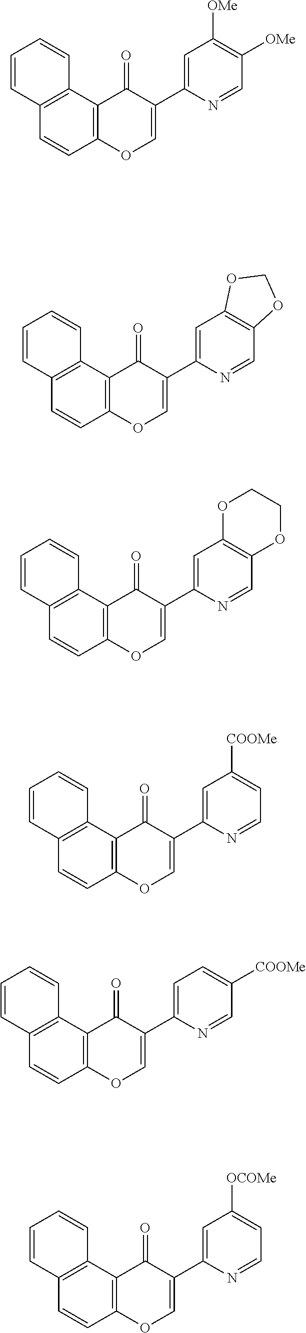

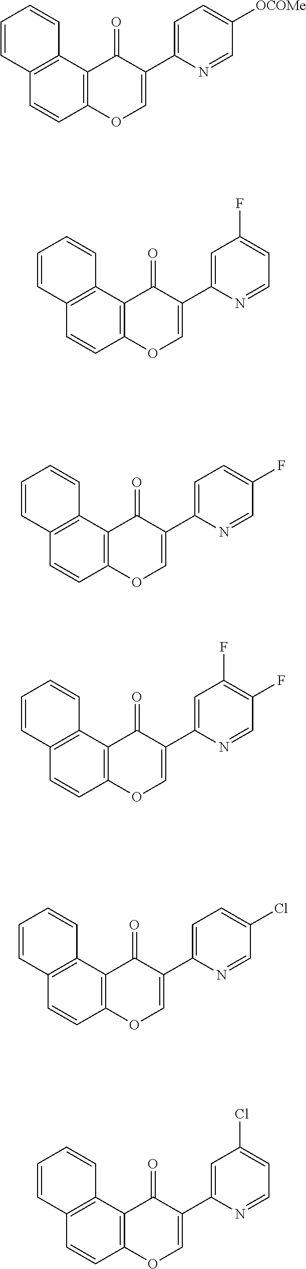

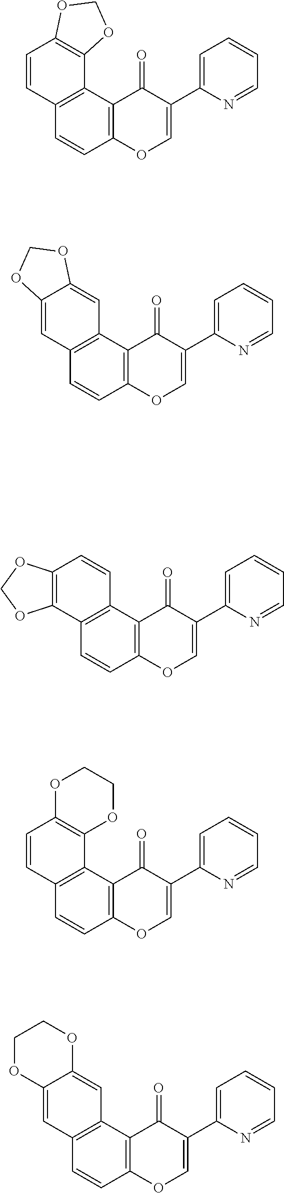 [SMILES: O=c1c(-c2ccccn2)coc2ccc3ccc4c(c3c12)OCO4 ; O=c1c(-c2ccccn2)coc2ccc3cc4c(cc3c12)OCO4 ; O=c1c(-c2ccccn2)coc2ccc3c4c(ccc3c12)OCO4 ; O=c1c(-c2ccccn2)coc2ccc3ccc4c(c3c12)OCCO4 ; O=c1c(-c2ccccn2)coc2ccc3cc4c(cc3c12)OCCO4]

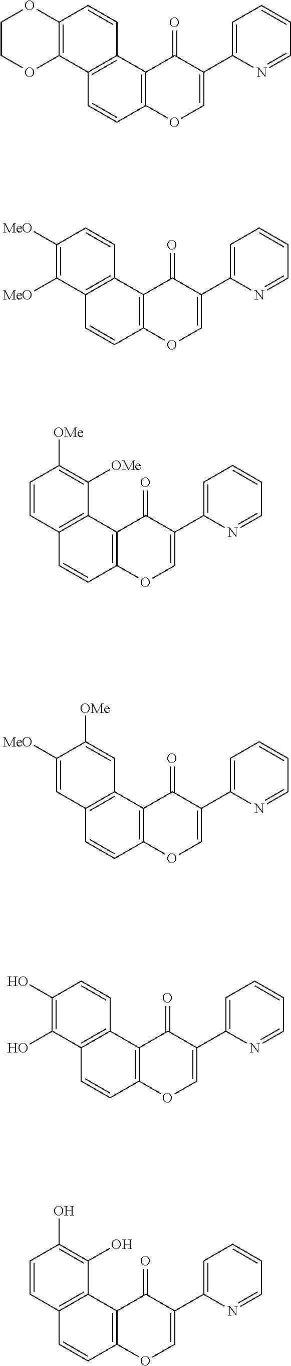 [SMILES: O=c1c(-c2ccccn2)coc2ccc3c4c(ccc3c12)OCCO4 ; COc1ccc2c(ccc3occ(-c4ccccn4)c(=O)c32)c1OC ; COc1ccc2ccc3occ(-c4ccccn4)c(=O)c3c2c1OC ; COc1cc2ccc3occ(-c4ccccn4)c(=O)c3c2cc1OC ; O=c1c(-c2ccccn2)coc2ccc3c(O)c(O)ccc3c12 ; O=c1c(-c2ccccn2)coc2ccc3ccc(O)c(O)c3c12]

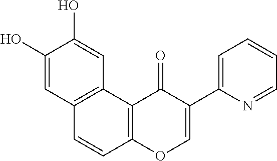 [SMILES: O=c1c(-c2ccccn2)coc2ccc3cc(O)c(O)cc3c12]

or a pharmaceutically acceptable salt, ester or prodrug form thereof.

In certain other embodiments, the compound is one of the following:

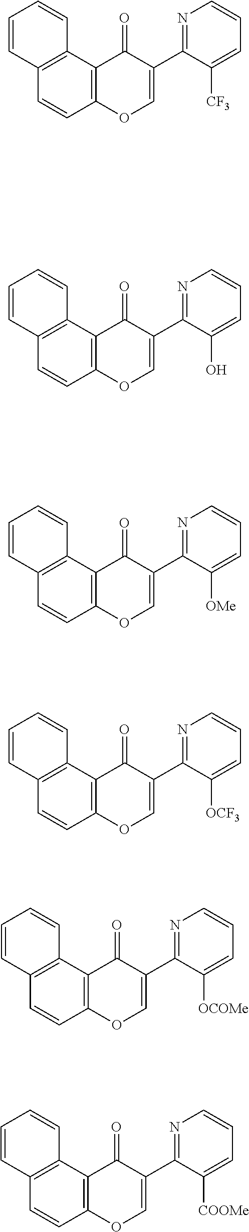

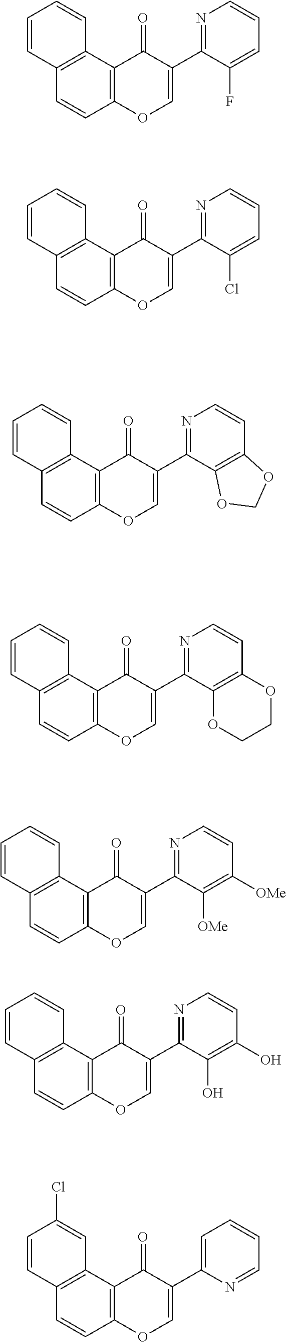

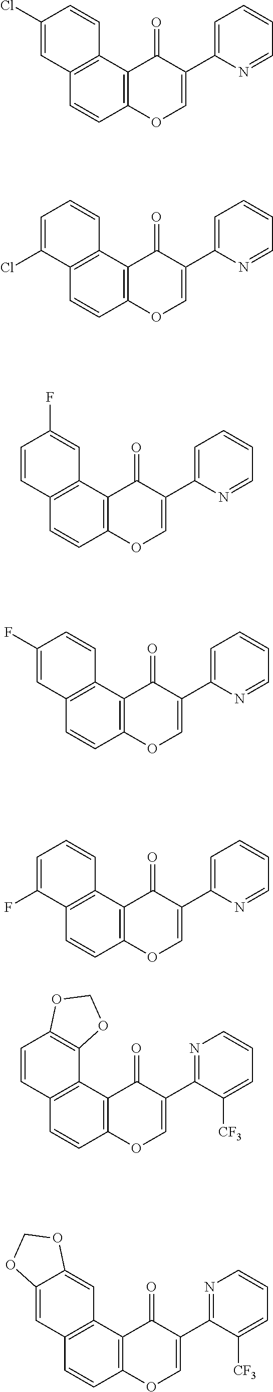

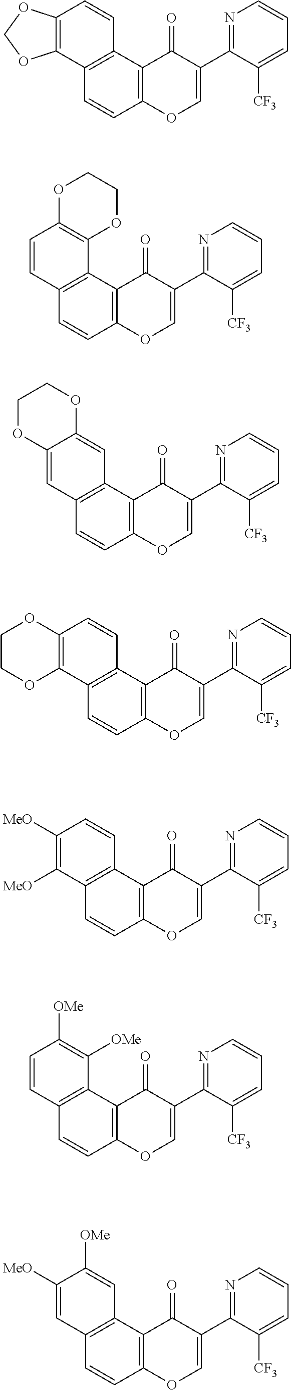

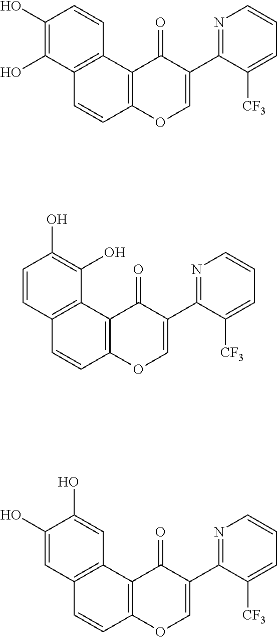 [SMILES: O=c1c(-c2ncccc2C(F)(F)F)coc2ccc3c(O)c(O)ccc3c12 ; O=c1c(-c2ncccc2C(F)(F)F)coc2ccc3ccc(O)c(O)c3c12 ; O=c1c(-c2ncccc2C(F)(F)F)coc2ccc3cc(O)c(O)cc3c12]

or a pharmaceutically acceptable salt, ester or prodrug form thereof.

In some embodiments, an asymmetric center is present in the compound, and the compound is a racemic mixture, a diastereoisomeric mixture, a single enantiomer, an enantiomeric diastereomer, a meso compound, a pure epimer, or a mixture of epimers thereof.

In other embodiments, one or more double bonds present in the compound are cis or trans, E or Z, a cis/trans mixture, an E/Z mixture, a combination of E and Z geometries, a combination of E and Z geometric mixtures or other geometric isomers thereof.

In some embodiments, the compound is present in the composition at a concentration of between about 0.005% and about 5% by weight.

The present invention also provides a pharmaceutical composition comprising a pharmaceutically acceptable carrier and a compound or of a pharmaceutically acceptable salt of the compound, wherein the compound has the structure:

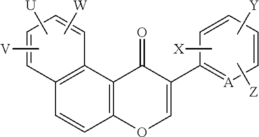

wherein:

each of U, V, W, X, Y, and Z is independently:

H; OH; F; Cl; Br; I; C₁ to C6 straight chain or branched chain alkyl; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO3H₂ or a phosphate thereof; PO₂(OCH₃)H or a phosphonate thereof; NO₂; NH₂; NHCH(O); NRCH(O); NHC(O)R; NRC(O)R₁; C(O)NRR₁; C(NH)NRR₁; C(NH)NROH; C(NH)NRNO2; or C(NR)NR₁C(NR₂)NR₃R₄;

wherein adjacent substituents U, V and W or X, Y and Z may form a saturated or unsaturated 5-membered or 6-membered carbocyclic or heterocyclic ring;

wherein each of R, R₁, R₂, R₃ and R₄ if present is independently:

H; OH; O-Rx; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

wherein Rx, if present, is alkyl, cycloalkyl, alkylcycloalkyl, acyl, ester or thioester;

and wherein A is CH or N.

In some embodiments, adjacent substituents U, V and W and X, Y and Z may form a saturated or unsaturated 5-membered or 6-membered carbocyclic or heterocyclic ring.

In further embodiments, each of U, V, W, X, Y, and Z is independently: H; OH; F; Cl; Br; I; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a phosphate thereof; PO₂(OCH₃)H or a phosphonate thereof; NH₂; NHCH(O); NRCH(O); NHC(O)R; NRC(O)R₁; C(O)NRR₁; C(NH)NRR₁; C(NH)NROH; or C(NR)NR₁C(NR₂)NR₃R₄;

each of R, R₁, R₂, R₃ and R₄ If present is independently: H, OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

and wherein A is CH or N.

In further embodiments, each of U, V, W, X, Y, and Z is independently:

H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR)—R; CO₂H or an ester thereof; NH₂; NHCH(O); NRCH(O); NHC(O)R; NRC(O)R₁; C(O)NRR₁;or C(NH)NRR₁;

each of R and R₁ if present is independently: H; OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

and wherein A is CH or N.

In other embodiments, each of U, V, W, X, Y, and 2 is independently:

H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;

each of R and R₁ if present is independently: H; OH; optionally substituted alkyl; cycloalkyl; alkylcycloalkyl; heterocycloalkyl; alkylheterocycloalkyl; optionally substituted alkenyl; or optionally substituted alkynyl;

and wherein A is CH or N.

In further embodiments, at least one of U, V and W is H and at least one of X, Y and Z is H.

In other embodiments, at least two of U, V and W is H or at least two of X, Y and Z is H.

In yet other embodiments, each of U, V and W is H or each of X, Y and Z is H.

In additional embodiments, one of U, V and W is H and each of X, Y and Z is H; one of X, Y and Z is H and each of U, V and W is H; two of U, V and W is H and each of X, V and Z is H; or two of X, Y and Z is H and each of U, V and W is H.

In yet further embodiments, at least one of U, V W, X, Y and Z is other than H.

In certain embodiments, the compound is one of the following:

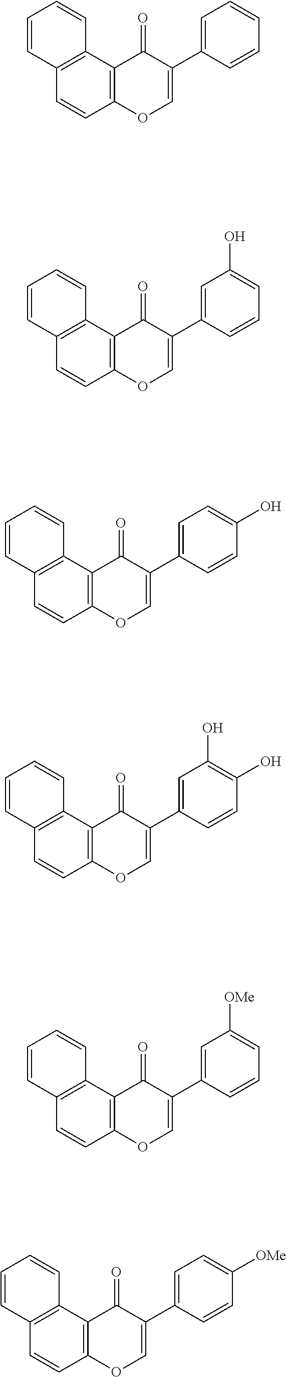 [SMILES: O=c1c(-c2ccccc2)coc2ccc3ccccc3c12 ; O=c1c(-c2cccc(O)c2)coc2ccc3ccccc3c12 ; O=c1c(-c2ccc(O)cc2)coc2ccc3ccccc3c12 ; O=c1c(-c2ccc(O)c(O)c2)coc2ccc3ccccc3c12 ; COc1cccc(-c2coc3ccc4ccccc4c3c2=O)c1 ; COc1ccc(-c2coc3ccc4ccccc4c3c2=O)cc1]

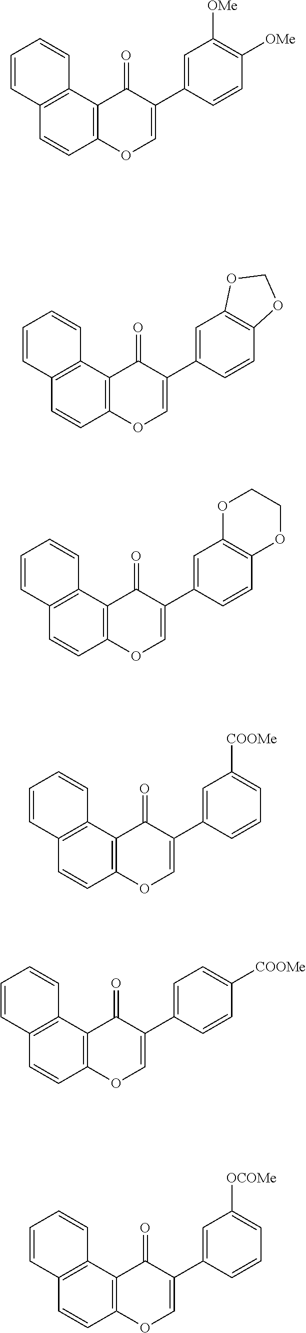

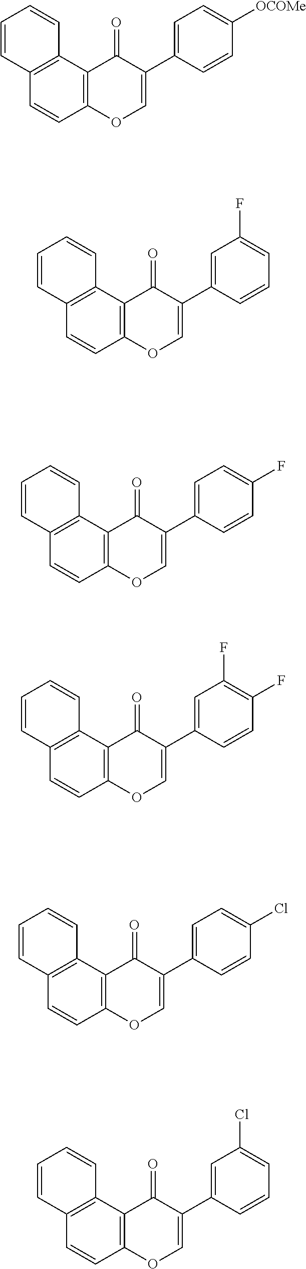

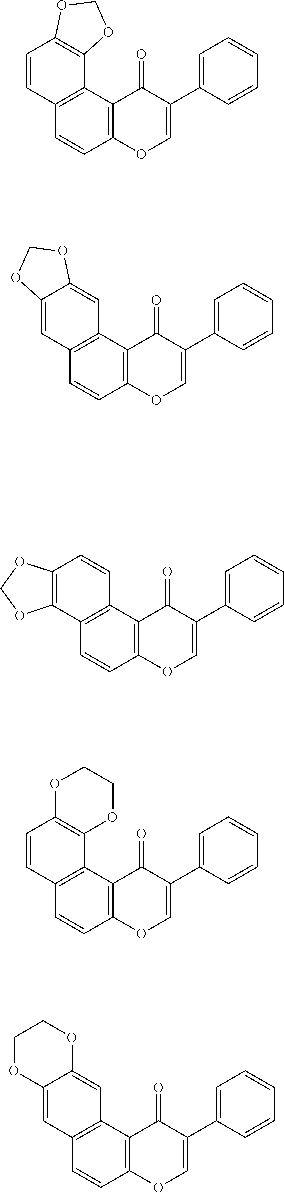 [SMILES: O=c1c(-c2ccccc2)coc2ccc3ccc4c(c3c12)OCO4 ; O=c1c(-c2ccccc2)coc2ccc3cc4c(cc3c12)OCO4 ; O=c1c(-c2ccccc2)coc2ccc3c4c(ccc3c12)OCO4 ; O=c1c(-c2ccccc2)coc2ccc3ccc4c(c3c12)OCCO4 ; O=c1c(-c2ccccc2)coc2ccc3cc4c(cc3c12)OCCO4]

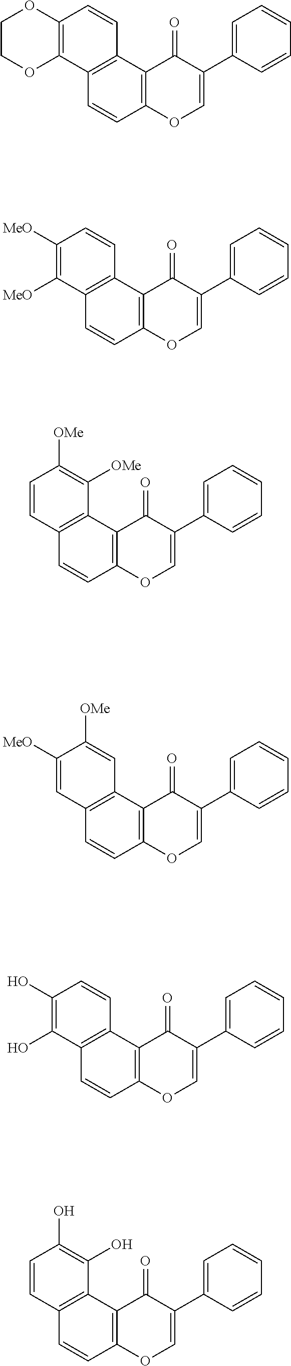 [SMILES: O=c1c(-c2ccccc2)coc2ccc3c4c(ccc3c12)OCCO4 ; COc1ccc2c(ccc3occ(-c4ccccc4)c(=O)c32)c1OC ; COc1ccc2ccc3occ(-c4ccccc4)c(=O)c3c2c1OC ; COc1cc2ccc3occ(-c4ccccc4)c(=O)c3c2cc1OC ; O=c1c(-c2ccccc2)coc2ccc3c(O)c(O)ccc3c12 ; O=c1c(-c2ccccc2)coc2ccc3ccc(O)c(O)c3c12]

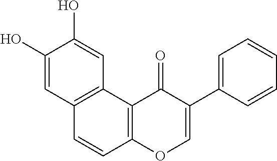 [SMILES: O=c1c(-c2ccccc2)coc2ccc3cc(O)c(O)cc3c12]

or a pharmaceutically acceptable salt, ester or prodrug form thereof.

In certain other embodiments, the compound is one of the following:

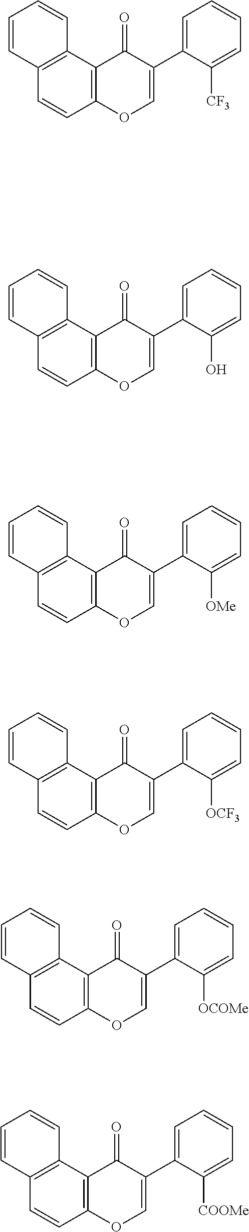

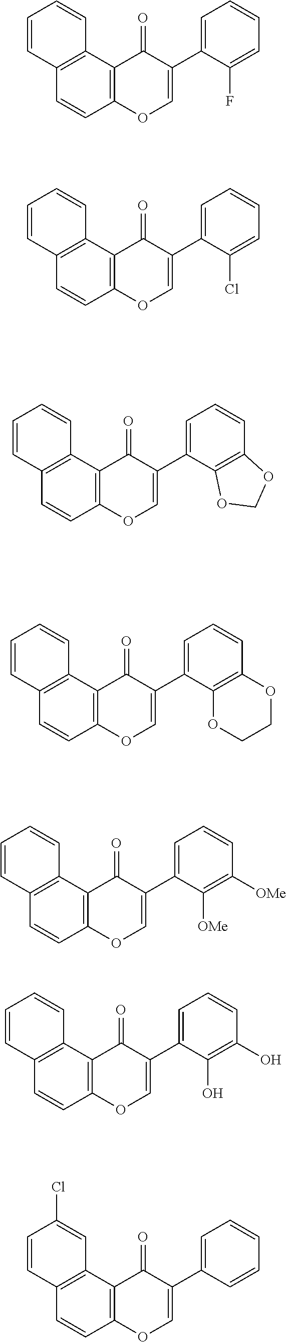

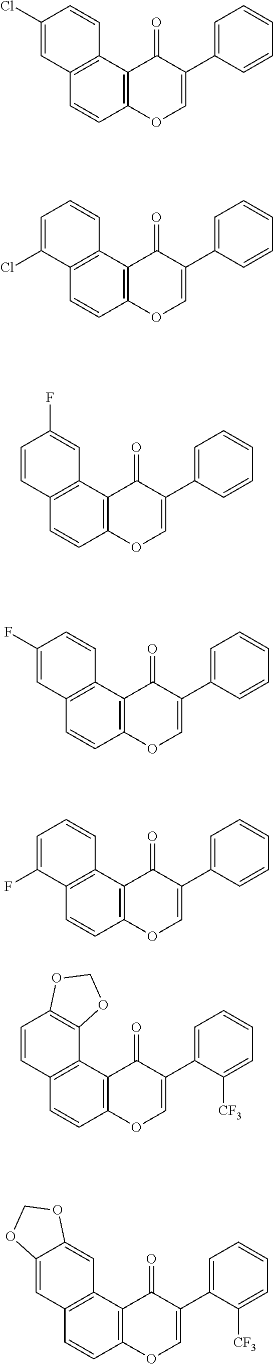

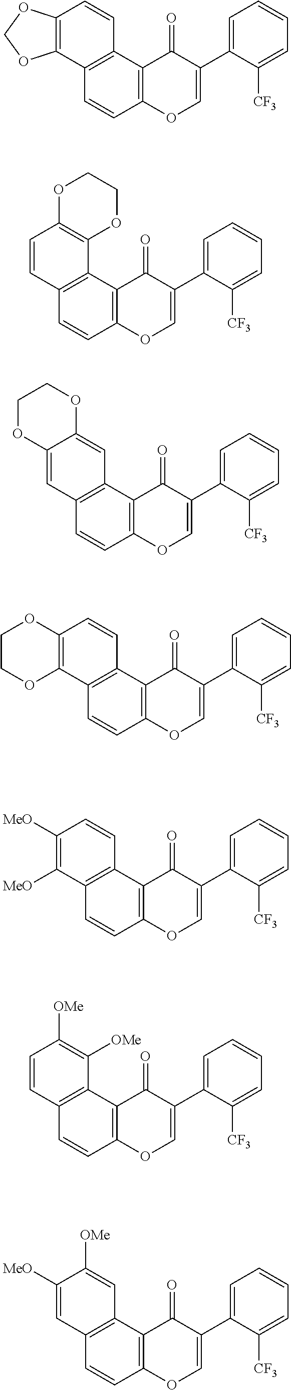

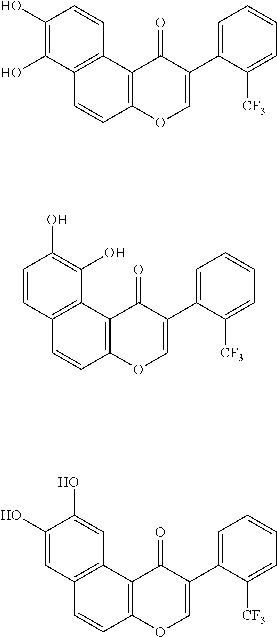 [SMILES: O=c1c(-c2ccccc2C(F)(F)F)coc2ccc3c(O)c(O)ccc3c12 ; O=c1c(-c2ccccc2C(F)(F)F)coc2ccc3ccc(O)c(O)c3c12 ; O=c1c(-c2ccccc2C(F)(F)F)coc2ccc3cc(O)c(O)cc3c12]

or a pharmaceutically acceptable salt, ester or prodrug form thereof.

In certain other embodiments, the compound is one of the following:

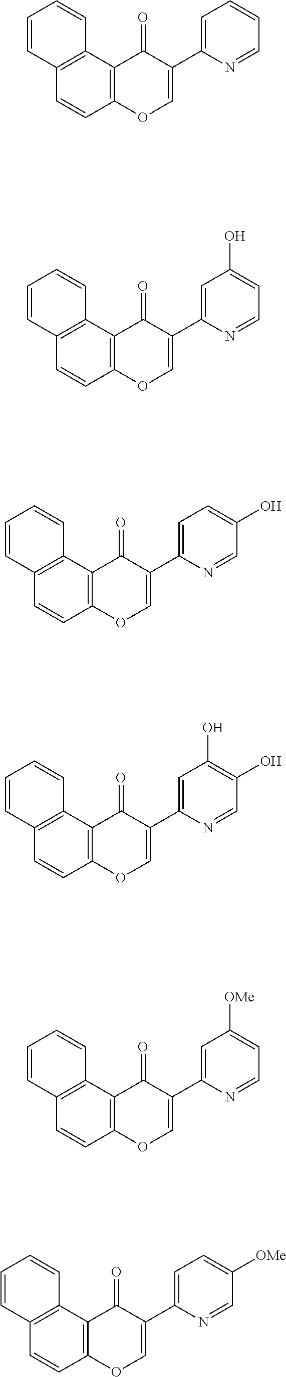 [SMILES: O=c1c(-c2ccccn2)coc2ccc3ccccc3c12 ; O=c1c(-c2cc(O)ccn2)coc2ccc3ccccc3c12 ; O=c1c(-c2ccc(O)cn2)coc2ccc3ccccc3c12 ; O=c1c(-c2cc(O)c(O)cn2)coc2ccc3ccccc3c12 ; COc1ccnc(-c2coc3ccc4ccccc4c3c2=O)c1 ; COc1ccc(-c2coc3ccc4ccccc4c3c2=O)nc1]

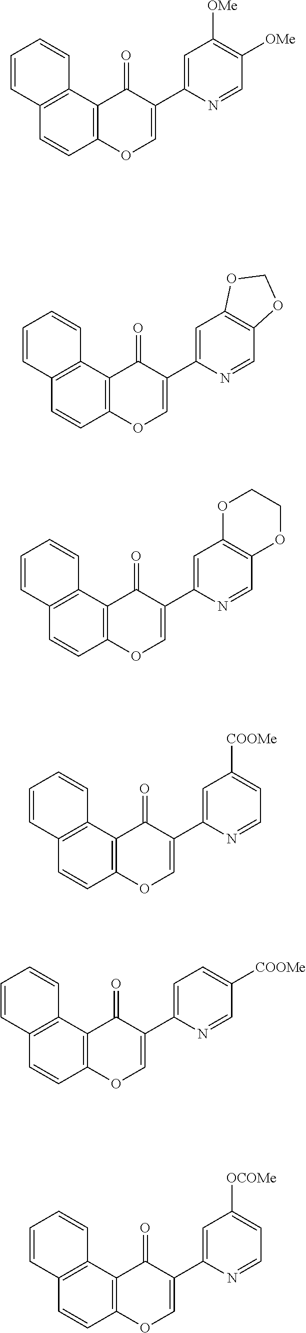

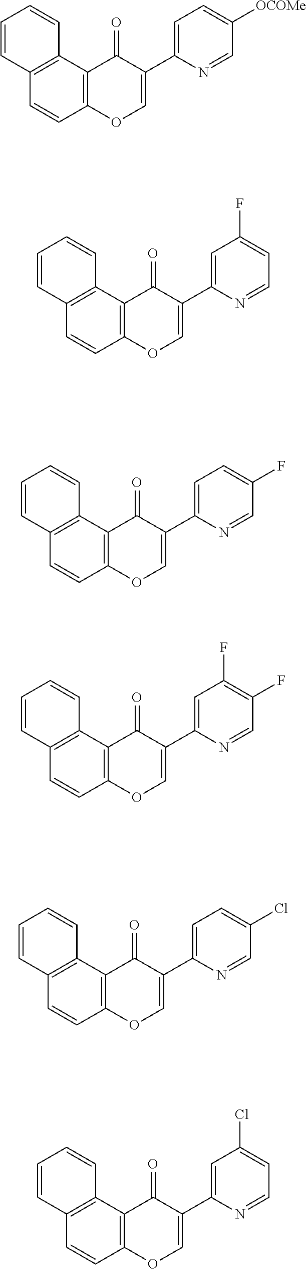

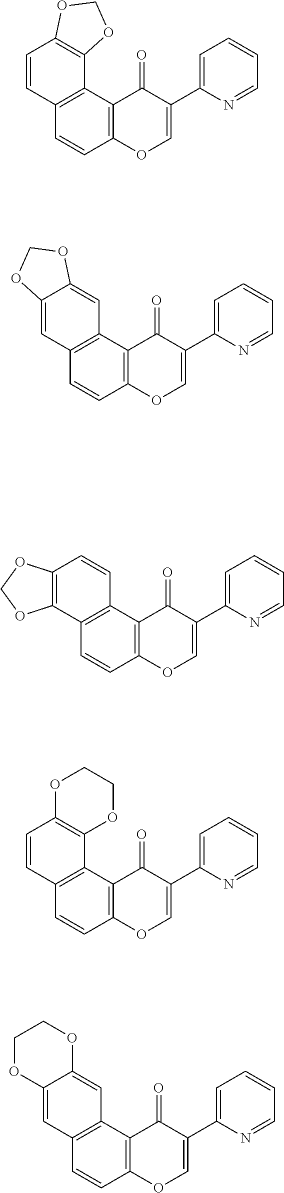 [SMILES: O=c1c(-c2ccccn2)coc2ccc3ccc4c(c3c12)OCO4 ; O=c1c(-c2ccccn2)coc2ccc3cc4c(cc3c12)OCO4 ; O=c1c(-c2ccccn2)coc2ccc3c4c(ccc3c12)OCO4 ; O=c1c(-c2ccccn2)coc2ccc3ccc4c(c3c12)OCCO4 ; O=c1c(-c2ccccn2)coc2ccc3cc4c(cc3c12)OCCO4]

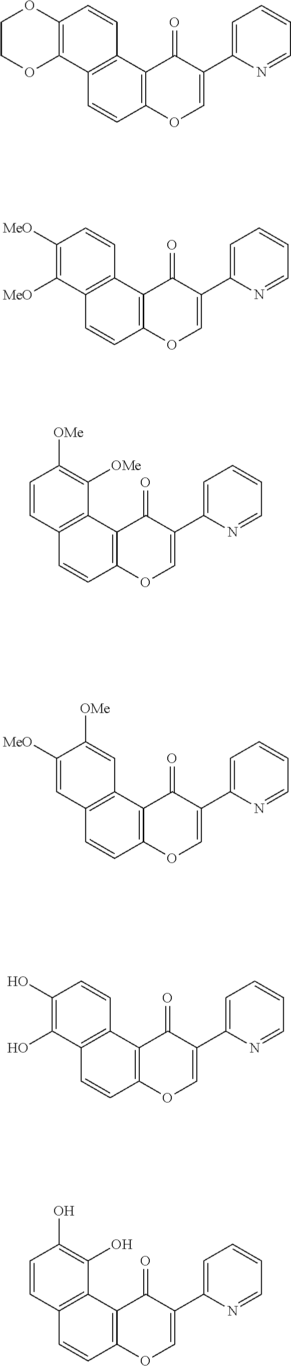 [SMILES: O=c1c(-c2ccccn2)coc2ccc3c4c(ccc3c12)OCCO4 ; COc1ccc2c(ccc3occ(-c4ccccn4)c(=O)c32)c1OC ; COc1ccc2ccc3occ(-c4ccccn4)c(=O)c3c2c1OC ; COc1cc2ccc3occ(-c4ccccn4)c(=O)c3c2cc1OC ; O=c1c(-c2ccccn2)coc2ccc3c(O)c(O)ccc3c12 ; O=c1c(-c2ccccn2)coc2ccc3ccc(O)c(O)c3c12]

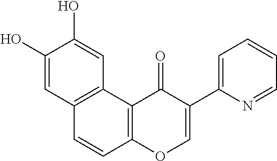 [SMILES: O=c1c(-c2ccccn2)coc2ccc3cc(O)c(O)cc3c12]

or a pharmaceutically acceptable salt, ester or prodrug form thereof.

In certain other embodiments, the compound is one of the following:

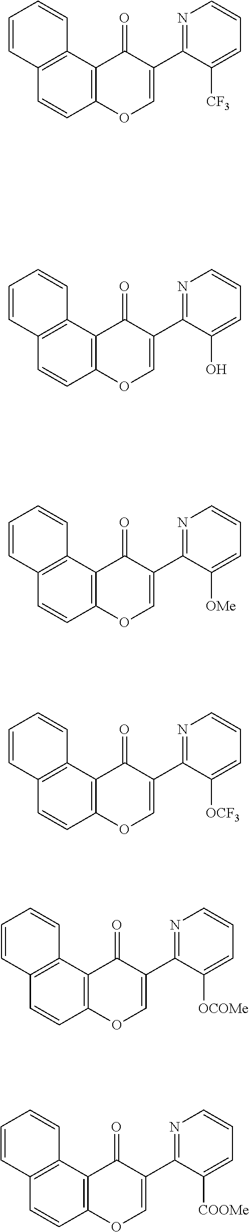

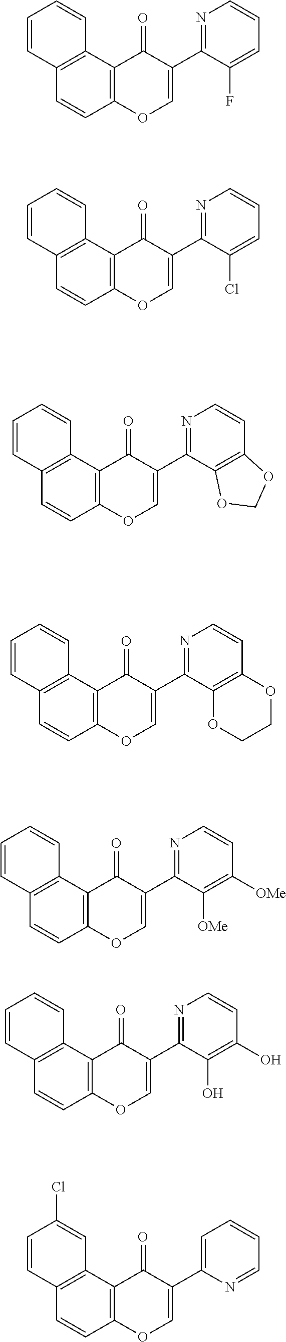

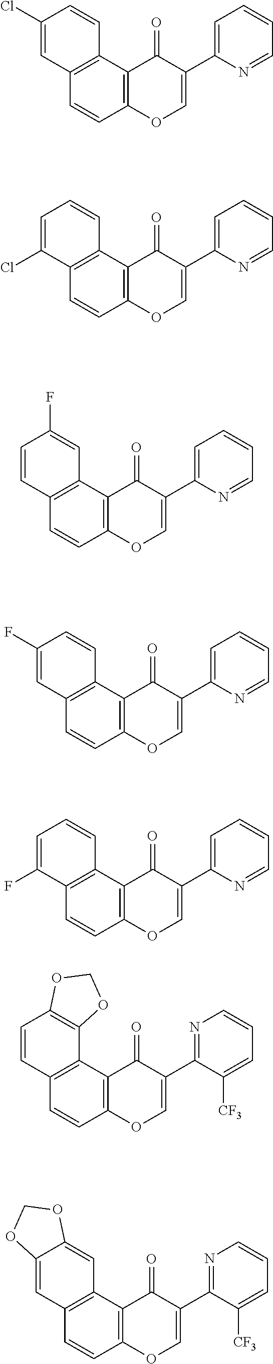

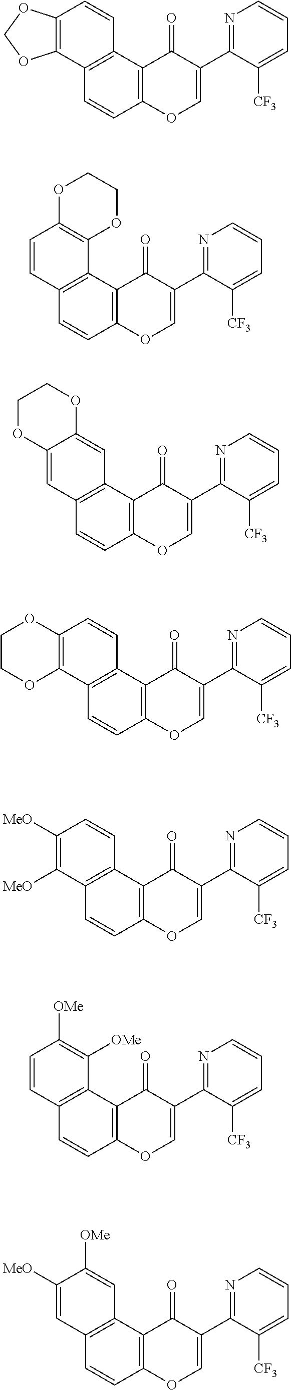

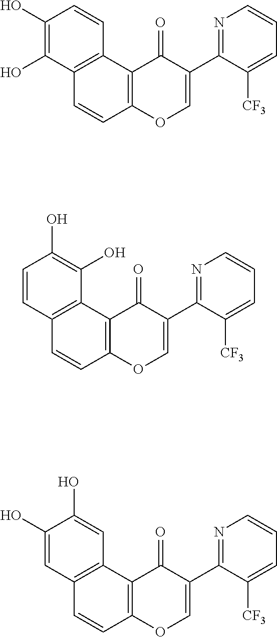 [SMILES: O=c1c(-c2ncccc2C(F)(F)F)coc2ccc3c(O)c(O)ccc3c12 ; O=c1c(-c2ncccc2C(F)(F)F)coc2ccc3ccc(O)c(O)c3c12 ; O=c1c(-c2ncccc2C(F)(F)F)coc2ccc3cc(O)c(O)cc3c12]

or a pharmaceutically acceptable salt, ester or prodrug form thereof.

In other embodiments, an asymmetric center Is present in the compound, and the compound is a racemic mixture, a diastereoisomeric mixture, a single enantiomer, an enantiomeric diastereomer, a meso compound, a pure epimer, or a mixture of epimers thereof.

In yet other embodiments, one or more double bonds present in the compound are cis or trans, E or Z, a cis/trans mixture, an E/2 mixture, a combination of E and Z geometries, a combination of E and Z geometric mixtures or other geometric isomers thereof.

In some embodiments, the compound is present in the pharmaceutical composition at a concentration between about 0.005% and about 5%.

In another embodiment, the pharmaceutical composition further comprises a second therapeutic agent.

In some embodiments, the compound has a lipophilicity as measured by LogP of greater than 3.

In further embodiments, the compound, or pharmaceutically acceptable salt thereof, is suitable for topical use. In other embodiments, the compound, or a pharmaceutically acceptable salt thereof, is suitable for intralesional use.

The present invention yet further provides a compound having the structure I, or a pharmaceutically acceptable salt thereof,

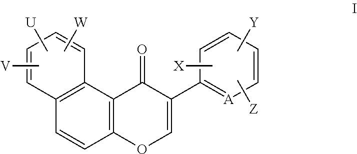

wherein:

each of U, V, W, X, Y, and 2 is independently:

H; OH; F; Cl; Br; I; C₁ to C6 straight chain or branched chain alkyl; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a phosphate thereof; PO₂(OCH₃)H or a phosphonate thereof; NO₂; NH₂; NHCH(O); NRCH(O); NHC(O)R; NRC(O)R₁; C(O)NRR₁; C(NH)NRR₁; C(NH)NROH; C(NH)NRNO2; or C(NR)NR₁C(NR₂)NR₃R₄;

wherein adjacent substituents U, V and W or X, Y and Z may form a saturated or unsaturated 5-membered or 6-membered carbocyclic or heterocyclic ring;

wherein each of R, R₁, R₂, R₃ and R₄ if present is independently:

H; OH; O-Rx; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

wherein Rx, if present, is alkyl, cycloalkyl, alkylcycloalkyl, acyl, ester or thioester;

and wherein A is CH or N.

In some embodiments, adjacent substituents U, V and W and X, Y and Z may form a saturated or unsaturated 5-membered or 6-membered carbocyclic or heterocyclic ring.

In further embodiments, each of U, V, W, X, Y, and Z is independently: H; OH; F; Cl; Br; I; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNRO-R; SO₃H or an ester thereof; CO₂H or an ester thereof; PO₃H₂ or a phosphate thereof; PO₂(OCH₃)H or a phosphonate thereof; NH₂; NHCH(O); NRCH(O); NHC(O)R; NRC(O)R₁; C(O)NRR₁C(NH)NRR₁; C(NH)NROH; or C(NR)NR₁C(NR₂)NR₃R₄;

each of R, R₁, R₂, R₃ and R₄ If present is independently: H, OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

and wherein A is CH or N.

In further embodiments, each of U, V, W, X, Y, and Z is independently:

H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;

each of R and R₁ if present is independently: H; OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;

and wherein A is CH or N.

In other embodiments, each of U, V, W, X, Y, and Z is independently:

H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;

each of R and R₁ if present is independently: H; OH; optionally substituted alkyl; cycloalkyl; alkylcycloalkyl; heterocycloalkyl; alkylheterocycloalkyl; optionally substituted alkenyl; or optionally substituted alkynyl;

and wherein A is CH or N.

In further embodiments, at least one of U, V and W is H and at least one of X, Y and Z is H.

In other embodiments, at least two of U, V and W is H or at least two of X, Y and Z is H.

In yet other embodiments, each of U, V and W is H or each of X, Y and Z is H.

In additional embodiments, one of U, V and W Is H and each of X, Y and Z is H; one of X, Y and Z is H and each of U, V and W is H; two of U, V and W is H and each of X, Y and Z is H; or two of X, Y and Z is H and each of U, V and W is H.

In yet further embodiments, at least one of U, V W, X, Y and Z is other than H.

In certain embodiments, the compound is one of the following:

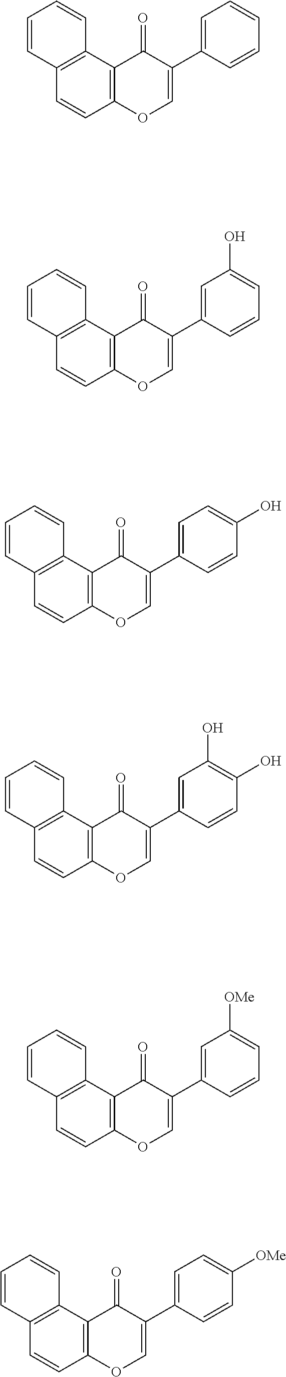 [SMILES: O=c1c(-c2ccccc2)coc2ccc3ccccc3c12 ; O=c1c(-c2cccc(O)c2)coc2ccc3ccccc3c12 ; O=c1c(-c2ccc(O)cc2)coc2ccc3ccccc3c12 ; O=c1c(-c2ccc(O)c(O)c2)coc2ccc3ccccc3c12 ; COc1cccc(-c2coc3ccc4ccccc4c3c2=O)c1 ; COc1ccc(-c2coc3ccc4ccccc4c3c2=O)cc1]

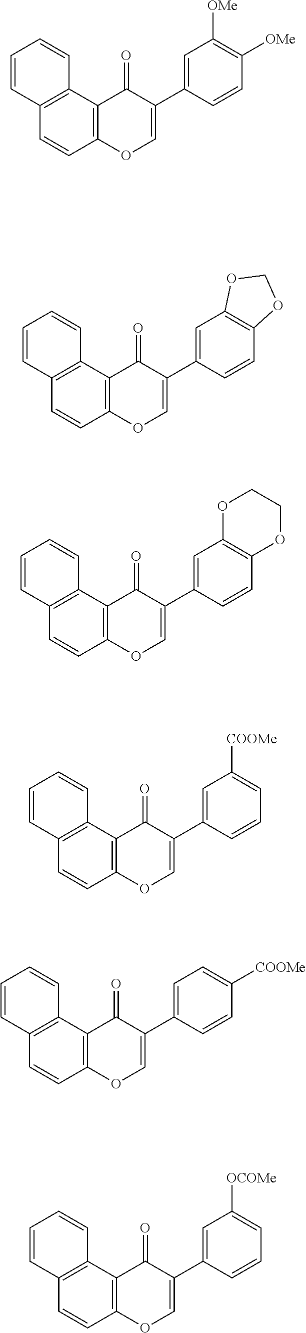

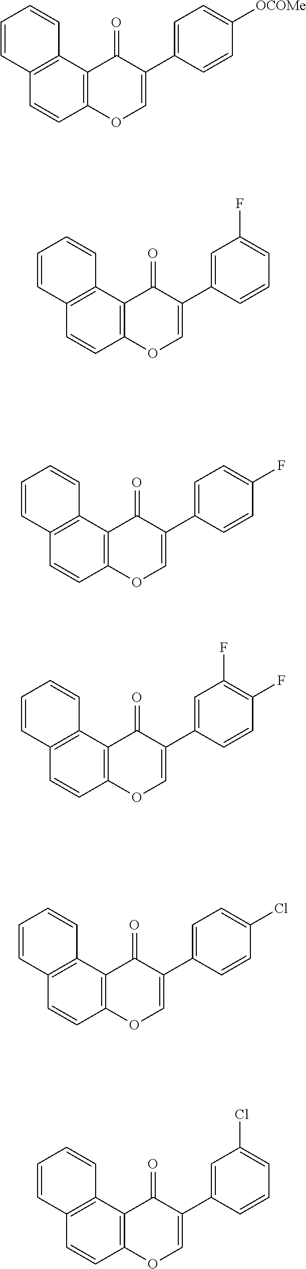

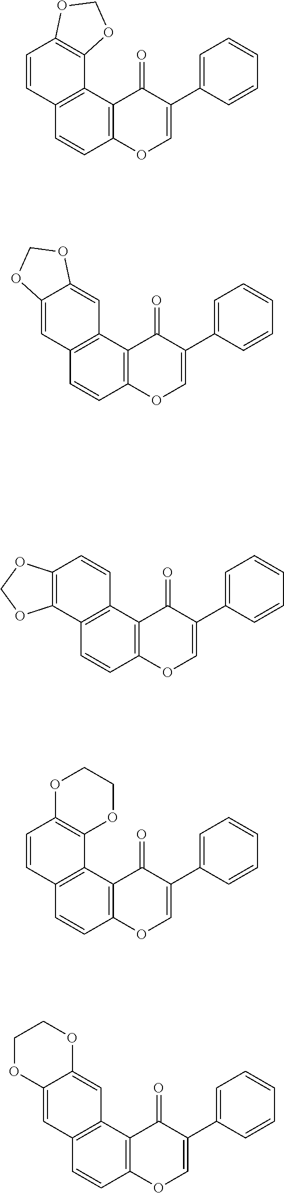 [SMILES: O=c1c(-c2ccccc2)coc2ccc3ccc4c(c3c12)OCO4 ; O=c1c(-c2ccccc2)coc2ccc3cc4c(cc3c12)OCO4 ; O=c1c(-c2ccccc2)coc2ccc3c4c(ccc3c12)OCO4 ; O=c1c(-c2ccccc2)coc2ccc3ccc4c(c3c12)OCCO4 ; O=c1c(-c2ccccc2)coc2ccc3cc4c(cc3c12)OCCO4]

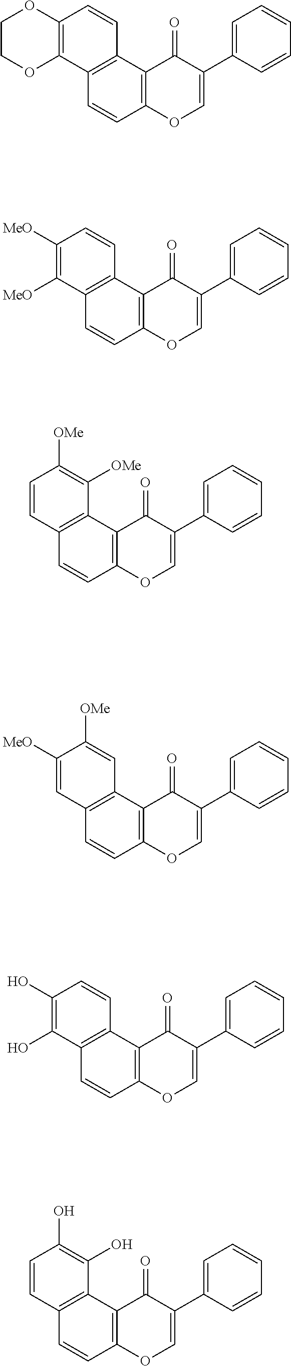 [SMILES: O=c1c(-c2ccccc2)coc2ccc3c4c(ccc3c12)OCCO4 ; COc1ccc2c(ccc3occ(-c4ccccc4)c(=O)c32)c1OC ; COc1ccc2ccc3occ(-c4ccccc4)c(=O)c3c2c1OC ; COc1cc2ccc3occ(-c4ccccc4)c(=O)c3c2cc1OC ; O=c1c(-c2ccccc2)coc2ccc3c(O)c(O)ccc3c12 ; O=c1c(-c2ccccc2)coc2ccc3ccc(O)c(O)c3c12]

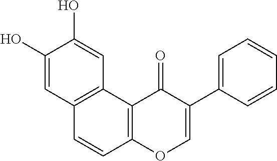 [SMILES: O=c1c(-c2ccccc2)coc2ccc3cc(O)c(O)cc3c12]

or a pharmaceutically acceptable salt, ester or prodrug form thereof.

In certain other embodiments, the compound is one of the following:

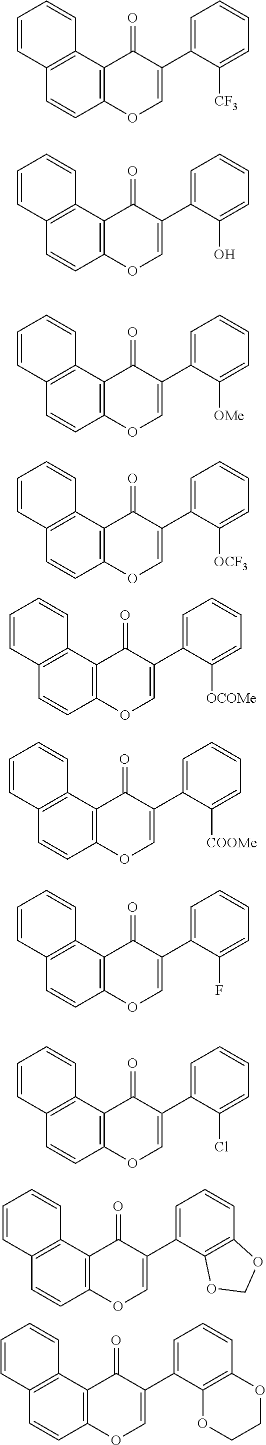

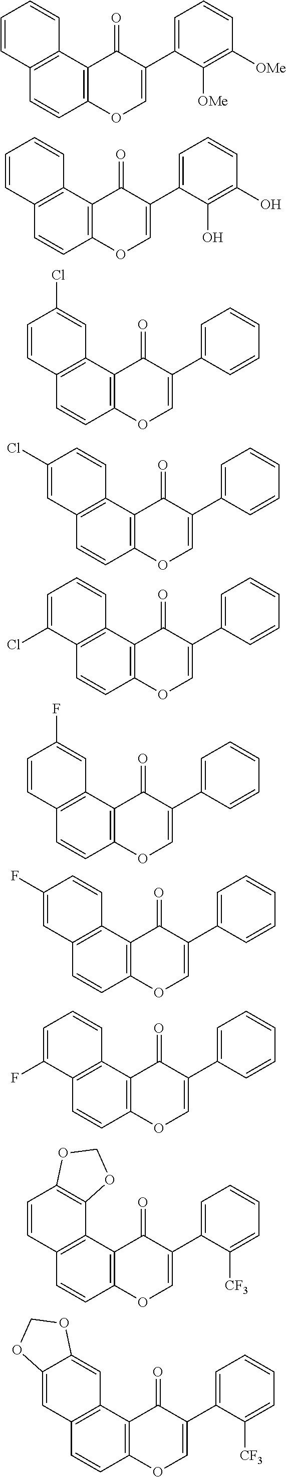

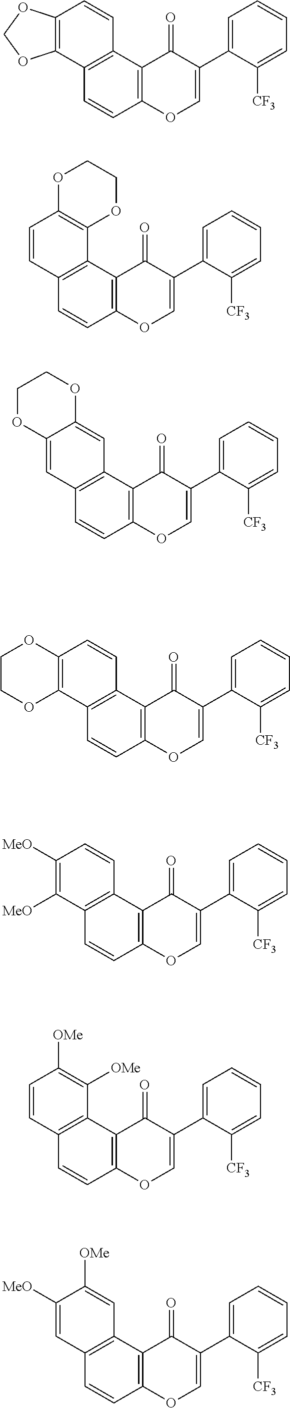

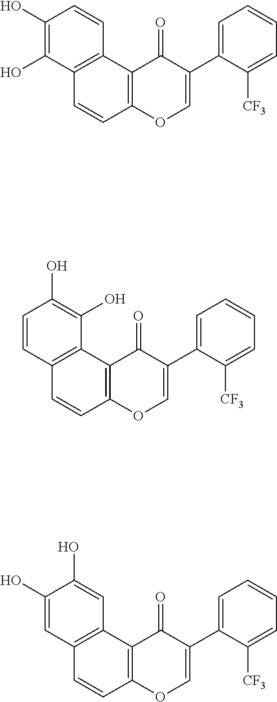 [SMILES: O=c1c(-c2ccccc2C(F)(F)F)coc2ccc3c(O)c(O)ccc3c12 ; O=c1c(-c2ccccc2C(F)(F)F)coc2ccc3ccc(O)c(O)c3c12 ; O=c1c(-c2ccccc2C(F)(F)F)coc2ccc3cc(O)c(O)cc3c12]

or a pharmaceutically acceptable salt, ester or prodrug form thereof.

In certain other embodiments, the compound is one of the following:

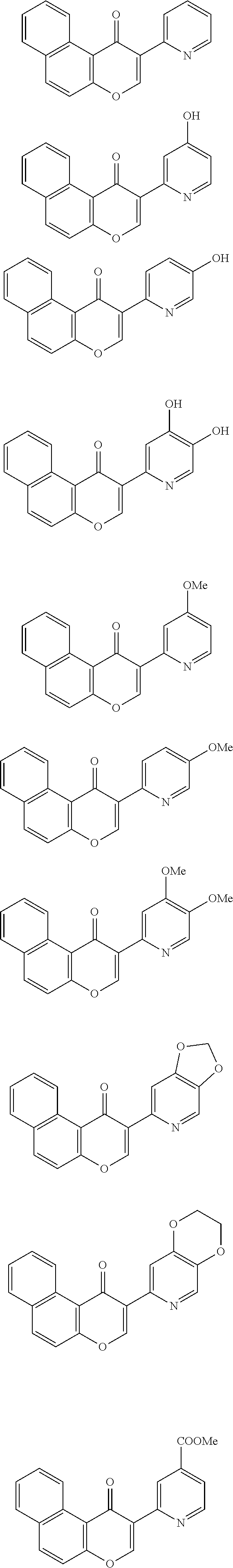

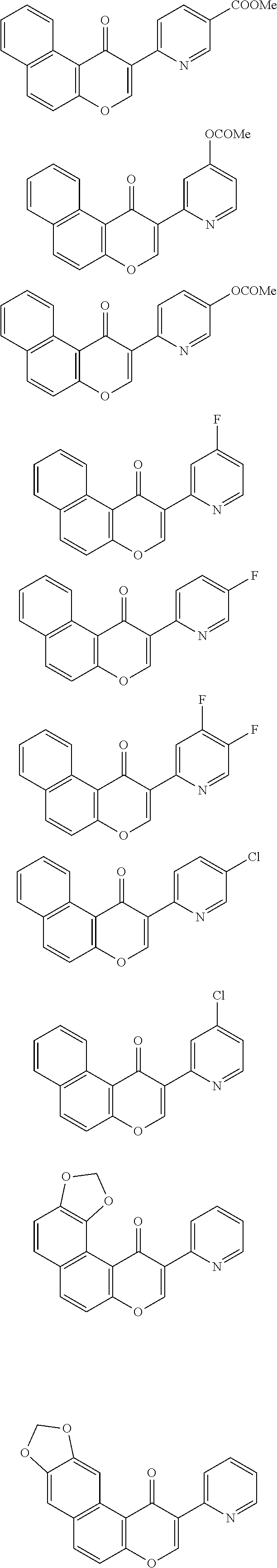

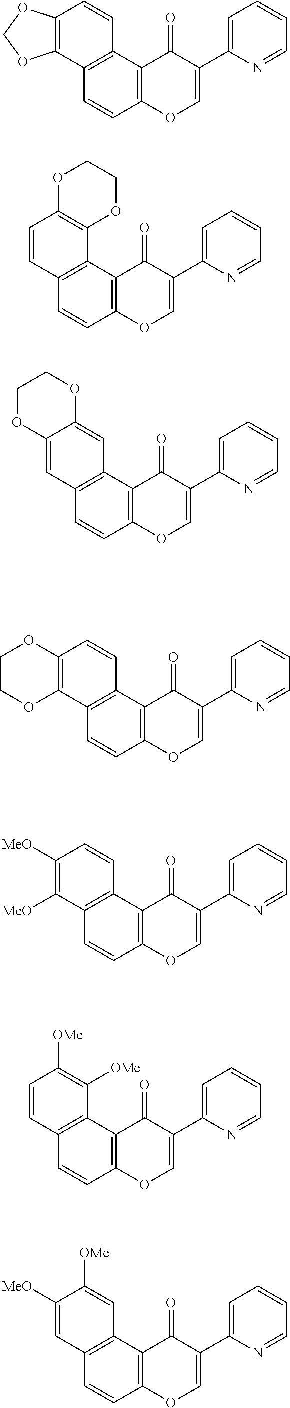

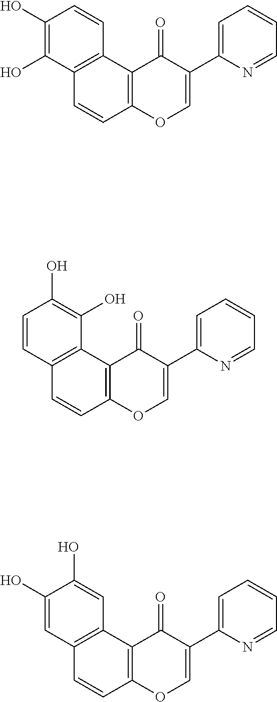 [SMILES: O=c1c(-c2ccccn2)coc2ccc3c(O)c(O)ccc3c12 ; O=c1c(-c2ccccn2)coc2ccc3ccc(O)c(O)c3c12 ; O=c1c(-c2ccccn2)coc2ccc3cc(O)c(O)cc3c12]

or a pharmaceutically acceptable salt, ester or prodrug form thereof.

In certain other embodiments, the compound is one of the following:

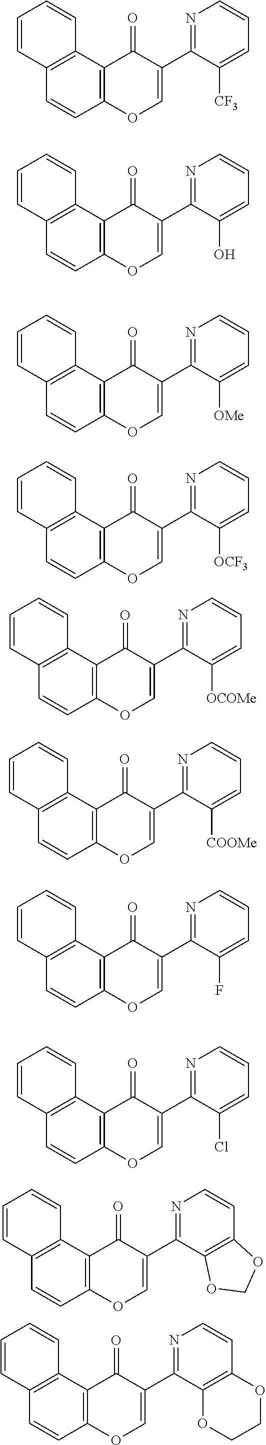

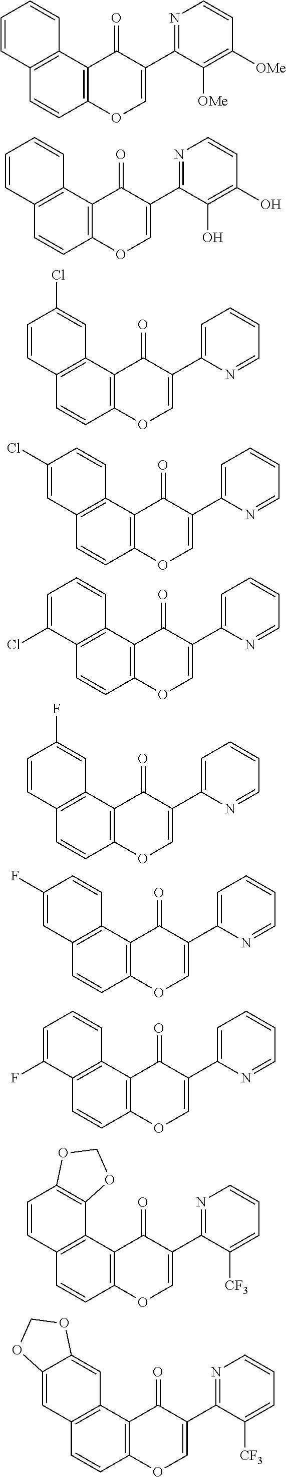

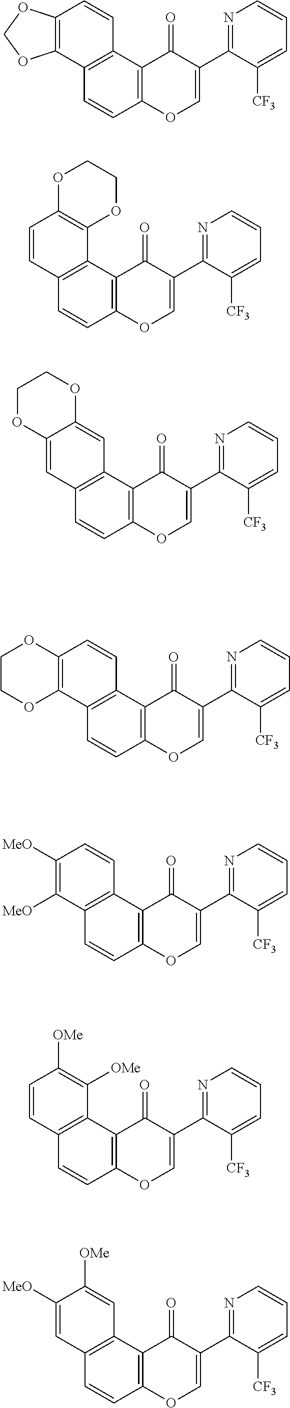

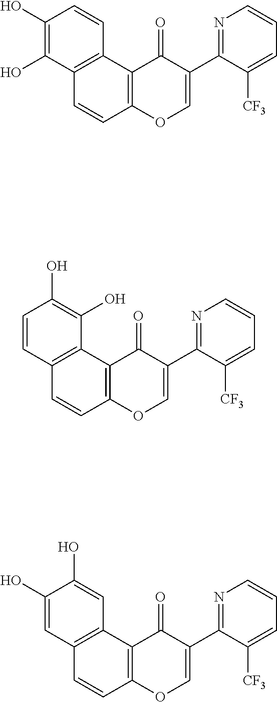 [SMILES: O=c1c(-c2ncccc2C(F)(F)F)coc2ccc3c(O)c(O)ccc3c12 ; O=c1c(-c2ncccc2C(F)(F)F)coc2ccc3ccc(O)c(O)c3c12 ; O=c1c(-c2ncccc2C(F)(F)F)coc2ccc3cc(O)c(O)cc3c12]

or a pharmaceutically acceptable salt, ester or prodrug form thereof.

In some embodiments, an asymmetric center is present in the compound, and the compound is a racemic mixture, a diastereoisomeric mixture, a single enantiomer, an enantiomeric diastereomer, a mew compound, a pure epimer, or a mixture of epimers thereof.

In other embodiments, one or more double bonds present in the compound are cis or trans, E or Z, a cis/trans mixture, an E/Z mixture, a combination of E and Z geometries, a combination of E and Z geometric mixtures or other geometric isomers thereof.

In some embodiments, the compound has a lipophilicity as measured by LogP of greater than 3.

In further embodiments, the compound or a pharmaceutically acceptable salt thereof are suitable for topical use.

In some embodiments, the compound having the structure I has improvements to one or several relevant drug-like properties compared to 3-phenyl-111-benzo[f]chromen-1-one such as greater potency, optimization of solubility, optimization of hydrophobicity, optimization of half-life etc.

In some embodiments, the compound of the present invention may also have one or several of the following properties:
a) an ability to activate the AhR receptor,
b) an ability to modulate a gene regulated by AhR,
c) a short half-life in the human organism of between 0 hours and 96 h, and
d) a measurable positive effect on a recognized criterion of sebaceous hyperactivity.

In yet further embodiments, the compound of the present invention may also have one or several of the following properties:
a) an ability to down regulate the expression of genes involved in the synthesis of lipids in sebum,
b) a short half-life in the human organism of between 0 hours and 96 h, and
c) a measurable positive effect on a recognized criterion of sebaceous hyperactivity.

In another embodiment, the compound has an ability to down regulate the expression of anti-inflammatory genes such as ALOX-15. In a separate embodiment, the compound has an ability to up regulate the expression of anti-inflammatory genes such as ALOX-15.

The present invention also provides a method of (i) activating the AhR receptor, (ii) modulating a gene regulated by AhR, (iii) down regulating the expressing of genes involved in the synthesis of lipids in sebum and/or (iv) modulating one or several enzymes involved in lipid production, comprising contacting the compound having the structure I with the AhR receptor. In a further embodiment, the AhR receptor Is in a subject and the compound is administered to the subject.

In further embodiments, compounds of Structure I and pharmaceutical compositions thereof are activators of Aryl Hydrocarbon receptor pathways, resulting in significant reduction of both sebum and cholesterol esters. In other embodiments the compounds of the present invention and pharmaceutical compositions thereof are useful for the treatment of skin conditions or diseases mediated by Aryl Hydrocarbon receptor pathways.

In further embodiments, the pharmaceutical compositions of the present invention comprises a therapeutically effective amount of a compound which has the structure I, or a pharmaceutically acceptable salt, solvate or hydrate of the compound and a pharmaceutically acceptable carrier.

In some embodiments, the skin condition associated with abnormal sebum secretion or abnormal sebaceous gland function is associated with estrogen deprivation.

In yet other embodiments, the compounds encompassed by Structure I and pharmaceutical compositions thereof are also useful for decreasing the amount of sebum produced and/or secreted by the sebaceous glands of a human subject.

In some embodiments, the compounds are administered topically, locally e.g., by intra-dermal injection, subcutaneously or intralesionally, or orally.

In further embodiments, compounds of structure I are useful for fat reduction (sub-mental or other areas), body sculpting, treatment of lipomas, liposarcomas and cellulite reduction.

In alternative embodiments, the compounds and pharmaceutical compositions of the present invention may be administered in therapeutically effective amounts to treat disease such as, but not limited to, pain, inflammation, and neurodegenerative diseases, neuropathic pain, trigeminal neuralgia, postherpetic neuralgia, diabetic neuropathy, cancer pain, phantom limb pain, complex regional pain syndrome, and fibromyalgia; rheumatoid arthritis, ankolysing spondylitis, ulcerative colitis, tendonitis, psoriasis, Hidradenitis Suppurativa (sometimes referred to as Acne Inversa), Faber's Disease, Crohn's Disease, rhinitis, skin allergies, asthma, autoimmune diseases with inflammatory components such as multiple sclerosis and other demyelinating disorders; Alzheimer's Disease, traumatic brain injury, conditions and diseases characterized by abnormal ArH receptor activities, metabolic disorders, appetite regulation, and obesity.

The present invention further provides a method of treating allergic contact dermatitis, atopic dermatitis, seborrheic dermatitis, eczema, urticaria, rosacea, acne, psoriasis, Hidradenitis Suppurativa (sometimes referred to as Acne inversa), pruritus, lichen, psoriatic arthritis acne, scarring, skin wound healing, skin burns deriving from various origins, such as sunburns or radiation therapy burns, and of various severities (first degree burn, second degree burn, third degree burn, fourth degree burn), scleroderma, solar keratosis, squamous cell carcinoma, or melanoma.

In some embodiments, the compounds and pharmaceutical compositions, and methods of administering them are useful for treating inflammation.

The present invention yet further provides an article of manufacture or kit containing a therapeutically effective amount of compounds of Structure I, or a pharmaceutically acceptable salt thereof, or solvates or hydrates of said compounds or salts, packaged for retail distribution, in association with instructions advising the consumer on how to use the compound to alleviate a condition associated with excess sebum production and/or secretion.

The following sections provide additional non-limiting details of the compounds of Structure I, their compositions and uses. The headings within this document are only being utilized to expedite its review by the reader and should not be construed as limiting the invention or claims in any manner.

Definitions

As used throughout this application, including the claims, the following terms have the meanings defined below, unless specifically indicated otherwise. The phrases “compounds of Structure I”, “compound of the invention”, and “compound” are used interchangeably throughout the application and should be treated as synonyms.

The phrase “pharmaceutically acceptable” indicates that the designated carrier, vehicle, diluent, excipient, solvate, salt or prodrug is generally chemically and/or physically compatible with the other ingredients comprising a formulation, and is physiologically compatible with the recipient thereof.

The terms “treat(s)”, “treating”, “treated”, and “treatment” as used herein include preventative (e.g., prophylactic), ameliorative, palliative and curative uses and/or results. The terms preventative or prophylactic are used interchangeably and refer to treatment prior to the onset of one or more signs or symptoms of a particular condition or disease state. More specifically, these terms refer to the treatment of patients that are largely asymptomatic, i.e. where symptoms of a particular condition or disease state are not readily apparent or detectable, and which results in the substantial prevention, suppression or delay in the onset of one or more signs or symptoms of a particular condition or disease state. An ameliorative treatment is one that improves and/or lessens the severity of one or more signs or symptoms of a particular condition or disease state.

The phrases “therapeutic” and “therapeutically effective amount” as used herein respectively denote an effect and an amount of a compound, composition or medicament that (a) treats a particular disease, condition or disorder; (b) attenuates, ameliorates or eliminates one or more signs, symptoms of or complications arising from a particular disease, condition or disorder; (c) prevents or delays the onset of one or more signs, symptoms of or complications associated with a particular disease, condition or disorder. It should be understood that the terms “therapeutic” and “therapeutically effective amount” encompass any one of the aforementioned effects (a)-(c), either alone or in combination with any of the others (a)-(c). The terms “mammal”, “patient” and “subject” refer to warm blooded animals such as, for example, guinea pigs, mice, rats, gerbils, cats, rabbits, dogs, monkeys, chimpanzees, and humans. The “therapeutically effective amount” will vary depending on the composition, the compound, the therapy, the course of treatment, the disease, disorder, or condition, and its severity and the age, weight, etc., of the subject to be treated.

As used herein, the term “sebaceous glands” refers to microscopic glands in the skin that secrete an oily/waxy matter, called sebum, to lubricate and waterproof the skin and hair of mammals. In humans, they are found in greatest abundance on the face and scalp, though they are distributed throughout all skin sites except the palms and soles. In the eyelids, meibomian sebaceous glands secrete a special type of sebum into tears.

As used herein, the term “skin” refers to the outer covering of the body. In humans, it is the largest organ of the integumentary system. The skin has multiple layers of ectodermal tissue and guards the underlying muscles, bones, ligaments and internal organs. Human skin is similar to that of most other mammals, except that it is not protected by a fur. Though nearly all human skin is covered with hair follicles, it can appear hairless. There are two general types of skin, hairy and glabrous skin. The adjective cutaneous means “of the skin” (from Latin cutis, skin).

As used herein, the term “acne” refers to acne vulgaris, a common human skin disease, characterized by areas of skin with comedones (blackheads and whiteheads), papules (pinheads), nodules (large papules), pimples, and possibly scarring. Acne affects mostly skin with the densest population of sebaceous follicles; these areas include the face, the upper part of the chest, and the back. Severe acne is inflammatory, but acne can also manifest in non-inflammatory forms. Severe acne also includes the condition known as ‘nodulocystic acne’. Acne lesions are caused by changes in pilosebaceous units, skin structures consisting of a hair follicle and its associated sebaceous gland, changes that require androgen stimulation.

The term “seborrheic dermatitis” refers to a chronic disorder characterized by greasy or flaky scales overlying erythematous patches or plaques. The disorder is commonly located on areas of the skin in which sebaceous glands are located, including among other areas the scalp, face, auditory canal, and postauricular areas. The disorder may manifest itself in the first few weeks of life of humans, resolving before adolescence, but may also occur in adult life. It is typically treated with short-term therapies of low-potency steroids or topical anti-fungal agents such as ketoconazole cream or ciclopirox cream.

The term “rosacea” refers to a condition of reddening of the skin that occurs in the cheeks, nose, forehead, and chin. Patients with rosacea present with erythematous areas, telangiectases, papules, and/or pustules. The condition does not involve comedone formation, in distinction from acne, but may involve a vascular hyper-reactivity in the skin of the affected areas, and it may be accompanied by sebaceous overgrowth, especially on the nose. Previously, ‘rosacea’ has been referred to as ‘acne rosacea’.

As used herein, the term “adipocyte” refers to cells, also known as lipocytes and fat cells, which are the cells that primarily compose adipose tissue, specialized in storing energy as fat. There are two principal types of adipose tissue, white adipose tissue (WAT) and brown adipose tissue (BAT), which are also known as white fat and brown fat, respectively, and comprise two types of fat cells. WAT is the predominant type. In addition, approximately 10% of fat cells are renewed annually at all adult ages and levels of body mass index (Spalding (2008)). Most recently, the presence of beige adipocytes with a gene expression pattern distinct from either white or brown adipocytes has been described. Also another special type of adipose tissue is being studied, pink adipose tissue, which seems to be involved in mammillary duct development in female breasts. Regulation of some of the same biochemical pathways as in the sebaceous glands can also occur in adipose tissue, so yet other applications involve the potential diminution and/or removal of fat cells in conditions such as excess submental fat or excess fat in other body areas, and body sculpting.

As used herein, “lipomas” refer to a common benign tumor involving the proliferation of fat cells (adipocytes). “Liposarcomas” refer to a highly malignant and aggressive cancer of adipocytes.

As used herein, the term “keratinocyte” refers to the predominant cell type in the epidermis, the outermost layer of the skin, constituting 90% of the cells found there. Those keratinocytes found in the basal layer (stratum basale) of the skin are sometimes referred to as “basal cells” or “basal keratinocytes”.

As used herein, the term “hepatocyte” refers to a cell of the main tissue of the liver. Hepatocytes make up 70-85% of the liver's cytoplasmic mass. These cells are involved in protein synthesis, protein storage, transformation of carbohydrates, synthesis of cholesterol, bile salts and phospholipids, detoxification, modification, and excretion of exogenous and endogenous substances. The hepatocyte also initiates formation and secretion of bile.

As used herein the term “sebocyte” refers to epithelial cells that originate from a basal cell layer at the periphery of the sebaceous gland. Differentiation and maturation of sebocytes is accompanied by the accumulation of increasing amounts of a unique mixture of lipids (sebum). Approximately 25% of human sebaceous lipids are wax esters that are not synthesized by other cells in the body. With respect to lipogenesis, sebocyte differentiation may follow a similar program of differentiation as that observed in adipocytes. These lipid-laden cells then migrate towards the central excretory duct. Eventually, the cells disintegrate and release their lipid content. Most of the lipids of the skin surface come from sebaceous gland secretions.

As used herein, the term “lipid modulator” refers to any molecule, compound or composition that is capable of either modulating the secretion of triacyl glycerides/waxes/fatty acids or modulating the synthesis of multiple enzymes involved in lipid metabolism.

As used herein, the term “EROD” refers to the ethoxyresorufin-O-deethylase (EROD) assay which monitors the induction of the xenobiotic-metabolizing enzyme cytochrome P-450 1A1 (CYP1A1)

As used herein, the term “CALUX” refers to Chemical-Activated Luciferase Gene Expression (CALUX), which can also be used for measuring activation of Cyp1A1.

As used herein, the term “CYP1A1” refers to Cytochrome P450, family 1, subfamily A, polypeptide 1. CYP1A1 is a protein that in humans is encoded by the CYP1A1 gene. The protein is a member of the cytochrome P450 superfamily of enzymes. CYP1A1 is involved in phase I xenobiotic and drug metabolism and is also known as AHH (aryl hydrocarbon hydroxylase).

As used herein, the term “lipophilicity” refers to the ability of a chemical compound to dissolve in fats, oils, lipids, and non-polar solvents such as hexane or toluene. These non-polar solvents are themselves lipophilic (translated as “fat-loving” or “fat-liking”) with the axiom that like dissolves like generally holding true. Thus lipophilic substances tend to dissolve in other lipophilic substances, while hydrophilic (water-loving) substances tend to dissolve in water and other hydrophilic substances. Lipophilicity, hydrophobicity, and non-polarity can describe the same tendency towards participation in the London dispersion force as the terms are often used interchangeably. However, the terms “lipophilic” and “hydrophobic” are not synonymous, as can be seen with silicones and fluorocarbons, which are hydrophobic but not lipophilic

“Alkyl” means a straight or branched chain, saturated hydrocarbon radical. By way of example, the hydrocarbon chain may have from one to twenty carbons, one to sixteen carbons, one to fourteen carbons, one to twelve carbons, one to ten carbons, one to eight carbons, one to six carbons, one to four carbons, etc. “Lower alkyl” may refer to alkyls having, e.g., one to six carbons, one to four carbons, etc. In certain examples, a straight chain alkyl may have from one to six carbon atoms and a branched alkyl three to six carbon atoms, e.g., methyl, ethyl, propyl, 2-propyl, butyl (including all isomeric forms), pentyl (including all isomeric forms), and the like. “Me” means methyl, “Et” means ethyl, and “iPr” means isopropyl. Alkyl may be optionally substituted, e.g., optionally substituted with oxygen, silicon, sulphur or optionally substituted with OH, O-alkyl, SH, S-alkyl, NH₂, NH-alkyl. In another example, alkyl may be C₁ to C₁₂ straight chain or branched chain alkyl optionally substituted with oxygen, silicon, sulphur or optionally substituted with OH, O-alkyl, SH, S-alkyl, NH₂, NH-alkyl.

“Alkylene” means a divalent alkyl, with alkyl as defined above.

“Aryl” means a monocyclic or bicyclic aromatic hydrocarbon radical, e.g., having from of 6 to 20 or 6 to 10 ring atoms e.g., phenyl or naphthyl. Aryl may be optionally substituted, e.g., substituted phenyl or substituted naphthyl.

“Alkylaryl” means a (alkylene)—R radical where R is aryl as defined above. Alkylaryl may be optionally substituted. In certain examples, alkylaryl may be alkylphenyl, alkylsubstituted phenyl, alkylnaphthyl or alkylsubstituted naphthyl.

“Alkenyl” means a straight or branched chain, saturated hydrocarbon radical which contains a carbon-carbon double bond. By way of example, the hydrocarbon chain may have from two to twenty carbons, two to sixteen carbons, two to fourteen carbons, two to twelve carbons, two to ten carbons, two to eight carbons, two to six carbons, two to four carbons, etc. “Lower alkenyl” may refer to alkenyls having, e.g., two to six carbons, two to four carbons, etc. In certain examples, a straight chain alkenyl may have from two to six carbon atoms and a branched alkyl three to six carbon atoms, e.g., a vinyl group, an allyl group, butene (including all isomeric forms), pentane (including all isomeric forms), and the like. Alkenyl may be optionally substituted. In certain examples, alkenyl may be a C₁ to Cₐ straight chain or branched chain hydrocarbon containing a carbon-carbon double bond, optionally substituted with oxygen, silicon or sulphur or optionally substituted with OH, O-alkyl, SH, S-alkyl, NH₂ or NH-alkyl.

“Alkynyl” means a straight or branched chain, saturated hydrocarbon radical which contains a carbon-carbon triple bond. By way of example, the hydrocarbon chain may have from two to twenty carbons, two to sixteen carbons, two to fourteen carbons, two to twelve carbons, two to ten carbons, two to eight carbons, two to six carbons, two to four carbons, etc. “Lower alkynyl” may refer to alkynyls having, e.g., two to six carbons, two to four carbons, etc. in certain examples, a straight chain alkynyl may have from two to six carbon atoms and a branched alkyl three to six carbon atoms, e.g., an acetylene group, a propargyl group, butyne (including all isomeric forms), pentyne (including all isomeric forms), and the like. Alkynyl may be optionally substituted. In certain examples, alkynyl may be a C₁ to C₁₂ straight chain or branched chain hydrocarbon containing a carbon-carbon triple bond, optionally substituted with oxygen, silicon or sulphur or optionally substituted with OH, O-alkyl, SH, S-alkyl, NH₂ or NH-alkyl.

“Cycloalkyl” means a cyclic saturated or partially saturated hydrocarbon radical (or an alicyclic radical). By way of example, the cycloalkyl may have from three to twenty carbon atoms, from three to sixteen carbon atoms, from three to fourteen carbon atoms, from three to twelve carbon atoms, from three to ten carbon atoms, from three to eight carbon atoms, from three to seven carbon atoms, from three to six carbon atoms, etc., wherein one or two carbon atoms may be replaced by an oxo group, e.g., admantanyl, cyclopropyl, cyclobutyl, cyclopentyl, cyclohexyl, cyclohexenyl, indanyl and the like.

“Alkylcycloalkyl” means a (alkylene)—R radical where R is cycloalkyl as defined above; e.g., cyclopropylmethyl, cyclobutylmethyl, cyclopentylethyl, or cyclohexylmethyl, and the like. In another example, alkylcycloalkyl has four to twelve carbon atoms, i.e., Cₐ-Cᵤ alkylcycloalkyl.

“O-alkyl” means an (oxygen)—R radical where R is alkyl as defined above. For example, O-alkyl may be an oxygen atom bonded to a C₁ to C₆ straight chain or branched chain alkyl.

“O-cycloalkyl” means an (oxygen)-R radical where R is cycloalkyl as defined above. For example, O-cycloalkyl is an oxygen atom bonded to a C₃ to C₇ cycloalkyl.

“O-alkylcycloalkyl” means an (oxygen)—R radical where R is alkylcycloalkyl as defined above. For example, O-cycloalkyl is an oxygen atom bonded to a C4 to C₈ alkylcycloalkyl.

“Heterocyclyl” or “heterocycloalkyl” means a saturated or unsaturated monocyclic group, in which one or two ring atoms are heteroatom selected from N, O, or S, the remaining ring atoms being C. Heterocyclyl and heterocycloalkyl includes, e.g., where the heterocycle comprises one or two hetero atoms selected from O, S, or N, including a C₂ to C₆ heterocycloalkyl. The heterocyclyl ring is optionally fused to a tone) aryl or heteroaryl ring as defined herein. The heterocyclyl ring fused to monocyclic aryl or heteroaryl ring is also referred to in this Application as “bicyclic heterocyclyl” ring. Additionally, one or two ring carbon atoms in the heterocyclyl ring can optionally be replaced by a —CO-group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring is unsaturated it can contain one or two ring double bonds. When the heterocyclyl group contains at least one nitrogen atom, it is also referred to herein as heterocycloamino and is a subset of the heterocyclyl group. When the heterocyclyl group is a saturated ring and is not fused to aryl or heteroaryl ring as stated above, it is also referred to herein as saturated monocyclic heterocyclyl.

“Alkylheterocycloalkyl” means an -(alkylene)-R radical where R is hetero cyclyl ring as defined above e.g., tetraydrofuranylmethyl, piperazinylmethyl, morpholinylethyl, and the like. Alkylheterocycloalkyl also includes, e.g., where the heterocycle comprises one or two hetero atoms selected from O, S, or N and has three to eleven carbon atoms, i.e., C₃ to C₁₁ alkylheterocycloalkyl, and includes when N is present in the heterocyclic ring the nitrogen atom may be in the form of an amide, carbamate or urea.

“Heteroaryl” means a monocyclic or bicyclic aromatic radical, where one or more, preferably one, two, or three, ring atoms are heteroatom selected from N, O, or S, the remaining ring atoms being carbon. Representative examples include, but are not limited to, pyrrolyl, thienyl (thiophenyl), thiazolyl, imidazolyl, furanyl, indolyl, isoindolyl, oxazolyl, isoxazolyl, diazolyl, pyrazolyl, triazolyl, benzothiazolyl, benzoxazolyl, quinolinyl, isoquinolinyl, pyridinyl (pyridyl), pyrimidinyl, pyrazinyl, pyridazinyl, tetrazolyl, and the like. Heteroaryl may be optionally substituted.

“Oxo” or “carbonyl” means a ═(O) group or C═O group, respectively.

The term “substituted” means that the referenced group is substituted with one or more additional group(s) individually and independently selected from groups described herein. In some embodiments, an optional substituent is selected from oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoroalkyl, substituted or unsubstituted heteroalkyl, substituted or unsubstituted alkoxy, fluoroalkoxy, —S-alkyl, —S(O)₂-alkyl, —CONH((substituted or unsubstituted alkyl) or (substituted or unsubstituted phenyl)), —CON(H or alkyl)₂, —OCON(substituted or unsubstituted alkyl)₂, —NHCONH((substituted or unsubstituted alkyl) or (substituted or unsubstituted phenyl)), —NHCOalkyl, —N(substituted or unsubstituted alkyl)CO(substituted or unsubstituted alkyl), —NHCOO(substituted or unsubstituted alkyl), —C(OH)(substituted or unsubstituted alkyl)₂, and —C(NH2)(substituted or unsubstituted alkyl)₂. In some embodiments, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(O)₂—CH₃, —CONH₂, —CONHCH₃, —NHCONHCH₃, —COCH₃, —COON and the like. In some embodiments, substituted groups are substituted with one, two or three of the preceding groups. In some embodiments, substituted groups are substituted with one or two of the preceding groups. In some embodiments, substituted groups are substituted with one of the preceding groups. Further, unless stated to the contrary, a formula with chemical bonds shown only as solid lines and not as wedges or dashed lines contemplates each possible isomer, e.g., each enantiomer and diastereomer, and a mixture of isomers, such as racemic or scalemic mixtures.

When the compounds described herein include one or more chiral centers, the stereochemistry of such chiral centers can independently be in the R or S configuration, or a mixture of the two. The chiral centers can be further designated as R or S or R,S or d,D, I,L or d,I, D,L. Correspondingly, the compounds of the invention, if they can be present in optically active form, can actually be present in the form of a racemic mixture of enantiomers, or in the form of either of the separate enantiomers in substantially isolated and purified form, or as a mixture comprising any relative proportions of the enantiomers.

When the compounds described herein contain two or more chiral centers then diastereomers are possible. Such diastereomers may be present as pure diastereometric enantiomers, pure racemic mixtures of diastereomeric enantiomers, or mixtures of diastereomers which may be racemic or may have optical activity in their own right due to complex permutations of enantiomeric diastereomers in the balance of the mixtures.

When the compounds of the invention, if they can be present in geometrically isomeric forms around, for example, a substituent bond, then they can actually be present in the form of a mixture of geometric isomers comprising any relative proportions of the isomers, or in some cases in the form of either of the separate geometric isomers in substantially isolated and purified form.

When the compounds described herein include one or more isolated or linearly conjugated double bonds, the geometry around such double bonds can be independently a cis/trans, E/Z mixture or an E or Z geometric isomer thereof.

The compounds of the present invention may exist in unsolvated as well as a variety of solvated forms with pharmaceutically acceptable solvents such as water, ethanol, and the like. In general, the solvated forms are considered equivalent to the unsolvated forms for the purposes of the present invention. It should be understood that pharmaceutically acceptable solvents further includes isotopically substituted solvents such as D₂O, dimethyl sulfoxide-d6 and the like. The term ‘solvate’ is used herein to describe a complex comprising the compound of the invention and one or more pharmaceutically acceptable solvent molecules, including water. As such, all manner of hydrates of the compound are included by the term ‘solvate’. It is intended that the present invention embrace unsolvated forms, solvated forms and mixtures of solvated forms in any ratio.

The compound of the present invention and/or its salts and/or solvate may exist as amorphous solids or may exist in one or more crystalline states, i.e. polymorphs. Polymorphs of the compound of Structure I are encompassed in the present invention and may be prepared by crystallization under a number of different conditions such as, for example, using different solvents or different solvent mixtures; crystallization at different temperatures; and using various modes of cooling ranging from very fast to very slow during crystallization. Polymorphs may also be obtained by heating or melting a compound of

Structure I followed by gradual or fast cooling. The presence of polymorphs may be determined by solid NMR spectroscopy, IR spectroscopy, differential scanning calorimetry, powder x-ray diffraction or other techniques. It should be understood that all such crystalline and amorphous forms of the compound of Structure I, and its salts, solvates and prodrugs thereof are encompassed by the invention and the claims.

The present invention also includes all pharmaceutically acceptable isotopically-labeled variations of the compound of Structure I. Such isotopically-labeled variations are compounds having the same structure and molecular formula as the compound of Structure I but wherein one or more atoms are replaced by atoms having an atomic mass or mass number different from the atomic mass or mass number usually found in nature. Examples of isotopes that may be incorporated into the compound of the present invention include isotopes of hydrogen, carbon, fluorine, nitrogen, and oxygen, such as ²H, ³H, ¹¹C, ¹³C, ¹⁴C, ¹⁸F, ¹³N ¹⁵N ¹⁷O and ¹⁸O, respectively.

Certain isotopically labeled variations of the compound of the present invention such as, for example, those incorporating a radioactive isotope such as ³H and ¹⁴C, are useful in drug and/or substrate tissue distribution studies. Tritium, i.e. ³H, and carbon-14, i.e. ¹⁴C, are particularly preferred due their ease of preparation and detection. Further, substitution with heavier isotopes such as deuterium, i.e. ²H, can afford certain therapeutic advantages resulting from greater metabolic stability, for example, increased in vivo half-life or reduced dosage requirements, and hence may be preferred in some circumstances, Isotopically labeled compounds of Structure I of this invention and prodrugs thereof can generally be prepared by carrying out the procedures disclosed in the Schemes and/or in the Examples by substituting a readily available isotopically labeled reagent for a non-isotopically labeled reagent.

The compounds of Structure I may be administered as a prodrug. The term prodrug refers to a compound which is transformed in viva to a compound of Structure I, or a pharmaceutically acceptable salt or solvate of the compound. The transformation may occur by various mechanisms, such as via hydrolysis in blood. A prodrug of the compound of Structure I may be formed in a conventional manner according to methods known in the art. A thorough discussion of prodrugs Is provided by V. Stella in Pro-drugs as Novel Delivery Systems, Vol. 14 of the A.C.S. Symposium Series (Stella (1975)), and in Bioreversible Carriers in Drug Design (Roche (1987)), both of which are incorporated herein by reference.

In some embodiments, a compound of the disclosure is present in a composition as a salt. In some embodiments, salts are obtained by reacting a compound of the disclosure with acids. In some other embodiments, pharmaceutically acceptable salts are obtained by reacting a compound of the disclosure with a base. In other embodiments, the compounds are used as free-acid or free-base form in the manufacture of the compositions described herein. The type of salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3- (4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-(2.2.2loct-2-ene-l-carboxylic acid, giucoheptonic acid, 4,4′-methylenebis-(3-hydroxy-2-ene-l-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, the lipid modulating compound described herein are reacted with an organic base, such as, but not limited to, ethanolamine, diethanolamine, triethanolamine, methylamine, dimethylamine, trimethylamine, ethylamine, diethylamine, triethylamine, N-methylglucamine, dicyclohexylamine, tris(hydroxymethyl)methylamine. In other cases, the compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like. In one specific embodiment, the compound of Structure I is prepared as hydrochloride, hydrobromide, acetate, propionate, butyrate, sulphate, hydrogen sulphate, sulphite, carbonate, hydrogen carbonate, phosphate, phosphinate, oxalate, hemi-oxalate, malonate, hemi-malonate, fumarate, hemi-fumarate, maleate, hemi-maleate, citrate, hemi-citrate, tartrate, hemi-tartrate, aspartate, or glutamate salt.

In one embodiment, the compounds of the disclosure may be prepared as a three component salt form including the components A, B, and C wherein:

A is the protonated form of a natural or unnatural amino acid;

B is the dianion of an acid; and

C is the protonated form of a Compound of Structure I.

In certain aspects of the above embodiment, stoichiometric amounts of A, B, and C may be included wherein: A is the protonated form of a natural amino acid selected from alanine, aspartic acid, asparagine, arginine, glycine, glutamine, glutamic acid lysine, phenylalanine, tyrosine, serine, threonine, tryptophan, leucine, isoleucine, histidine, methionine, proline, cysteine, or cystine; B is the dianion of an acid selected from oxalic, malonic, citric, maleic, fumaric, tartaric, aspartic, glutamic acids and the like; and C is the protonated form of a compound of structure I.

In the scope of the embodiments, the compounds described herein include further forms of the compounds such as pharmaceutically acceptable salts, solvates (including hydrates), amorphous phases, partially crystalline and crystalline forms (including all polymorphs), prodrugs, metabolites, N-oxides, isotopically-labeled, epimers, pure epimers, epimer mixtures, enantiomers Including but not limited to single enantiomers and enantiomeric diastereomers, meso compounds, stereoisomers, racemic mixtures and diasteroisomeric mixtures. Compounds described herein having one or more double bonds include cis/trans isomers, E/Z isomers and geometric isomers.

In some embodiments, sites on the compounds disclosed herein are susceptible to various metabolic reactions. Therefore incorporation of appropriate substituents at the places of metabolic reactions will reduce, minimize or eliminate the metabolic pathways. In specific embodiments, the appropriate substituent to decrease or eliminate the susceptibility of the aromatic ring to metabolic reactions is, by way of example only, a halogen, deuterium or an alkyl group. Examples of such substituents can be found in Burger's Medicinal Chemistry, Drug Discovery and Development, 8 Volume Set (Abraham (2010)) and in Foye's Principles of Medicinal Chemistry (Lemke (2012)).

In some embodiments, sites on the compounds disclosed herein are not susceptible to various metabolic reactions. Therefore incorporation of appropriate substituents at or near or distant from the places of a lack of metabolic reactions will modulate, enhance, or maximize the metabolic pathways. In specific embodiments, the appropriate substituent (metabolic handle) to enhance, or maximize the susceptibility of the aromatic ring to metabolic reactions is, by way of example only, is a phenolic or methoxy or carboxylate group. Examples of such substituents can be found in Burger's Medicinal Chemistry, Drug Discovery and Development, 8 Volume Set (Abraham (2010)) and in Foye's Principles of Medicinal Chemistry (Lemke (2012)).

Throughout the specification, groups and substituents thereof can be chosen by one skilled in the field to provide stable moieties and compounds.

Synthesis of the Compounds

In general, compounds of Structure I may be prepared using a number of methods known in the chemical arts, particularly in light of the description contained herein, in combination with the knowledge of the skilled artisan. Various starting materials, intermediates, and reagents may be purchased from commercial sources or made according to literature methods or adaptations thereof. Although other reagents, compounds or methods can be used in practice or testing, generalized methods for the preparation of the compound of Structure I are illustrated by the following descriptions and reaction Schemes. The methods disclosed herein, including those outlined in the Schemes, descriptions, and Examples are for intended for illustrative purposes and are not to be construed in any manner as limitations thereon. Various changes and modifications will be obvious to those of skill in the art given the benefit of the present disclosure and are deemed to be within the spirit and scope of the present disclosure as further defined in the appended claims.

Although specific embodiments of various aspects of the invention will be described with reference to the Schemes, Preparations and/or Examples, it should be understood that such embodiments are by way of example only and are merely illustrative of a small number of the many possible specific embodiments which can represent applications of the principles of the present disclosure. The starting materials used for the synthesis of compounds described herein can be obtained from commercial sources, such as Aldrich Chemical Co. (Milwaukee, Wis.), Sigma Chemical Co. (St. Louis, Mo.), or the starting materials can be synthesized. The compounds described herein, and other related compounds having different substituents can be synthesized using techniques and materials known to those of skill in the art, such as described, for example, in March's Advanced Organic Chemistry: Reactions, Mechanisms, and Structure (Smith (2013)), Design and Strategy in Organic Synthesis (Hanessian (2013)) Greene's Protective Groups in Organic Synthesis (Wets (2006)) and Fiesers' Reagents for Organic Synthesis (Volumes 1- 27) (Ho (2013)), each of which are incorporated by reference in their entirety.

General methods for the preparation of the compounds as disclosed herein may be derived from known reactions in the field, and the reactions may be modified by the use of appropriate reagents and conditions, as would be recognized by the skilled person, for the introduction of the various moieties found in the formulae as provided herein.

The intermediate products described can be recovered by extraction, evaporation, or other techniques known in the art. The crude materials may then be optionally purified by chromatography, H PLC, recrystallization, trituration, distillation, or other techniques known in the art. In the discussions below, the following abbreviations were used: EtOH (ethanol) NaOH (sodium ethoxide), DMSO (dimethylsulfoxide), MOM (methoxymethyl), THF (tetrahydrofuran), Dess-Martin (Dess-Martin Periodinane) and TBS (tert-butyldimethylsilyl).

As would be appreciated by those skilled in the art, some of the methods useful for the preparation of such compounds, as discussed above, may require protection of a particular functionality, e.g., to prevent interference by such functionality in reactions at other sites within the molecule or to preserve the Integrity of such functionality. The need for, and type of, such protection is readily determined by one skilled in the art, and will vary depending on, for example, the nature of the functionality and the conditions of the selected preparation method. Methods of introducing and removing protecting groups are well known to those of ordinary skill in the art and are described in Greene's Protective Groups in Organic Synthesis (Wuts (2006)). Alternate reagents, starting materials, as well as methods for optimizing or adapting the procedures described herein would also be readily determined by one skilled in the art.

The Suzuki reaction (Palladium catalyzed coupling of an Aryl halide or triflate with a boronic acid derivative) and variants thereof can also be used in a variety of ways for the synthesis of this class of compounds. Scheme 1 below shows the synthesis of a key intermediate prior to iodination.

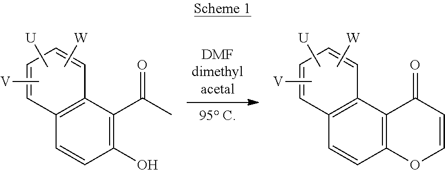

Subsequent iodination in preparation for the Suzuki reaction can be achieved in a two-step procedure involving initial ring opening with piperidine as shown in Scheme 2.

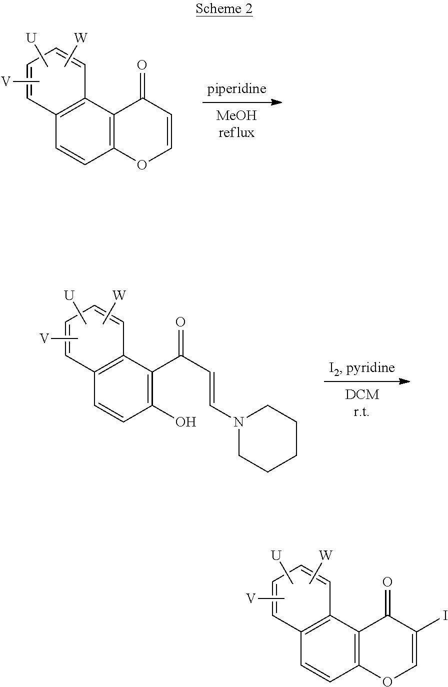

The Suzuki reaction between the iodinated intermediate from above and a suitable aromatic boronic acid derivative gives the final compounds as shown in Scheme 3.

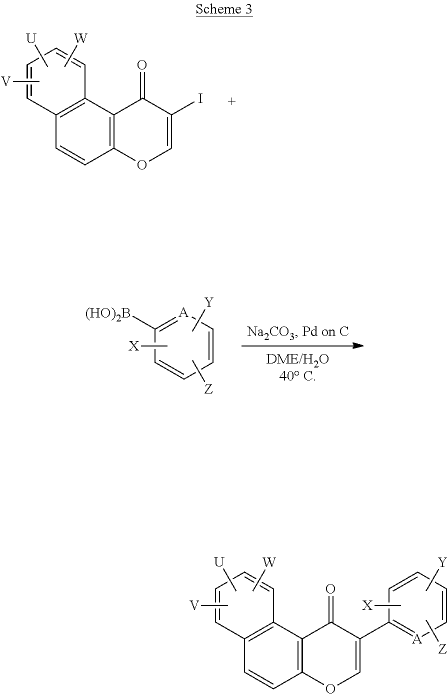

Other variations on the Suzuki reaction are also envisaged for the synthesis of this class of compounds (Selepe (2013)).

Other methods of synthesis are also envisaged including, but not limited, to variations of methods described in the following publications: (Adib (2008); Bianco (2003); Bohm (1998); Cushman (1991); Fujita (2010); Juvale (2013); Kulkarni (2012); Liu (2013)).

The modular syntheses of Schemes 1 through 3 can all be adapted to automated synthesis platforms, focused library platforms, solid phase organic synthesis platforms, combinatorial chemistry platforms, microwave chemistry platforms and other modern variants of synthetic organic chemistry suitable for high throughput.

Tables 1, 2, 3 and 4 list the specific compounds synthesized via the overall syntheses and general methods outlined in this section

TABLE 1
Structure

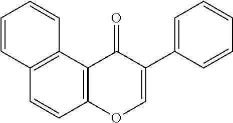 [SMILES: O=c1c(-c2ccccc2)coc2ccc3ccccc3c12]

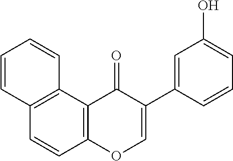 [SMILES: O=c1c(-c2cccc(O)c2)coc2ccc3ccccc3c12]

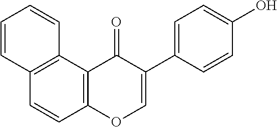 [SMILES: O=c1c(-c2ccc(O)cc2)coc2ccc3ccccc3c12]

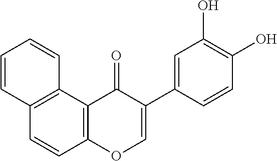 [SMILES: O=c1c(-c2ccc(O)c(O)c2)coc2ccc3ccccc3c12]

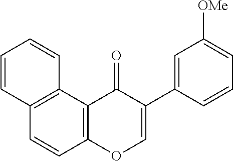 [SMILES: COc1cccc(-c2coc3ccc4ccccc4c3c2=O)c1]

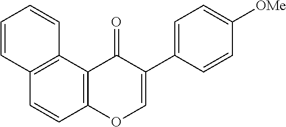 [SMILES: COc1ccc(-c2coc3ccc4ccccc4c3c2=O)cc1]

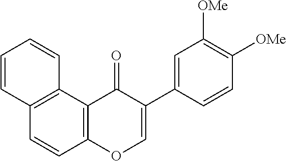 [SMILES: COc1ccc(-c2coc3ccc4ccccc4c3c2=O)cc1OC]

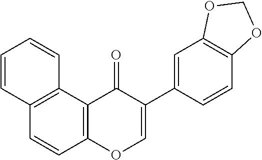 [SMILES: O=c1c(-c2ccc3c(c2)OCO3)coc2ccc3ccccc3c12]

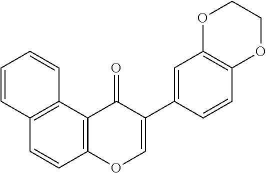 [SMILES: O=c1c(-c2ccc3c(c2)OCCO3)coc2ccc3ccccc3c12]

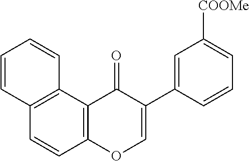 [SMILES: COC(=O)c1cccc(-c2coc3ccc4ccccc4c3c2=O)c1]

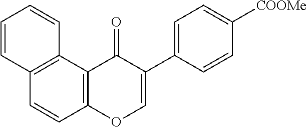 [SMILES: COC(=O)c1ccc(-c2coc3ccc4ccccc4c3c2=O)cc1]

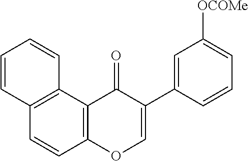

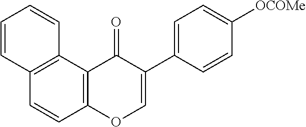

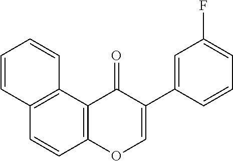 [SMILES: O=c1c(-c2cccc(F)c2)coc2ccc3ccccc3c12]

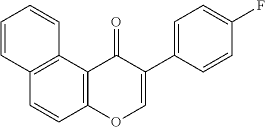 [SMILES: O=c1c(-c2ccc(F)cc2)coc2ccc3ccccc3c12]

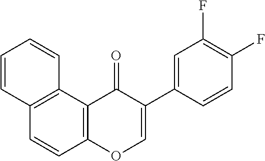 [SMILES: O=c1c(-c2ccc(F)c(F)c2)coc2ccc3ccccc3c12]

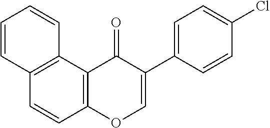 [SMILES: O=c1c(-c2ccc(Cl)cc2)coc2ccc3ccccc3c12]

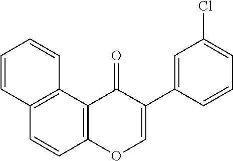 [SMILES: O=c1c(-c2cccc(Cl)c2)coc2ccc3ccccc3c12]

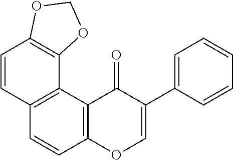 [SMILES: O=c1c(-c2ccccc2)coc2ccc3ccc4c(c3c12)OCO4]

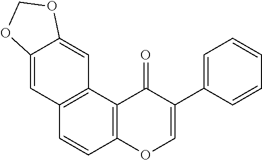 [SMILES: O=c1c(-c2ccccc2)coc2ccc3cc4c(cc3c12)OCO4]

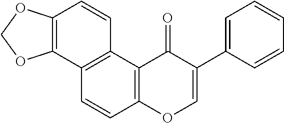 [SMILES: O=c1c(-c2ccccc2)coc2ccc3c4c(ccc3c12)OCO4]

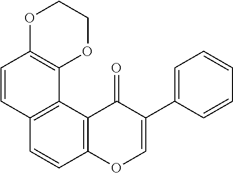 [SMILES: O=c1c(-c2ccccc2)coc2ccc3ccc4c(c3c12)OCCO4]

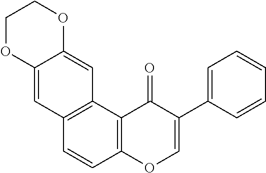 [SMILES: O=c1c(-c2ccccc2)coc2ccc3cc4c(cc3c12)OCCO4]

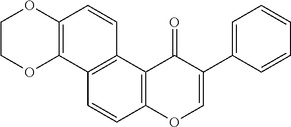 [SMILES: O=c1c(-c2ccccc2)coc2ccc3c4c(ccc3c12)OCCO4]

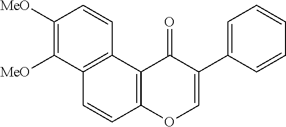 [SMILES: COc1ccc2c(ccc3occ(-c4ccccc4)c(=O)c32)c1OC]

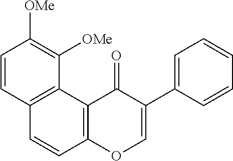 [SMILES: COc1ccc2ccc3occ(-c4ccccc4)c(=O)c3c2c1OC]

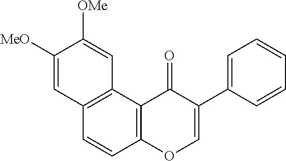 [SMILES: COc1cc2ccc3occ(-c4ccccc4)c(=O)c3c2cc1OC]

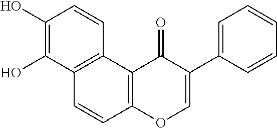 [SMILES: O=c1c(-c2ccccc2)coc2ccc3c(O)c(O)ccc3c12]

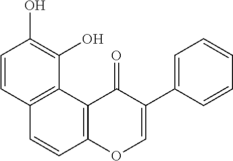 [SMILES: O=c1c(-c2ccccc2)coc2ccc3ccc(O)c(O)c3c12]

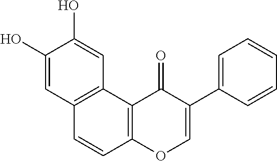 [SMILES: O=c1c(-c2ccccc2)coc2ccc3cc(O)c(O)cc3c12]

TABLE 2
Structure

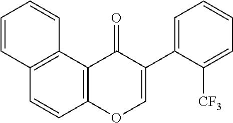 [SMILES: O=c1c(-c2ccccc2C(F)(F)F)coc2ccc3ccccc3c12]

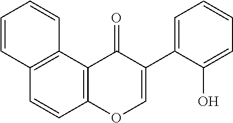 [SMILES: O=c1c(-c2ccccc2O)coc2ccc3ccccc3c12]

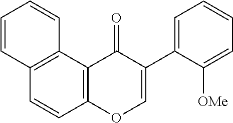 [SMILES: COc1ccccc1-c1coc2ccc3ccccc3c2c1=O]

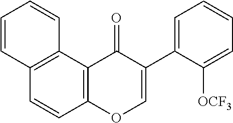 [SMILES: O=c1c(-c2ccccc2OC(F)(F)F)coc2ccc3ccccc3c12]

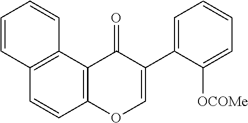

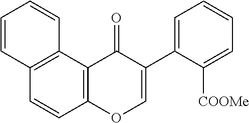 [SMILES: COC(=O)c1ccccc1-c1coc2ccc3ccccc3c2c1=O]

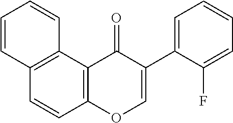 [SMILES: O=c1c(-c2ccccc2F)coc2ccc3ccccc3c12]

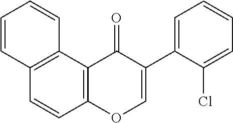 [SMILES: O=c1c(-c2ccccc2Cl)coc2ccc3ccccc3c12]

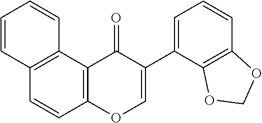 [SMILES: O=c1c(-c2cccc3c2OCO3)coc2ccc3ccccc3c12]

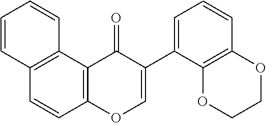 [SMILES: O=c1c(-c2cccc3c2OCCO3)coc2ccc3ccccc3c12]

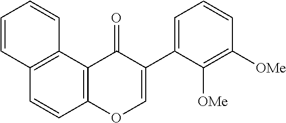 [SMILES: COc1cccc(-c2coc3ccc4ccccc4c3c2=O)c1OC]

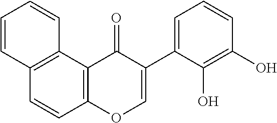 [SMILES: O=c1c(-c2cccc(O)c2O)coc2ccc3ccccc3c12]

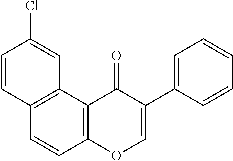 [SMILES: O=c1c(-c2ccccc2)coc2ccc3ccc(Cl)cc3c12]

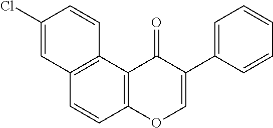 [SMILES: O=c1c(-c2ccccc2)coc2ccc3cc(Cl)ccc3c12]

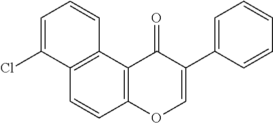 [SMILES: O=c1c(-c2ccccc2)coc2ccc3c(Cl)cccc3c12]

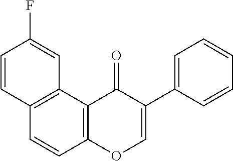 [SMILES: O=c1c(-c2ccccc2)coc2ccc3ccc(F)cc3c12]

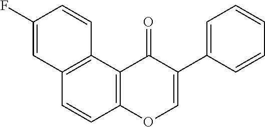 [SMILES: O=c1c(-c2ccccc2)coc2ccc3cc(F)ccc3c12]

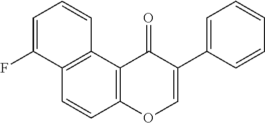 [SMILES: O=c1c(-c2ccccc2)coc2ccc3c(F)cccc3c12]

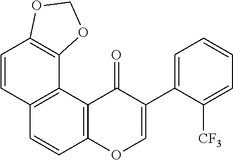 [SMILES: O=c1c(-c2ccccc2C(F)(F)F)coc2ccc3ccc4c(c3c12)OCO4]

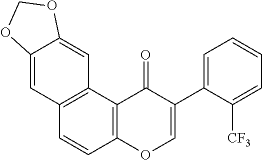 [SMILES: O=c1c(-c2ccccc2C(F)(F)F)coc2ccc3cc4c(cc3c12)OCO4]

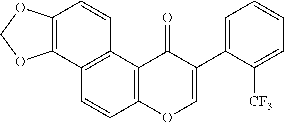 [SMILES: O=c1c(-c2ccccc2C(F)(F)F)coc2ccc3c4c(ccc3c12)OCO4]

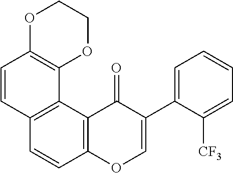 [SMILES: O=c1c(-c2ccccc2C(F)(F)F)coc2ccc3ccc4c(c3c12)OCCO4]

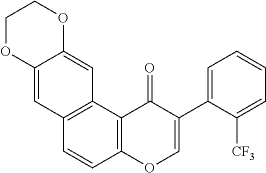 [SMILES: O=c1c(-c2ccccc2C(F)(F)F)coc2ccc3cc4c(cc3c12)OCCO4]

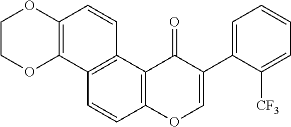 [SMILES: O=c1c(-c2ccccc2C(F)(F)F)coc2ccc3c4c(ccc3c12)OCCO4]

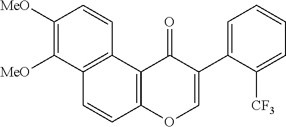 [SMILES: COc1ccc2c(ccc3occ(-c4ccccc4C(F)(F)F)c(=O)c32)c1OC]

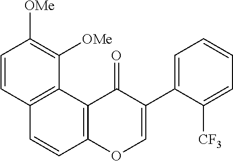 [SMILES: COc1ccc2ccc3occ(-c4ccccc4C(F)(F)F)c(=O)c3c2c1OC]

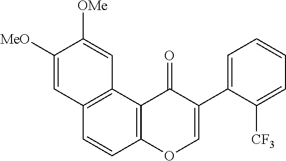 [SMILES: COc1cc2ccc3occ(-c4ccccc4C(F)(F)F)c(=O)c3c2cc1OC]

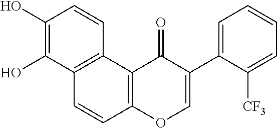 [SMILES: O=c1c(-c2ccccc2C(F)(F)F)coc2ccc3c(O)c(O)ccc3c12]

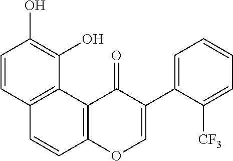 [SMILES: O=c1c(-c2ccccc2C(F)(F)F)coc2ccc3ccc(O)c(O)c3c12]

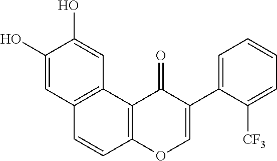 [SMILES: O=c1c(-c2ccccc2C(F)(F)F)coc2ccc3cc(O)c(O)cc3c12]

TABLE 3
Structure

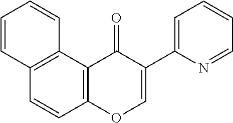 [SMILES: O=c1c(-c2ccccn2)coc2ccc3ccccc3c12]

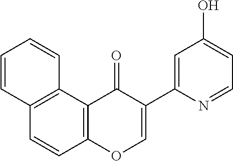 [SMILES: O=c1c(-c2cc(O)ccn2)coc2ccc3ccccc3c12]

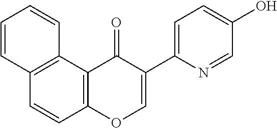 [SMILES: O=c1c(-c2ccc(O)cn2)coc2ccc3ccccc3c12]

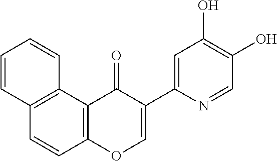 [SMILES: O=c1c(-c2cc(O)c(O)cn2)coc2ccc3ccccc3c12]

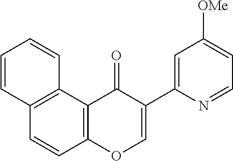 [SMILES: COc1ccnc(-c2coc3ccc4ccccc4c3c2=O)c1]

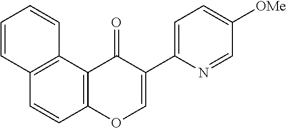 [SMILES: COc1ccc(-c2coc3ccc4ccccc4c3c2=O)nc1]

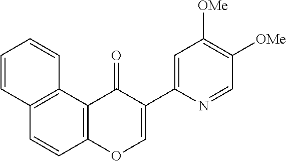 [SMILES: COc1cnc(-c2coc3ccc4ccccc4c3c2=O)cc1OC]

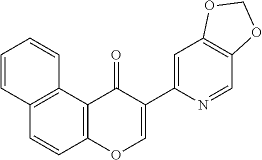 [SMILES: O=c1c(-c2cc3c(cn2)OCO3)coc2ccc3ccccc3c12]

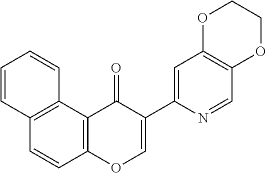 [SMILES: O=c1c(-c2cc3c(cn2)OCCO3)coc2ccc3ccccc3c12]

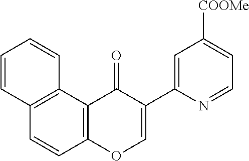 [SMILES: COC(=O)c1ccnc(-c2coc3ccc4ccccc4c3c2=O)c1]

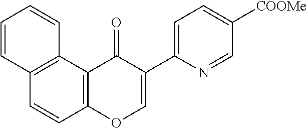 [SMILES: COC(=O)c1ccc(-c2coc3ccc4ccccc4c3c2=O)nc1]

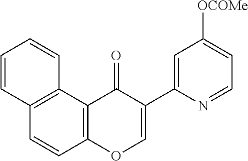

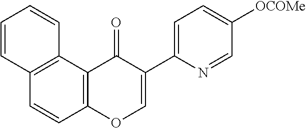

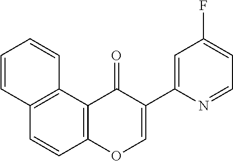 [SMILES: O=c1c(-c2cc(F)ccn2)coc2ccc3ccccc3c12]

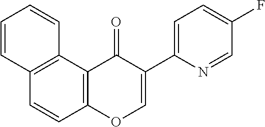 [SMILES: O=c1c(-c2ccc(F)cn2)coc2ccc3ccccc3c12]

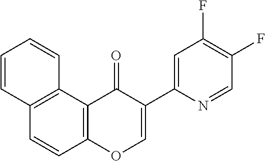 [SMILES: O=c1c(-c2cc(F)c(F)cn2)coc2ccc3ccccc3c12]

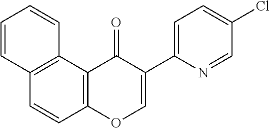 [SMILES: O=c1c(-c2ccc(Cl)cn2)coc2ccc3ccccc3c12]

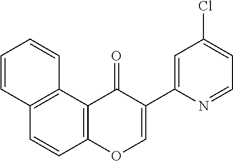 [SMILES: O=c1c(-c2cc(Cl)ccn2)coc2ccc3ccccc3c12]

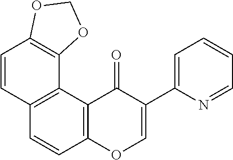 [SMILES: O=c1c(-c2ccccn2)coc2ccc3ccc4c(c3c12)OCO4]

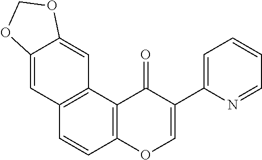 [SMILES: O=c1c(-c2ccccn2)coc2ccc3cc4c(cc3c12)OCO4]

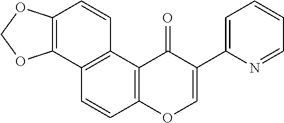 [SMILES: O=c1c(-c2ccccn2)coc2ccc3c4c(ccc3c12)OCO4]

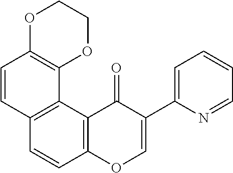 [SMILES: O=c1c(-c2ccccn2)coc2ccc3ccc4c(c3c12)OCCO4]

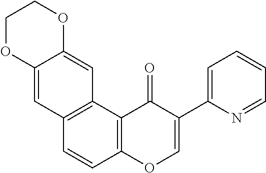 [SMILES: O=c1c(-c2ccccn2)coc2ccc3cc4c(cc3c12)OCCO4]

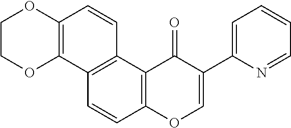 [SMILES: O=c1c(-c2ccccn2)coc2ccc3c4c(ccc3c12)OCCO4]

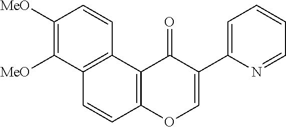 [SMILES: COc1ccc2c(ccc3occ(-c4ccccn4)c(=O)c32)c1OC]

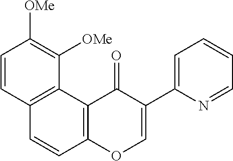 [SMILES: COc1ccc2ccc3occ(-c4ccccn4)c(=O)c3c2c1OC]

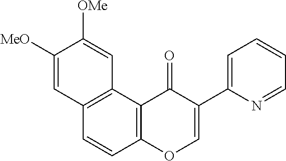 [SMILES: COc1cc2ccc3occ(-c4ccccn4)c(=O)c3c2cc1OC]

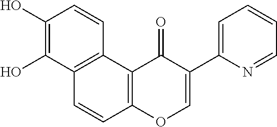 [SMILES: O=c1c(-c2ccccn2)coc2ccc3c(O)c(O)ccc3c12]

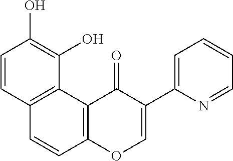 [SMILES: O=c1c(-c2ccccn2)coc2ccc3ccc(O)c(O)c3c12]

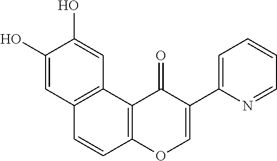 [SMILES: O=c1c(-c2ccccn2)coc2ccc3cc(O)c(O)cc3c12]

TABLE 4
Structure

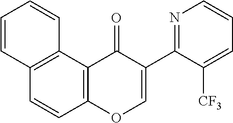 [SMILES: O=c1c(-c2ncccc2C(F)(F)F)coc2ccc3ccccc3c12]

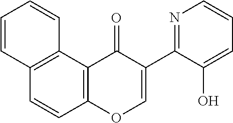 [SMILES: O=c1c(-c2ncccc2O)coc2ccc3ccccc3c12]

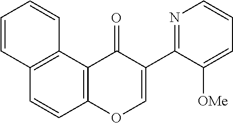 [SMILES: COc1cccnc1-c1coc2ccc3ccccc3c2c1=O]

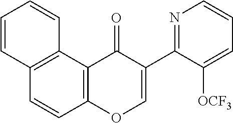 [SMILES: O=c1c(-c2ncccc2OC(F)(F)F)coc2ccc3ccccc3c12]

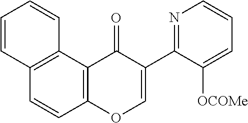

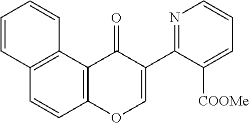 [SMILES: COC(=O)c1cccnc1-c1coc2ccc3ccccc3c2c1=O]

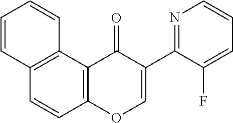 [SMILES: O=c1c(-c2ncccc2F)coc2ccc3ccccc3c12]

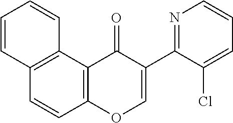 [SMILES: O=c1c(-c2ncccc2Cl)coc2ccc3ccccc3c12]

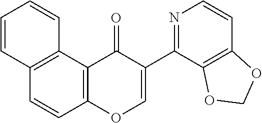 [SMILES: O=c1c(-c2nccc3c2OCO3)coc2ccc3ccccc3c12]

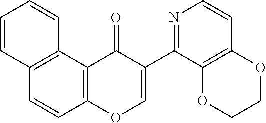 [SMILES: O=c1c(-c2nccc3c2OCCO3)coc2ccc3ccccc3c12]

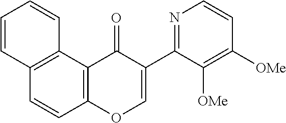 [SMILES: COc1ccnc(-c2coc3ccc4ccccc4c3c2=O)c1OC]

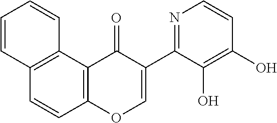 [SMILES: O=c1c(-c2nccc(O)c2O)coc2ccc3ccccc3c12]

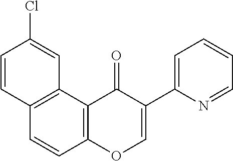 [SMILES: O=c1c(-c2ccccn2)coc2ccc3ccc(Cl)cc3c12]

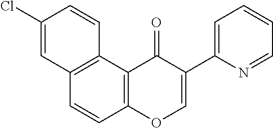 [SMILES: O=c1c(-c2ccccn2)coc2ccc3cc(Cl)ccc3c12]

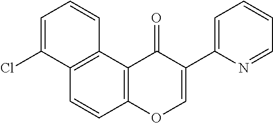 [SMILES: O=c1c(-c2ccccn2)coc2ccc3c(Cl)cccc3c12]

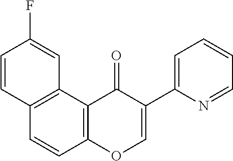 [SMILES: O=c1c(-c2ccccn2)coc2ccc3ccc(F)cc3c12]

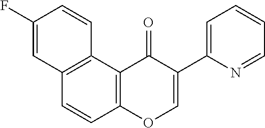 [SMILES: O=c1c(-c2ccccn2)coc2ccc3cc(F)ccc3c12]

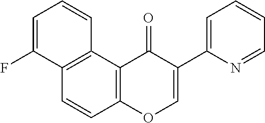 [SMILES: O=c1c(-c2ccccn2)coc2ccc3c(F)cccc3c12]

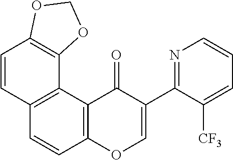 [SMILES: O=c1c(-c2ncccc2C(F)(F)F)coc2ccc3ccc4c(c3c12)OCO4]

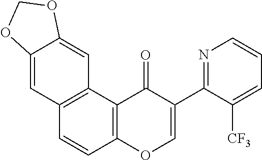 [SMILES: O=c1c(-c2ncccc2C(F)(F)F)coc2ccc3cc4c(cc3c12)OCO4]

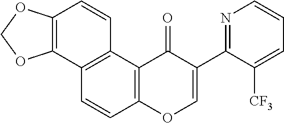 [SMILES: O=c1c(-c2ncccc2C(F)(F)F)coc2ccc3c4c(ccc3c12)OCO4]

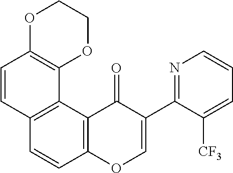 [SMILES: O=c1c(-c2ncccc2C(F)(F)F)coc2ccc3ccc4c(c3c12)OCCO4]

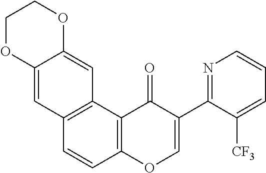 [SMILES: O=c1c(-c2ncccc2C(F)(F)F)coc2ccc3cc4c(cc3c12)OCCO4]

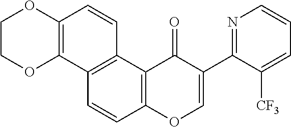 [SMILES: O=c1c(-c2ncccc2C(F)(F)F)coc2ccc3c4c(ccc3c12)OCCO4]

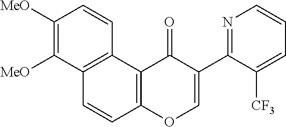 [SMILES: COc1ccc2c(ccc3occ(-c4ncccc4C(F)(F)F)c(=O)c32)c1OC]

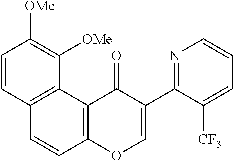 [SMILES: COc1ccc2ccc3occ(-c4ncccc4C(F)(F)F)c(=O)c3c2c1OC]

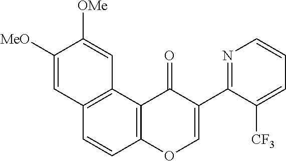 [SMILES: COc1cc2ccc3occ(-c4ncccc4C(F)(F)F)c(=O)c3c2cc1OC]

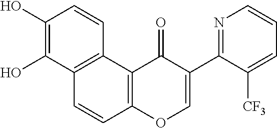 [SMILES: O=c1c(-c2ncccc2C(F)(F)F)coc2ccc3c(O)c(O)ccc3c12]

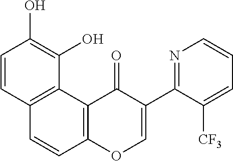 [SMILES: O=c1c(-c2ncccc2C(F)(F)F)coc2ccc3ccc(O)c(O)c3c12]

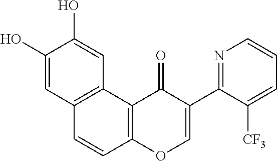 [SMILES: O=c1c(-c2ncccc2C(F)(F)F)coc2ccc3cc(O)c(O)cc3c12]

Methods for Measuring Activity

A preferred substance is preferably selected from compounds having the lipid modulating characteristics on the basis of at least one in vitro test. Choosing the best candidate may thus involve demonstrating a sufficient agonist effect, for example by the CALUX (chemically activated luciferase expression) or the EROD (ethoxy-resorufin deethylase) tests (He (2011)); Behnisch (2002)).

The effect of gradual decrease in the size of the sebaceous glands can be reproduced in animals by topical application of an active substance. The skin of the ears of a suitable animal, such as a rodent, including for example a mouse, rat or hamster ear may be chosen as a test site since it is known to contain abundant sebaceous glands and tissues.

It is also contemplated to measure the expression of the key enzymes of sebaceous lipogenesis in samples of skin tissue, for example, from the ear of a rodent, using RT-PCR and related techniques followed by a method to quantitate mRNA levels for example, either chip technologies or next generation gene sequencing technologies to track the resultant mRNA levels of said enzymes and also to track the factors which ensure promotion of the sebaceous stem cell as a possible target for lipid modulators.

One may also determine that the half-life of a substance in vivo is suitably short, following either topical or systemic administration, using analytical techniques such as HPLC or mass spectrometry or a combination thereof to measure concentrations of the substance or its metabolites in samples of blood or plasma taken from a treated mammal.

In addition, a substance may be tested for acting on particular cell types within a given time interval. Thus, in one embodiment of the method, said mammal is a mouse strain C57/BL6. According to this mode of execution, the ears said mice are treated topically, then harvested and the expression of CYP1A1 studied by immunohistochemistry using an antibody.

Formulations and Administration

The compound of the present invention is intended for pharmaceutical, dermatological and cosmetic use and may be formulated as a pharmaceutical composition and administered to a mammal, such as a human patient in a variety of forms adapted to a chosen route of administration, i.e. topically, intralesionally, orally, or subcutaneously. It should be understood that the invention is not limited by the chosen route of administration. The compound may be administered alone or in combination with one or more other therapeutic agents.

If desired, the compound can be administered directly without any excipients. However, in a typical embodiment the compound of the invention will be administered as a formulation in association with a pharmaceutically acceptable carrier. The choice of carrier will largely depend on factors such as the particular mode of administration, the effect of the carrier on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Gennaro (1995)).

The pharmaceutical compositions of the present invention comprise one or more compounds of the instant invention as an active ingredient or a pharmaceutically acceptable salt thereof, and may also contain a pharmaceutically acceptable carrier and optionally other therapeutic ingredients. The compositions include compositions suitable for topical, intralesional, rectal, parenteral (including subcutaneous, intramuscular, and intravenous), ocular (ophthalmic), pulmonary (nasal or buccal inhalation), nasal, intra-articular (i.e. in the joints) or oral administration, although the most suitable route in any given case will depend in part on the nature and severity of the conditions being treated and on the nature of the active ingredient. In certain embodiments, the pharmaceutical compositions of the present invention are suitable for topical, intralesional, parenteral, pulmonary, nasal, rectal or oral administration or, further, as an aerosol/powder for the nose, bronchi and/or lungs; an ointment or suppository for the rectum and/or the colon; or a formation for infiltration into the joints.

In other embodiments, the compound is topically applied to a subject. Topical application is especially appropriate for the treatment of acne, rosacea, excess sebum, oily skin or hair, and shiny or greasy looking skin. In certain embodiments, topical application refers to application of a compound, and optional carrier, directly to the skin and/or hair. The topical composition according to the present invention can be in the form of solutions, lotions, salves, creams, ointments, liposomes, sprays, gels, foams, roller sticks, or any other formulation routinely used in dermatology. In alternative embodiments, the composition is a patch, bandage or wound dressings.

In other embodiments, the compound is administered intralesionally. Such intralesional administration may take the form of injections given into the dermis or subcutaneous tissue. Intralesional administration may also be facilitated by use of a device, such as one containing micro-needles, to enhance drug penetration.

In yet other embodiments, the compound is administered orally. For oral administration, the compound may be formulated into solid or liquid preparations such as capsules, pills, tablets, lozenges, melts, powders, suspensions, or emulsions. Solid unit dosage forms can be capsules of the ordinary gelatin type containing, for example, surfactants, lubricants and inert fillers such as lactose, sucrose, and cornstarch or they can be sustained release preparations.

In some embodiments, the compound of Structure I is tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the active ingredient in an aqueous or nonaqueous pharmaceutically acceptable solvent, which may also contain suspending agents, sweetening agents, flavoring agents, and preservative agents as are known in the art.

In another embodiment, the compound Is administered parenterally. For parenteral administration, the compound may be administered as either a solution or a suspension. Examples of suitable pharmaceutical carriers for use in a solution or suspension are water, saline, dextrose solutions, fructose solutions, ethanol, or oils of animal, vegetative, or synthetic origin. Solutions or suspensions of these active compounds can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms. Injection solutions and suspensions can be prepared from sterile powders, granules, and tablets of the kind previously described. Formulations suitable for parenteral administration, such as, for example, by intra-articular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood recipient, and aqueous and non-aqueous sterile suspensions that can include suspending agents, solubilizers, thickening agents, stabilizers, and preservatives.

In other embodiments, compositions of the invention may be solid or semi-solid formulations which are suitable for use as cleansing soaps, gels or bars. These compositions are prepared according to the usual methods and may optionally contain additional excipients such as moisturizers, colorants, fragrances and the like.

In some embodiments, the compound will be formulated with a carrier suitable for administration directly to the skin or hair. The compound may be formulated for application to the hair in the form of aqueous, alcoholic or aqueous-alcoholic solutions, or in the form of creams, gels, emulsions or mousses, or alternatively in the form of aerosol compositions also comprising a propellant under pressure. The composition according to the invention can also be a hair care composition, and in particular a shampoo, a hair-setting lotion, a treating lotion, a styling cream or gel, a dye composition, a lotion or gel for preventing hair loss, etc. The amounts of the excipients in the various compositions according to the invention are those conventionally used in the fields considered.

In some embodiments, the compound is administered via transdermal route or dermal route, including patches. With respect to transdermal or dermal delivery routes of administration, methods for transdermal administration of drugs are disclosed in Remington's Pharmaceutical Sciences, Gennaro AR ed. 20th edition, 2000: Williams & Wilkins PA, USA. Creams, oils, sprays, other liquid formulations, dermal or skin patches are preferred means for transdermal delivery of the compounds of the invention. Patches preferably provide an absorption enhancer such as DMSO to increase the absorption of the compounds. Other methods for transdermal drug delivery are disclosed in U.S. Pat. Nos. 5,962,012 and 6,261,595, each of which is incorporated by reference in its entirety.

Preferred patches include those that control the rate of drug delivery to the skin. Patches may provide a variety of dosing systems including a reservoir system or a monolithic system, respectively. The reservoir design may, for example, have four layers: the adhesive layer that directly contacts the skin, the control membrane, which controls the diffusion of drug molecules, the reservoir of drug molecules, and a water-resistant backing. Such a design delivers uniform amounts of the drug over a specified time period, the rate of delivery has to be less than the saturation limit of different types of skin. The monolithic design, for example, typically has only three layers: the adhesive layer, a polymer matrix containing the compound, and a water-proof backing. This design brings a saturating amount of drug to the skin. Thereby, delivery is controlled by the skin. As the drug amount decreases in the patch to below the saturating level, the delivery rate falls.

In some embodiments, the compounds and compositions of the present invention are administered to men. In other embodiments, the compounds and compositions of the present invention are administered to women. In certain embodiments, the compounds and compositions of the present invention are administered to women of child-bearing age. In some other embodiments, the compounds and compositions are administered to women who are pregnant. In certain other embodiments, the compounds and compositions of the present invention are administered to children.

In certain embodiments, the compounds and compositions of the present invention are administered to children under the age of 18 years old. In further embodiments, the compounds and compositions of the present invention are administered to children under the age of 16 years old. In alternative embodiments, the compounds and compositions of the present invention are administered to children under the age of 14 years old, under the age of 12 years old or under the age of 10 years old. In some embodiments, the methods include administering the compounds and compositions to pre-pubescent children.

Kits providing a unit dosage of the compounds and compositions set forth herein are contemplated as within the present invention. Kits providing many unit dosages of the compounds and compositions set forth herein are also contemplated as within the present invention. Still further, kits providing several unit dosages of the compounds and compositions set forth herein are contemplated as within the present invention. In some embodiments, the kits of the present invention include a unit dosage of a pharmaceutical composition of a compound set forth herein. In certain embodiments, the kits of the present invention include many unit dosages of a pharmaceutical composition of a compound set forth herein. In certain other embodiments, the kits of the present invention include a unit dosage of a pharmaceutical composition set forth herein.

Dosage

The dose and dosing regimens of the compound of the invention may be adjusted to provide the optimum desired response in accordance with methods and practices well known in the therapeutic arts. For example, a single bolus dose may be administered or several divided doses may be administered over time. The dose may also be proportionally reduced or increased as indicated by the exigencies of the therapeutic situation. The appropriate dosing regimen, the amount of each dose administered and/or the intervals between doses will depend upon a number of factors, including: the compound, the type of pharmaceutical composition, the characteristics of the subject in need of treatment and the severity of the condition being treated.

The dose of the compound will vary, but as a general guideline for dermatological administration, the compound will be present in a dermatologically acceptable formulation in an amount of from about 0.01 to 50 w/w %, and more typically from about 0.1 to 10 w/w %. In some embodiments, the formulation may be applied to the affected area from 1 to 4 times daily. A “dermatologically acceptable formulation” is one that may be applied to the skin or hair and will allow the drug to diffuse to the site of action. In some embodiments, the amounts effective for topical formulation will depend on the severity of the disease, disorder or condition, previous therapy, the individual's health status and response to the drug. In certain other embodiments, the dose is in the range from 0.001% by weight to about 60% by weight of the formulation.

The skilled artisan can also be expected to readily determine the maximum tolerable dose, the therapeutically effective amount which provides a detectable therapeutic benefit to a patient, and the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

The determination of optimal dosages for a particular patient is well-known to those skilled in the art. Certain non-limiting examples of pharmaceutically acceptable vehicles suitable for topical administration include propylene glycol: transcuta nol: ethanol (20:20:60, v/v/v) and propylene glycol:ethanol (30:70, v/v). In some embodiments, the compound of Structure I may be present at concentrations of between about 1.5% to about 2.0% (w/v)

In one embodiment, the compound of the present invention is administered in dosages from about 0.1 to 1000 mg, 1 to about 1000 mg, 100 to about 500 mg or about 1 to about 100 mg. In a further embodiment, the compound of the present invention is administered at a dose of 0.05 to about 100 mg, and more preferably from about 0.1 to about 100 mg, per day. In a preferred embodiment, the dosage is about 0.1 mg to about 70 mg per day. In another embodiment, in choosing a regimen for patients, the compound of the present invention is administered starting with a dosage of from about 2 to about 70 mg per day and when the condition is under control the dosage is reduced to as low as from about 0.1 to about 10 mg per day. In a further embodiment, e.g., in the treatment of adult humans, dosages from about 0.05 to about 100 mg, preferably from about 0.1 to about 100 mg, per day may be used.

The exact dosage will depend upon the mode of administration, the compound of the invention involved, on the therapy desired, form in which administered, the subject to be treated and the body weight of the subject to be treated, and the preference and experience of the physician or veterinarian in charge.

In some embodiments, the compounds and compositions set forth herein are administered once daily. In other embodiments, the compounds and compositions set forth herein are administered twice a day. In other embodiments, the compounds and compositions set forth herein are administered three times a day. In other embodiments, the compounds and compositions set forth herein are administered four times a day. In other embodiments, the compounds and compositions set forth herein are administered five times a day.

In some embodiments, the compounds and compositions set forth herein are administered weekly. In other embodiments, the compounds and compositions set forth herein are administered monthly. In other embodiments, the compounds and compositions set forth herein are administered twice a week. In other embodiments, the compounds and compositions set forth herein are administered three times a week. In other embodiments, the compounds and compositions set forth herein are administered four times a week. In other embodiments, the compounds and compositions set forth herein are administered five times a week. In other embodiments, the compounds and compositions set forth herein are administered six times a week. In other embodiments, the compounds and compositions set forth herein are administered seven times a week. In other embodiments, the compounds and compositions set forth herein are administered eight times a week. In other embodiments, the compounds and compositions set forth herein are administered nine times a week. In other embodiments, the compounds and compositions set forth herein are administered ten times a week. In other embodiments, the compounds and compositions set forth herein are administered eleven times a week. In other embodiments, the compounds and compositions set forth herein are administered twelve times a week. In other embodiments, the compounds and compositions set forth herein are administered thirteen times a week. In other embodiments, the compounds and compositions set forth herein are administered fourteen times a week.

In a further embodiment, the compound and compositions set forth herein are administered so as to minimize the systemic bioavailability of the lipid modulating compound in patients. In some embodiments, the lipid modulating compounds have reduced average systemic bioavailabllity. In other embodiments, reduced average systemic bioavailability is when the average systemic bioavailability is less than 30%, less than 25%, less than 15%, less than 10%, less than 5%, less than 4%, less than 3%, less than 2%, and less than 1% as compared to an immediate or extended release formulation or a conventional topical formulation having an equivalent amount of the lipid modulating compound.

Co-administration

In further embodiments of the invention, the compound is co-administered with other agents in order to enhance or complement the desired therapeutic effect or to minimize potential side effects. Non-limiting examples of such embodiments are described below. Acyl-CoA cholesterol acyl transferase (ACAT) inhibitors were initially evaluated for the treatment of elevated serum cholesterol. It was subsequently discovered that these compounds decrease sebum production (U.S. Pat. No. 6,133,326). Any such ACAT inhibitor can be co-administered with the compound(s) of Structure I to decrease sebum production, alleviate oily skin, etc.

Stearoyl-CoA Desaturase-1 (SCD-1) inhibitors are being developed for sebum reduction and the treatment of acne. Any such SCD-1 inhibitor can be co-administered with the compound(s) of Structure I to decrease sebum production, alleviate oily skin, etc.

Topical retinoids are used to treat acne by normalizing follicular keratinization, but do not effectively reduce sebum production. In an embodiment of the invention, a compound of Structure I is co-administered with a retinoid in order to decrease sebum production and to treat acne or seborrhea. Exemplary retinoids suitable for coadministration include, but are not limited to, etretinate, tretinoin, retinal, retinyl palmitate, adapalene, tazarotene, and aliretinoin.

Benzoyl peroxide has been a mainstay in the treatment of acne for many decades and works, at least in part, by reducing skin colonization with Propionobacterium acnes. In an embodiment of the invention, the compound(s) of Structure I is co-administered with benzoyl peroxide to enhance the treatment of acne.

Antibiotics, such as members of the tetracycline family (including minocycline and doxycycline), clindamycin, erythromycin, and dapsone have been used to treat acne. The antibiotic reduces or eradicates the microorganism, Propionbacterium acnes, leading to a reduction in the patient's acne. The compound(s) of Structure I can be co-administered with any antibiotic suitable for the treatment of acne.

Estrogen and progesterone have each been shown to decrease sebum production. These compounds, or any synthetic agonist of such compounds, may be co-administered with the compound(s) of Structure I in order to decrease sebum production.

Anti-androgens have been shown to decrease sebum production. These compounds, or any synthetic anti-androgen, may be co-administered with the compound(s) of Structure I in order to decrease sebum production.

As used in this application, the terms “co-administered” or “co-administration” refer to a dosing regimen where the compound of Structure I is administered with a second therapeutic agent, typically having a differing mechanism of action, to promote a desired result. It should be understood that “co-administration” is not limited by the route(s) of administration and can refer to simultaneous dosing, dosing at different times during a single day, or even dosing on different days. The compounds can be administered separately or can be combined into a single formulation (i.e. fixed combination).

In another embodiment, the medicinal and cosmetic formulations containing the compound and any additional therapeutic agents will typically be packaged for retail distribution (i.e. an article of manufacture or a kit). Such articles will be labeled and packaged in a manner to instruct the patient how to use the product. Such instructions will include the condition to be treated, duration of treatment, dosing schedule, etc. The compound(s) of Structure I may also be admixed with any inert carrier and utilized in laboratory assays in order determine the concentration of the compounds within the serum, urine, etc., of the patient as is known in the art. The compound may also be used as a research tool.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention. The following examples and biological data are being presented in order to further illustrate the invention. This disclosure should not be construed as limiting the invention in any manner.

For all of the foregoing embodiments, each embodiment disclosed herein is contemplated as being applicable to each of the other disclosed embodiments. Those skilled in the art will readily appreciate that the specific Experimental Details which follow are only illustrative of the invention as described more fully in the claims which follow thereafter.

### EXPERIMENTAL DETAILS

The invention will be understood more clearly by those skilled in the art through the description hereinafter of several specific experiments, with reference to the corresponding examples and to the accompanying figures as follows:

### Example 1

### Synthesis of Compounds

NMR spectra were recorded on Bruker Avance 400 MHz for ¹H NMR and 100 MHz for ¹³C NMR. LCMS were taken on a single quadrupole Mass Spectrometer using Shimadzu LCMS 2010 (Column: sepax ODS 50×2.0 mm, 5 um) or Agilent 1200 H PLC, 1956 MSD (Column: Shim-pack XR-ODS 30×3.0 mm, 2.2 um) operating in ES (+) ionization mode. Chromatographic purifications were by flash chromatography using 100200 mesh silica gel. Anhydrous solvents were pre-treated with 3A Molecular Sieves column before use. All commercially available reagents were used as received unless otherwise stated.

Compound B

General Procedure for Preparation of intermediate B2

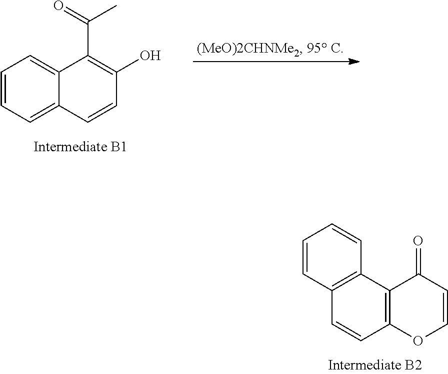

To a solution of Intermediate B1 (10.00 g, 53.70 mmol, 1.00 eq) in Xylene (100.00 ml) was added (MeO)₂CHNMe₂ (31.99 g, 268.50 mmol, 5.00 eq) at 25° C., then the mixture was stirred at 95 UC for 16 hr. TLC (Petroleum Ether/Ethyl Acetate 5:1) showed that starting material was consumed. The mixture was concentrated to give crude product as a black oil, which was purified by chromatography (Petroleum Ether/Ethyl Acetate 50:1 to 20:1) to give Intermediate B2 (6.00 g, 27.52 mmol, 51.25% yield, 90% purity) as a yellow solid.

TLC Information (Eluent: Petroleum Ether/Ethyl Acetate 3:1)

R_{f}(B1)=0.35

R_{f}(B2)=0.49

¹H NMR: (CDCl₃, 400 MHz): δ 10.04 (d, J=8.4 Hz, 1H), 8.08 (d, .1=8.0 Hz 1H), 7.91-7.88 (m, 2H), 7.76 (t, J=8 Hz, 1H), 7.62 (t, J=8 Hz, 1H), 7.50 (d, J=8 Hz, 1H), 6.52 (d, J=5.6 Hz, 1H)

General Procedure for Preparation of Intermediate B4

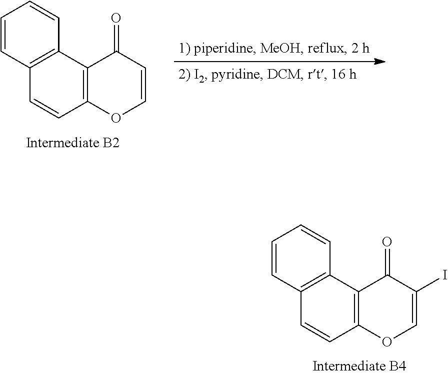 [SMILES: O=c1ccoc2ccc3ccccc3c12 ; O=c1c(I)coc2ccc3ccccc3c12]

To a solution of Intermediate B2 (6.00 g, 30.58 mmol, 1.00 eq) in MeOH (200.00 ml) was added Piperidine (44.27 g, 519.88 mmol, 17.00 eq) at 25° C., then the mixture was stirred at 65° C. for 4 hr. The mixture was concentrated to give red oil. Dichloromethane (200 mL) and Pyridine (4.84 g, 61.16 mmol, 2.00 eq) were added, then a solution of I₂ (38.81 g, 152.91 mmol, 5.00 eq) in Dichloromethane (150 mL) was added drop-wise. The mixture was stirred at 25° C. for 16 hr. TLC (Petroleum Ether/Ethyl Acetate 5:1) showed starting material was consumed. Aq. Na₂S₂O₃ (500 mL) was added, stirring continued at 25° C. for 30 min, the Dichloromethane layer was collected and concentrated to give black oil, which was purified by chromatography (Petroleum Ether/Ethyl Acetate 50:1 to 20:1) to give Intermediate 84 (6.00 g, 16.76 mmol, 54.82% yield, 90% purity) as yellow solid, confirmed by ¹HNMR.

TLC Information (Eluent: Petroleum Ether/Ethyl Acetate 5: 1)

R_{f}(B2)=0.45

R_{f}(B4)=0.50

¹H NMR_{:} (CDCl₃, 400 MHz): δ 9.92 (d, J=8.4 Hz, 1H), 8. 27 (s, 1H), 8.02 (d, J=8 Hz, 1H), 7.82 (d, J=8.4 Hz, 1H), 7.72-7.68 (m, 1H), 7.58-7.54 (m, 1H), 7.41. (d, J=8.8 Hz, 1H)

General Procedure for Preparation of Compound B

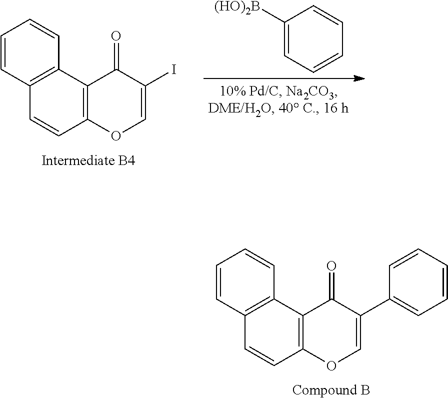 [SMILES: O=c1c(I)coc2ccc3ccccc3c12 ; OB(O)c1ccccc1 ; O=c1c(-c2ccccc2)coc2ccc3ccccc3c12]

The mixture of Intermediate B4 (2.00 g, 6.21 mmol, 1.00 eq), Phenyl Boronic Acid (908.62 mg, 7.45 mmol, 1.20 eq), Na₂CO₃ (1.97 g, 18.63 mmol, 3.00 eq) and Pd/C (450.00 mg, 621.00 umol, 0.10 eq) in H₂O (30.00 mL) and Diemthoxyethane (30.00 mL) was stirred at 40° C. for 16 hr under N₂ protection. TLC (Petroleum Ether/Ethyl Acetate 5:1) showed SM consumed. The mixture was filtered, and the filtrate was washed with water (100 ml), the organic layer was concentrated to give crude product as gray solid. The crude was purified by chromatography (Petroleum Ether/Ethyl Acetate 100:1 to 50:1) to give Compound B (1.00 g, 3.67 mmol, 59.14% yield, 100% purity) as white solid, LCMS showed 100% purity.

TLC Information (Eluent: Petroleum Ether/Ethyl Acetate=5:1)

R_{f}(B4)=0.50

R_{f}(Compound B)=0.55

LCMS: t=3.296 min, MS cal.: 272.1, [M+1]⁺=273.1, [a. mobile phase (solvent A: H₂O containing 0.0375% TFA; solvent B: Acetonitrile containing 0.018% TFA); gradient: 0.00: 90% A; 0.40: 90% A; 3.40: 0% A; 3.85: 0% A; 3.86: 90% A; 4.50: 90% A ; flow rate: 0.8 mi./min; column: Venusil XBP-C18; column temperature: 50° C].

¹H NMR: (CDCl₃, 400 MHz): δ 10.04 (d, J=8.4 Hz, 1H), 8.02 (d, J=8.8 Hz 1H), 7.98 (s, 1H), 7.84 (d, J=8 Hz, 1H), 7.70-7.66 (m, 1H), 7.57-7.53 (m, 3H), 7.46-7.32 (m, 4H)

Compound F

General Procedure for Preparation of Compound F

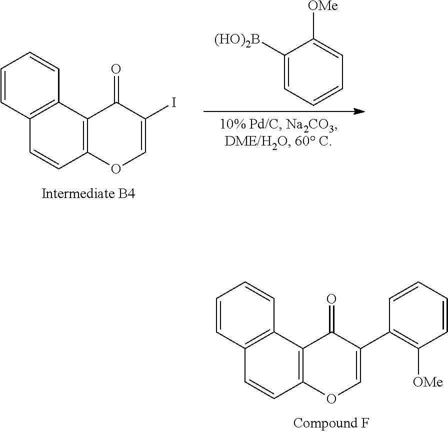 [SMILES: O=c1c(I)coc2ccc3ccccc3c12 ; COc1ccccc1B(O)O ; COc1ccccc1-c1coc2ccc3ccccc3c2c1=O]

10% Pd/C (3.00 g) was added to a solution of intermediate B4 (3.50 g, 10.8 mmol, 1.00 eq), 2-methoxy phenyl boronic acid (1.82 g, 11.9 mmol, 1.10 eq) and Na₂CO₃ (3.46 g, 32.6 mmol, 3.00 eq) in DME (50.0 mL) and H₂O (50.0 mL) at 60° C. under N₂. The reaction was stirred at 60° C. for 16 h. LCMS showed the reaction was completed. The reaction mixture was filtered and the filtrate was concentrated. The mixture was then washed with MeOH (5 ml×3) filtered and the filtrate was concentrated. Compound F (2.50 g, 86% yield) was obtained as a white solid.

¹H NMR: (CDCl₃, 400 MHz): δ 10.10 (d, J=8.0 Hz, 1H), 8.09 (d, J=8.0 Hz, 1H), 8.02 (s, 1H), 7.91 (d, J=8.0 Hz, 1H), 7.73 (t, J=8.0 Hz, 1H), 7.61 (t,J=6.0 Hz, 1H), 7.51 (d, J=8.0 Hz, 1H), 7.40-7.37 (m, 1H), 7.06 (s, 1H), 7.03-7.01 (m, 2H), 3.81 (s, 3H).

LCMS: t=2.96 min, MS cal.:302.1, [M+1]⁺=303.1, [a. mobile phase (solvent A: H₂O containing 0.0375% TFA; solvent B: Acetonitrile containing 0.018% TFA); gradient: 0.00: 90% A; 0.40: 90% A; 3.40: 0% A; 3.85: 0% A; 3.86: 90% A; 4.50: 90% A ; flow rate: 0.8 mL/min; column: Venusil XBP-C18; column temperature: 50° C].

Compound G

General Procedure for Preparation of Compound G

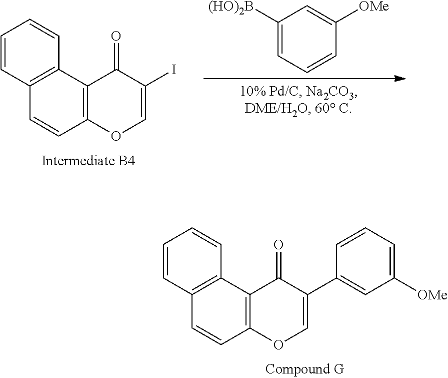 [SMILES: O=c1c(I)coc2ccc3ccccc3c12 ; COc1cccc(B(O)O)c1 ; COc1cccc(-c2coc3ccc4ccccc4c3c2=O)c1]

10% Pd/C (3.00 g) was added to a solution of intermediate B4 (3.00 g, 9.31 mmol, 1.00 eq), 3-methoxy phenylboronic acid (1.56 g, 10.2 mmol, 1.10 eq) and Na₂CO₃ (2.96 g, 27.9 mmol, 3.00 eq) in DME (75.0 mL) and H₂O (75.0 mL) at 60° C. under N₂. The reaction was stirred at 60° C. for 16 h. LCMS showed the reaction was complete. The reaction mixture was filtered and the filtrate was concentrated. The mixture was washed with water (15 mL×3). The reaction mixture was filtered and the filtrate was concentrated. Compound G (2.50 g, 86% yield) was obtained as a white solid.

¹H NMR: (CDCl₃, 400 MHz): δ 10.11 (d, J=8.0 Hz, 1H), 8.10 (d, J=8.0 Hz, 1H), 8.06 (s, 1H), 7.92 (d, J=8.0 Hz, 1H), 7.76 (t, J=8.0 Hz, 1H), 7.65 (t, J=6.0 Hz, 1H), 7.54 (d, J=8.0 Hz, 1H), 7.39 (t, J=6.0 Hz, 1H), 7.19-7.16 (m, 2H), 6.92-6.89 (m, 1H), 3.87 (s, 3H).

LCMS: t=3.07 min, MS cal.: 302.1, [M+1]⁺=303.1, [a. mobile phase (solvent A: H₂O containing 0.0375% TFA; solvent B: Acetonitrile containing 0.018% TFA); gradient: 0.00: 90% A; 0,40: 90% A; 3.40: 0% A; 3.85: 0% A; 3.86: 90% A; 4.50:90% A ; flow rate: 0.8 mL/min; column: Venusil XBP-C18; column temperature: 50° C.).

Compound H

General Procedure for Preparation of Compound H

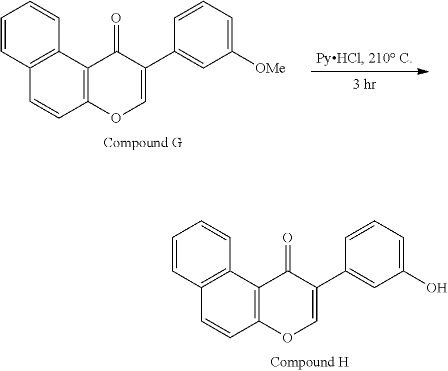 [SMILES: COc1cccc(-c2coc3ccc4ccccc4c3c2=O)c1 ; O=c1c(-c2cccc(O)c2)coc2ccc3ccccc3c12]

Pyridine.HCl (11.4 g, 99.2 mmol, 20.00 eq) was added to compound 0 (1.50 g, 4.96 mmol, 1.00 eq) at 25° C. The reaction was stirred at 210° C. for 3 h. LCMS showed the reaction was completed. The mixture was poured into water (50 mL) and stirred for 10 min. The mixture was washed with ethyl acetate (10 mL×3). The reaction mixture was filtered and the cake was concentrated. Compound H (1.20 g, 81% yield) was obtained as a white solid.

¹H NMR: (CDCl₃, 400 MHz): δ 9.99 (d, J=8.0 Hz, 1H), 9.51 (s, 1H), 8.57 (s, 1H), 8.37 (d, J=8.0 Hz, 1H), 8.14 (d, J=8.0 Hz, 1H), 7.82-7.75 (m, 2H), 7.73-7.69 (m, 1H), 7.27 (t, J=8.0 Hz, 1H), 7.08 (S, 1H), 7.04 (d, J=8.0 Hz, 1H), 6.83 (d, J=8.0 Hz, 114).

LCMS: t=2.56 min, MS cal.: 288.1, [M+1]⁺=289.1, [a. mobile phase (solvent A: H₂O containing 0.0375% TFA; solvent B: Acetonitrile containing 0.018% TFA); gradient: 0.00: 90% A; 0.40: 90% A; 3.40: 0% A; 3.85: 0% A; 3.86: 90% A; 4.50: 90% A ; flow rate: 0.8 mL/min; column: Venusil XBP-C18; column temperature: 50° C].

Compound I

General Procedure for Preparation of Compound I

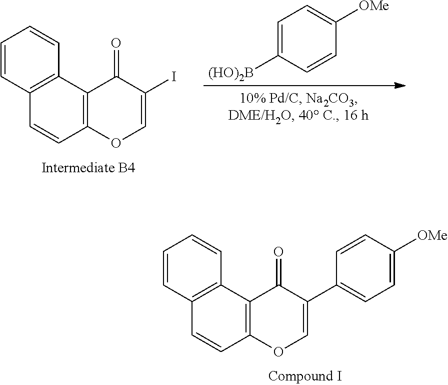 [SMILES: O=c1c(I)coc2ccc3ccccc3c12 ; COc1ccc(B(O)O)cc1 ; COc1ccc(-c2coc3ccc4ccccc4c3c2=O)cc1]

10% Pd/C (1.00 g) was added to a solution of intermediate B4 (1.20 g, 3.73 mmol, 1.00 eq), 4-methoxy phenylboronic acid (680.17 mg, 4.48 mmol, 1.20 eq) and Na₂CO₃ (1.19 g, 11.19 mmol, 3.00 eq) in DME (15.0 mL) and H₂O (15.0 mL) at 25° C. under N₂. The reaction was stirred at 60° C. for 16 h. TLC (Petroleum ether/Ethyl acetate 5:1) showed the reaction was completed. The reaction mixture was filtered and the filtrate was concentrated. The mixture was washed with MeOH (5 mL×3). The reaction mixture was filtered and the filtrate was concentrated. Compound 1(1.00 g, 86% yield) was obtained as a white solid.

¹H NMR: (CDCl₃, 400 MHz): δ 10.02 (d, J=12.0 Hz, 1H), 8.19 (d, J=8.0 Hz, 1H), 8.04 (s, 1H), 7.92 (d, 1=8.0 Hz, 1H), 7.75 (t, J=8.0 Hz, 1H), 7.62 (t, J=6.0 Hz, 1H), 7.57-7.52 (m, 3H), 7.01 (d, J=8.0 Hz, 1H), 3,86 (s, 3H).

LCMS: t=3.07 min, MS cal.: 302.1, [M+1]⁺=303.1, [a. mobile phase (solvent A: H₂O containing 0.0375% TFA; solvent B: Acetonitrile containing 0.018% TFA); gradient: 0.00: 90% A; 0.40: 90% A; 3.40: 0% A; 3.85: 0% A; 3.86: 90% A; 4.50: 90% A ; flow rate: 0.8 mL/min; column: Venusil XBP-C18; column temperature: 50° C].

Compound J

General Procedure for Preparation of Compound J

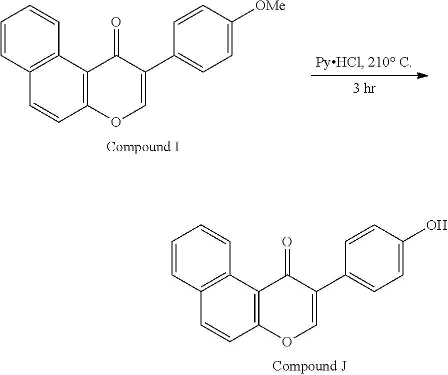 [SMILES: COc1ccc(-c2coc3ccc4ccccc4c3c2=O)cc1 ; O=c1c(-c2ccc(O)cc2)coc2ccc3ccccc3c12]

Pyridine.HCl (11.47 g, 99.23 mm ol, 20.00 eq) was added to compound I (1.50 g, 4.96 mmol, 1.00 eq) at 25° C. The reaction was stirred at 210° C. for 3 h. TLC (petroleum ether/ethyl acetate 5:1) showed the reaction was completed. The mixture was poured into water (50 mL) and stirred for 10 min. The mixture was washed with ethyl acetate (10 mL×3). The reaction mixture was filtered and the cake was concentrated, Compound J (1.20 g, 3.21 mmol, 82% yield) was obtained as a white solid.

¹H NMR: (CDCl₃, 400 MHz): δ 9.95 (d, J=8.0 Hz, 1H), 9.54 (s, 1H), 8.47 (s, 1H), 8.31 (d, J=6.0 Hz, 1H), 8.08 (d, J=8.0 Hz, 1H), 7.76-7.70 (m, 2H),7.65 (t, J=8.0 Hz, 1H), 7.75 (d, J=8.0 Hz, 1H), 6.81 (t, J=8.0 Hz, 1H).

LCMS: t=2.56 min, MS cal.: 288.1, [M+1]⁺=289.1, [a. mobile phase (solvent A: H₂O containing 0.0375% TFA; solvent B: Acetonitrile containing 0.018% TFA); gradient: 0.00: 90% A; 0.40: 90% A; 3.40: 0% A; 3.85: 0% A; 3.86: 90% A; 4.50: 90% A ; flow rate: 0.8 mL/min; column: Venusil XBP-C18; column temperature: 50° C.]

### Example 2

### Activation of the Aryl Hydrocarbon Receptor by Lipid Modulating Compounds in HEP G2 cells

AhR activation is measured in HEP G2 cells (hepatocytes, Hep G2 is a human liver carcinoma cell line) stably transfected with the lentivector plox-XRE TATA-Luc. The Hep G2 cells are cultured in a growing media consisting of DM EM (Glbco)+10% fetal bovine serum+penicillin+streptomycin. At D0, the Hep G2 cells are seeded into 12-well plates in proportions of approximately 30,000 cells/cm². After 24 h, the medium is replaced with fresh medium and the cells are transduced with the lentivector plox-XRE TATA-Luc. After 48 h, the cells are subcultured and maintained in culture, and tested for their reactivity to 3-phenyl-1H-benzo[f]chromen-1-one. The tests are carried out using the luciferase reporter assay system kit from Promega. At D0, the cells are seeded at a density of approximately 60% confluence, and then treated, at D1, with the test substance diluted to various concentrations in the appropriate culture medium. At D2, the cells are lysed in CLB buffer, and the lysate is clarified by centrifugation for 5 min at 10 000 g. The luciferase activity is measured in 20 microliters of lysate as recommended by the supplier, using the luminoskan luminometer (Thermo). After treatment for 24 h with 1-10 uM of 3-phenyl-1H-benzo[f]chromen-1-one, a significant induction of the luciferase activity is observed in the Hep G2-plox-XRE TATA-Luc cells. In subsequent experiments adapted to either 96 well or 384 well format compounds are screened in “hit mode” in triplicate using a standard concentration of 1 uM per compound and having 3-phenyl-1H-benzo[f]chromen-1-one control set of wells. Hits thus identified are reformatted in dose response mode in 384 well plates (dose range 0.1 nM to 10 uM), again in triplicates with 3-phenyl-1H-benzo[f]chromen-1-one (dose range 0.1 nM to 10 uM) as control. Data is processed and ECₛₒ's can be calculated from standard data monitoring software.

### Example 3

### Activation of the Aryl Hydrocarbon Receptor in Human Skin Cells

AhR activation is also measured in human skin cells such as normal human keratinocytes (NHK cells) or A431 epidermoid cells stably transfected with the lentivector plox-XRE TATA-Luc. The NHK cells are cultured in a specific keratinocyte SFM medium (Glbco)+penicillin+streptomycin. At D0, the NHK cells are seeded Into 6-well plates in a proportion of approximately 15,000 cells/cm². After 24 h, the medium is replaced with fresh medium and the cells are transduced with the lentivector plox-XRE TATA-Luc. After 48 h, the cells are subcultured and maintained in culture, and tested for their reactivity to 3-phenyl-1H-benzo[f]chromen-1-one. The tests are carried out using the luciferase reporter assay system kit from Promega. At D0, the cells are seeded at a density of approximately 60% confluence, and then treated, at D1, with the test substance diluted to various concentrations in the appropriate culture medium. At D2, the cells are lysed in CLB buffer, and the lysate is clarified by centrifugation for 5 min at 10 000 g. The luciferase activity is measured in 20 microliters of lysate as recommended by the supplier, using the luminoskan luminometer (Thermo). After treatment for 24 h with 1-10 uM of 3-phenyl-1H-benzo[f]chromen-1-one, a significant induction is observed in the NHK-plox-XRE TATA-Luc cells. In subsequent experiments adapted to either 96 well or 384 well format compounds are screened in “hit mode” in triplicate using a standard concentration of 1 uM per compound and having a 3-phenyl-1H-benzo[f]chromen-1-one control set of wells containing 3-phenyl-1H-benzo[f]chromen-1-one. Hits thus identified are reformatted in dose response mode in 384 well plates (dose range 0.1 nM to 10 uM), again in triplicates. Data is processed and ECₛₒ's can be calculated from standard data monitoring software.

### Example 4

### Reduction of Secreted Triglyceride Levels from Human Adipocytes

The Human AdipoRed™ Assay allows compounds to be examined rapidly for their ability to function in intact human adipocyte cells and inhibit the secretion of triglycerides in an analogous way to the secretion of sebum from human sebocytes. Human adipocyte cells are received as pre-adipocytes and then differentiated for 5 days in a 384 well format. Compound is then added at a standard concentration of 1 uM concentrations for 6 days. The production of triglycerides is then assessed by a unique dye (AdipoRed™, a proprietary formulation of Nile Red from Lonza Walkersville, Inc., www.lonza.com, Document # AA-1038-7 04/11, Walkersville, Md. 21793-0127 USA) which specifically binds to secreted triglycerides generating a fluorescent signal in a lipophilic environment. The lipophilic AdipoRed™ specifically partitions into the fat droplets, binding to triglycerides and the latter are simply quantitated by measuring fluorescence at 572 nm. Compounds are also tested in cell based assays for viability using standard methods well known in the art such as assays using the tetrazolium dye MIT, to distinguish between selective inhibition of lipid synthesis versus secondary decreases in levels due to non-specific cytotoxicity.

More specific cell based assays also employ mass spectrometry methods to evaluate the exact lipid profile in the presence of such modulators, and are well established by those skilled in the art (Camera (2010)).

### Example 5

### Reduction of Secreted Triglyceride Levels from Human Sebocytes

The essential methodology for culture and handling of SZ-95 cells or 5E13-1 cells is similar to that described for human adipocytes in Example 4. Evaluation of lipid secretion is achieved using AdipoRed™ as described in Example 4.

More specific cell based assays also employ mass spectrometry methods to evaluate the exact lipid profile in the presence of such modulators, and are well established by those skilled in the art (Camera (2010)). Other assays to measure total synthesis use C14 labelled acetate conversion into lipids. Inhibition of synthesis is reflected by decreased C14 incorporation

### Example 6

### Reduction of Secreted Triglyceride Levels from Human Hepatocytes

The essential methodology for culture and handling of HEP-G2 cells has been described in Examples 2 and B. Evaluation of lipid secretion is achieved using AdipoRed as described in Example 3.

More specific cell based assays also employ mass spectrometry methods to evaluate the exact lipid profile in the presence of such modulators, and are well established by those skilled in the art (Camera (2010)).

### Example 7

### Reduction of Secreted Triglyceride Levels from Human Keratinocytes

The essential methodology for culture and handling of NHK cells has been described in Example 3. Evaluation of lipid secretion is achieved using AdipoRed as described in Example 4.

More specific cell based assays also employ mass spectrometry methods to evaluate the exact lipid profile in the presence of such modulators, and are well established by those skilled in the art (Camera (2010)).

### Example 8

### Induction of CYP1A1 and/or SCD-1 In HEP G2 Cells as Measured by mRNA (CYP1A1 and SCD-1 mRNA Assay)

HepG2 cells (American Type Culture Collection (ATCC), Manassas, Va.) were cultured using Eagle's minimal essential medium (ATCC), supplemented with 10% fetal calf serum (Invitrogen Life Technologies). The cells were cultured continuously until used in experiments (similar to Examples 2 and 3). Celts were plated at a seeding density of 3.8×10⁵ cells per well on 6-well plates (Corning Costar, N.Y., U.S.A.). Cells were allowed to attach for 24 h in a 37° C. humidified incubator with a 5% CO2 atmosphere. Media was replaced with that containing test agents or vehicle (0.1% dimethylsuiphoxide) and cells incubated for a further 24 h. Following the 24 h incubation with test agents, media was removed and RNA prepared using the Purelink RNA mini kit (ThermoFischer, Carlsbad, Calif.). Nucleic acid concentration was determined using a Nanodrop™ spectrophotometer (ThermoScientific) by measuring the OD at 260 nm and RNA purity was evaluated using the OD 260/280 nm ratio, 1 μg RNA was reverse transcribed using SuperScript®VILO™ (ThermoFischer) with cDNA synthesis performed using the following parameters: 10min at 25° C., 2 h at 42° C., 5min at 85° C. (deactivation) followed by a completion temperature de C. The resultant cDNA was diluted 20-fold with nucleic acid-free molecular biology grade water to a concentration of 100 ng/μl and 1 μl diluted cDNA (100 ng) used for RT-PCR. Each sample (1 μl) was pipetted into a 96-well optical reaction plate (Applied Biosystems, Carlsbad, Calif.). Reagent mix was made using the TaqMan Master Mix (AppliedBiosystems) and 19.0 μl of reagent mix containing PCR primers and TaqMan probes was added to each well. PCR primer and TaqMan probe sequences were purchased as customized mixes (ThermoFischer, Carlsbad, Calif.). RT-PCR was performed on the ABI Prism 7900 (Applied Biosystems) with the following parameters: 2 min at 50° C. (initial step), 10min at 95° C., and 40 cycles of 15 s melting at 95° C. then 1 min annealing/extending at 60° C. Results were analyzed by calculating the fold change of CYP1A1 mRNA levels normalized to 18S rRNA (ΔΔCₜ) in compound treated cells or tissues compared to vehicle levels.

The results are shown in Table 5 below. In this assay several compounds, including compound B are potent inducer of CYP1A1 mRNA mRNA at 10 uM. When these results are combined with other in vitro and in vivo results, AhR agonists with a different (improved) profile compared to 3-Phenyl-1H-benzo[f]chromen-1-one (compound D) are observed.

TABLE 5
Induction of CYP1A mRNA In HepG2 cells.

| | CYP1A1 mRNA |
|---|---|
| | fold change from |
| Compound | vehicle |
| E | 2295 |
| D | 596 |
| A | 266 |
| B | 84 |
| F | 436 |
| G | 804 |
| C | 19 |
| K | 391 |
| L | 558 |
| M | 457 |
| N | 751 |
| O | 245 |
| P | 388 |

### Example 9

### Induction of CYP1A1 in HEP G2 cells as measured by the cleavage of 7-ethoxyresorufin to resorufin (EROD assay)

The essential methodology for culture and handling of HEP G2 cells and NI-11(cells has been described in Examples 2, 3 and 8. HepG2 cells are seeded at between 1×10⁴ and 2.5×10⁴ cells/well in a 96-well microtitre plate. After 3 days the original growth medium is replaced with 200 μl medium per well containing vehicle (typically 1% ethanol) or test agent in 1% ethanol then incubated for 24 h. ˜95% of the medium is removed and replaced with 200 μl of phenol-red free medium containing 8 μM 7-ethoxyresorufin and 10 μM dicoumarol and incubated for 1 h at 37° C. 100 μl of medium is then transferred to a fresh black 96-well plate and 130 μl methanol added. The formation of resorufin is quantified by fluorometry with a fluorescence plate reader. Fluorescence is determined at λₑₓ 570 nm and λₑₘ 590 nm. The amount of resorufin is calculated from a standard curve generated using 0 to 1 μM resorufin in water at ½ log dilutions. The remaining (i.e. all cells present at the beginning of analysis, since only supernatant is taken to assay EROD) live cells are subsequently utilized for the MIT cytotoxicity assay. MTT is made up in serum-free medium at 5 mg/ml. then added to the cells giving a final concentration of 10% (0.5 mg/ml MTT final) then incubated for 2 h. The medium is carefully removed, then MTT formazan is solubilized by adding 200 μl of DMSO and subsequently mixed on a plate shaker. The absorbance is measured spectra photometrically at 550 nm and background absorbance at 690 nm is subtracted. A standard curve generated using MIT formazan (dissolved in DMSO) from O—200 μM (0, 2, 5, 10, 20, 50, 100, 200 μM) is used to calculate the final experimental values which are then expressed as (wnol resarufin/mol MTT formazan) or as fold-Induction compared to solvent.

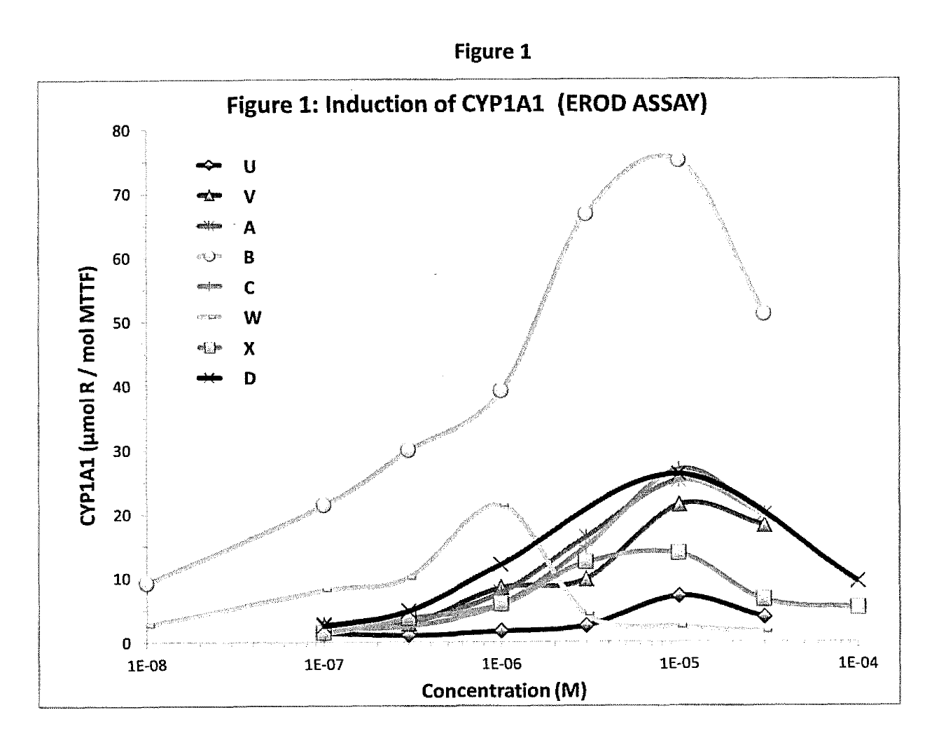

The results are shown in Table 6 below and in FIG. 1. In this assay Compound B and other compounds are potent inducers of the CYP1A1 enzyme in HepG2 cells at 10 uM. When these results are combined with other In vitro and In viva results, AhR agonists with a different (Improved) profile compared to 3-Phenyl-1H-benzo(fIchromen-1-one (compound D) are observed.

TABLE 6
Induction of CYP1A enzyme In HepG2 cells as measured
by cleavage of the CYP1A1 substrate, Ethoxyresorufin
(Ethoxyresorufin-O-deethylase or EROD assay).

| | % change vs. | | Toxicity |
|---|---|---|---|
| | TCDD EROD | EROD EC₅₀ | threshold |
| Compound | (HepG2) at 10 uM | (μM) | (μM) |
| E | 53 | 0.08 | >10 |
| D | 19 | 1 | >50 |
| A | 27 | 2 | >30 |
| B | 88 | 1 | >30 |
| F | 39 | 2 | >30 |
| G | 85 | 2 | 100 |
| H | 77 | 1.5 | >100 |
| I | 16 | 5 | >100 |
| J | 48 | 1.5 | 100 |
| C | 29 | 2.5 | >30 |
| K | 20 | 0.5 | >100 |
| L | 26 | 0.6 | >100 |
| M | 9 | 3 | >30 |
| N | 13 | 1.5 | >30 |
| O | 11 | 5 | >100 |
| P | 45 | 2 | >100 |

### Example 10

### Induction of CYP1A1 in Human Cultured Sebocytes as Measured by the Cleavage of 7-ethoxyresorufin to Resorufin (EROD Assay)

The essential methodology for culture and handling of human cultured sebocytes has been described in in earlier examples for HEPG2 and NHK cells. Cells are seeded at between 1×10⁴ and 2.5×10⁴ cells/well in a 96-well microtitre plate. After 3 days the original growth medium is replaced with 200 medium per well containing vehicle (typically 1% ethanol) or test agent in 1% ethanol then incubated for 24 h. ˜95% of the medium is removed and replaced with 200 μl of phenol-red free medium containing 8 μM 7-ethoxyresorufin and 10 μM dicoumarol and incubated for 1 h at 37° C. 100 μl of medium is then transferred to a fresh black 96-well plate and 130 μl methanol added. The formation of resorufin is quantified by fluorometry with a fluorescence plate reader. Fluorescence is determined at λₑₓ 570 nm and λₑₘ 590 nm. The amount of resorufin is calculated from a standard curve generated using 0 to 1 μM resorufin in water at ½ log dilutions. The remaining (i.e. all cells present at the beginning of analysis, since only supernatant is taken to assay EROD) live cells are subsequently utilized for the MU cytotoxicity assay. MU is made up in serum-free medium at 5 mg/mi. then added to the cells giving a final concentration of 10% (0.5 mg/ml MIT final) then incubated for 2 h. The medium is carefully removed, then MIT formazan is solubilized by adding 200 μl of DMSO and subsequently mixed on a plate shaker. The absorbance is measured spectrophotometrically at 550 nm and background absorbance at 590 nm is subtracted. A standard curve generated using MTT formazan (dissolved in DMSO) from O—200 μM (0, 2, 5, 10, 20, 50, 100, 200 μM) is used to calculate the final experimental values which are then expressed as (μmol resorufin/mol MTT formazan) or as fold-induction compared to solvent.

The results are shown in Table 7 below. In this assay several compounds are potent inducers of the CYP1A1 enzyme in human sebocytes at 10 uM. When these results are combined with other in vitro and in vivo results, AhR agonists with a different (improved) profile compared to 3-Phenyl-1H-benzo[f]chromen-1-one (compound D) are observed.

TABLE 7
Induction of CYP1A enzyme in human sebocyte cells as
measured by cleavage of the CYP1A1 substrate, Ethoxyresorufin
(Ethoxyresorufin-O-deethylase or EROD assay).

| | % change vs. | | Toxicity |
|---|---|---|---|
| | TCDD EROD | EROD EC₅₀ | threshold |
| Compound | (Sebs) at 10 uM | (μM) | (μM) |
| E | 57 | 0.5 | 10 |
| D | 17 | 1 | 30 |
| A | 53 | 2 | 30 |
| B | 57 | 0.05 | >30 |
| F | 13 | 0.2 | 50 |
| G | 93 | 0.25 | 30 |
| H | 73 | 0.25 | >30 |
| I | 1 | NA | >30 |
| J | 39 | 0.05 | >30 |
| C | 23 | 0.2 | >30 |
| K | 19 | 0.5 | >50 |
| L | 18 | 0.5 | >50 |
| M | 1 | NA | 50 |
| N | 1 | NA | >50 |
| O | 1 | NA | >100 |
| P | 34 | 0.2 | >100 |

### Example 11

### Rodent Ear Assay for Determination of Sebum Secretion In Vivo

Rodent ear models (e.g., Luderschmidt (1977)) are validated and represent convenient animal models for testing whether compounds are capable of modulating sebaceous gland function and sebum secretion in vivo. Putative lipid modulators are screened by dosing topically to the ventral surfaces of both the right and left ears BID for 1-4 weeks. At sacrifice, samples of ear tissue are taken for lipid analysis, histology, and skin concentrations of the test compound. Lipid analysis is performed using either HPLC and/or LC/MS. To avoid confusion with epidermal lipids, wax esters, which are a unique product of sebaceous glands, are analyzed as one surrogate of sebum production. Other sebaceous lipids, such as cholesterol esters and triglycerides, are also measured. Histological analysis includes determination of sebaceous gland size and surface area. Good biological activity in these animal models may be the function of increased drug potency, improved skin penetration, improved partitioning into sebum with enhanced access to sebaceous glands, or a variety of other factors.

### Example 12

### Mouse Ear Assay for Determination of Enzyme Expression (Including CYP1A1)

To reproduce the useful effect of 3-phenyl-1H-benzo[f]chromen-1-one, in a therapeutic reduction protocol, activity of test compounds when applied topically to the ears of C57/BL/6 mice is determined. Mice (3-5 animals per group) are treated once a day for 7 days with various compounds according to the invention. Test compounds are solubilized to a final concentration of 1% in acetone or to a lower concentration as noted due to solubility limitations. Each compound in solution is applied to one ear of each mouse in a group. Mice in a separate group received the vehicle as a treatment. After 7 days of treatment with either test compound or vehicle, mice are sacrificed, and the treated ears are recovered and saved in frozen. Pieces of each ear are sectioned and either frozen or placed in formalin for subsequent sectioning. Immunohistochemical techniques (anti-CYP1A1 specific antibody) are used to visualize CYP1A1 expression levels. Levels of the mRNA of sebogenic enzymes are also determined in separate pieces of ear tissue using a PCR technique: after RNA extraction, RTqPCR reaction is performed for mRNA of several major enzymes (fatty desaturase 2 [FADS2], acyl-CoA wax alcohol acyltransferase 1 [AWAT1], as well as elongation of very long chain fatty acids protein 3 [ELOV3] involved in the production of sebaceous lipids. In addition, RTqPCR reaction is optionally performed for mRNA of the enzymes Stearoyl-CoA desaturase-1 [SCD-1] and Acetyl-CoA carboxylase [ACC]. Further samples of ear are submitted to Western blot. Levels of expressed enzymes or mRNA are compared to levels detected in vehicle-treated ears.

### Example 13

### Mouse Ear Assay for Determination of Sebaceous Gland Prevalence

C57BL/6.I male mice (Harlan Models), approximately 4-5 weeks of age, body weight 20-30 g, were acclimatized (individually housed) for 5 days in a 12 hour light/dark cycle standard laboratory environment (T=20-24° C., relative humidity 30-70%, 15-20 air changes per hour) with access to water and standard rodent chow (Harlan Teklad 2014C supplied by Harlan Laboratories Models, S.L.) ad libitum. Animals were anesthetized with isoflurane and the test article (80 ul, 1% solution in DMSO:acetone 50:50) was administered to the outer surface of each of the ears of between 2 and 5 mice twice a day for five days by dermal application using a micropipette tip (a little volume of the solution was also applied on the inner side of the ears). All animals were monitored daily for reaction to the treatment, signs of illness, and behavioral changes. Body weights were recorded before the first administration and immediately prior to sacrifice. Approximately 12 to 16 hours after the final administration on day 5 the mice were sacrificed by i.p. injection of sodium pentobarbital (200 mg/kg) and the ears were collected. Each ear was sectioned into two halves. For each animal, one-half of one ear was fixed, in neutral phosphate-buffered 4% formaldehyde solution (4% formalin). This trimmed section of the ear was then processed, embedded in paraffin wax and stored at room temperature. The remaining half of that ear and the entire opposite ear was frozen at −80° C. Wax mounted ear pieces were sectioned with a microtome and stained with hematoxylin and eosin. Sebaceous glands were identified visually by their structures. Automated tissue imaging analysis was employed to determine the number of active sebaceous glands per ear, the relative surface of the section occupied by sebaceous glands, and/or the number of differentiated and mature sebocytes per square millimeter within the sebaceous glands of a section.

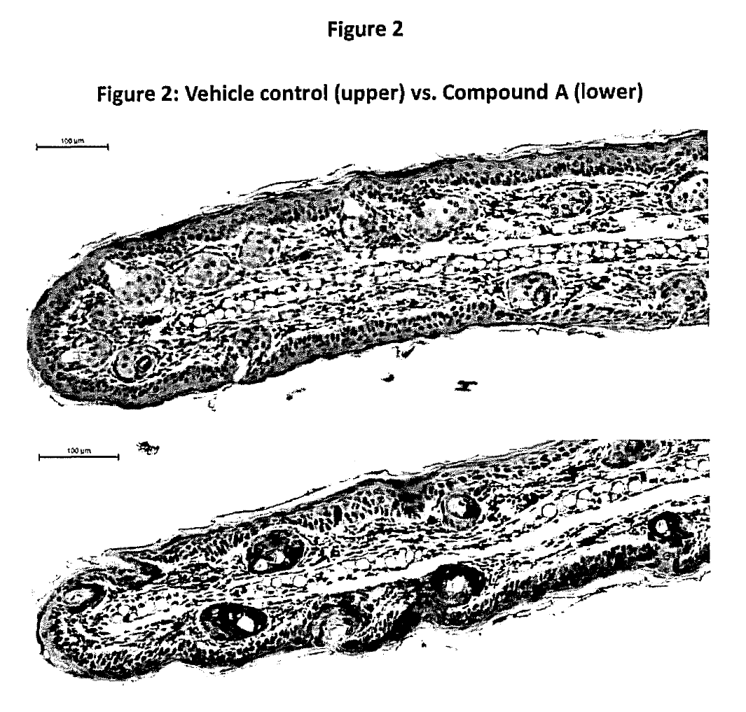

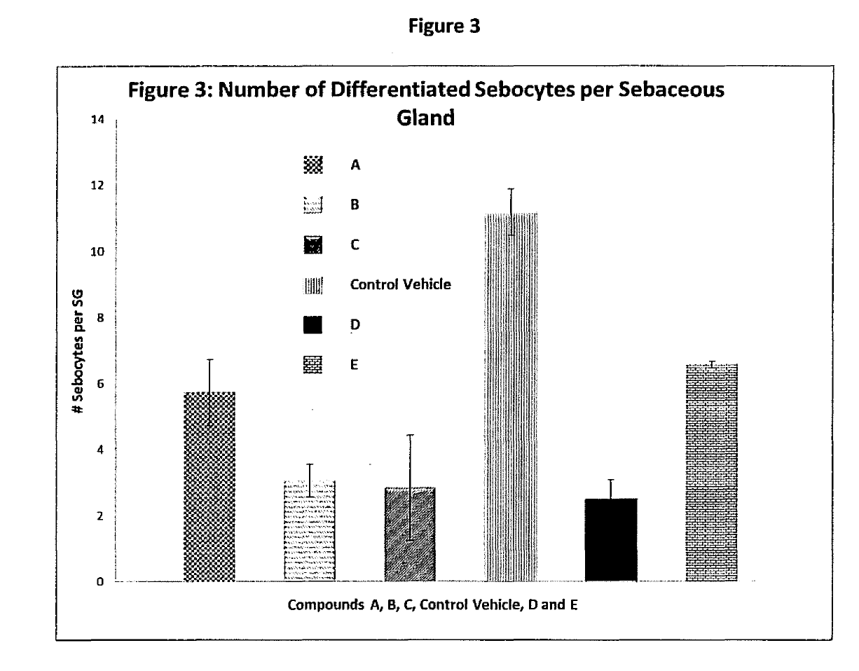

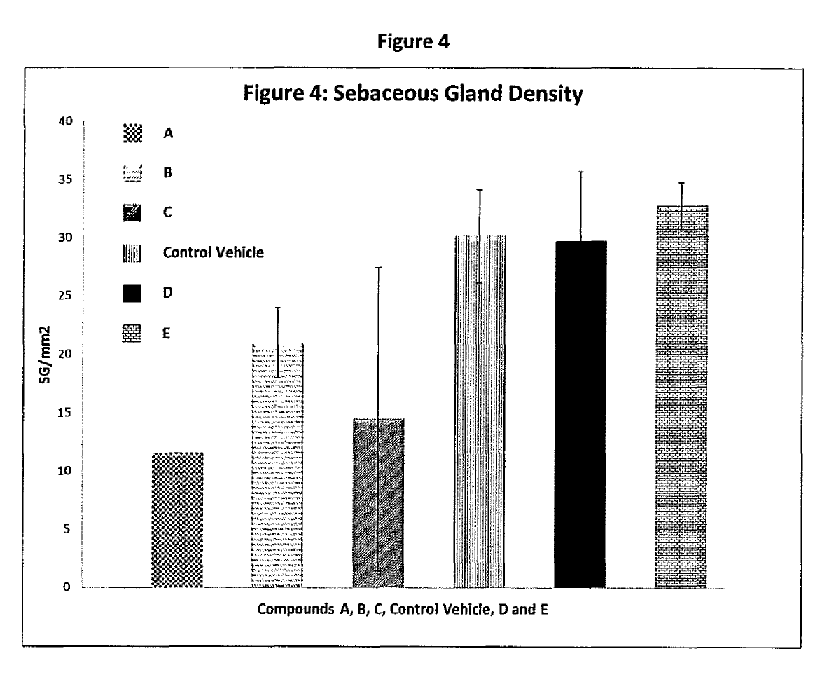

Representative results showing significant activity are shown in FIGS. 2, 3 and 4, which include the results obtained when Compound B of Example 1 was used as a test article in the method of Example 13. Quantitation of the histopathology from FIG. 2 shows, in the case of compound B, a clear reduction in the number of sebaceous glands (FIG. 3) as well as a reduction in the number of differentiated sebocytes (FIG. 4), when compared with controls.

### Example 14

### Tolerability and Effect of Test Articles in Man

A stable 0.5% formulation of an active analog is defined.

Formulation: Active analog 0.5g/100 ml in ethanol/PEG 400 (1:1).

Solvents: Ethanol EMSURE® Merck catalog number 1.00983, batch K42754183.

Polyethylene Glycol (PEG) 400, Fluke catalog number 81 170, batch 260154 286 or PEG 400 Aldrich catalog number 202398.

Stability: No degradation products are observed six months after preparation.

Use in man: The formulation is applied once per day to the face in several patients suffering from intense seborrhea and not eligible for oral treatment with Isotretinoin, some with acne, some with rosacea and one with seborrheic dermatitis.

No side effects are noted. In particular no clinical signs suggesting the onset of microcysts. This provides confirmation in humans of the safety of topical analogs described herein. This tolerability in man therefore amounts to original data of primary importance.

The use of Sebutape® (CUDerm) patch test, to determine the amount of sebum produced in six individuals after treatment, indicated a level corresponding to the normal sebum production range.

### LIST OF REFERENCES

1. PCT International Application Publication No. WO/2013/171696

2. PCT International Application Publication No. WO/2009/093207

3. A. L. Zaenglein et al. (2012). “Acne vulgaris and acneiform eruptions” Fitzpatrick's Dermatology in General Medicine, 8th ed., vol. 1, pp. 897-917

4. J. K. L. Tan (2008) “Current Measures for the Evaluation of Acne Severity” Expert Rev. Dermatol., 3(5), 595-603

5. N. Janiczek-Dolphin et al., (2010) “Can sebum reduction predict acne outcome?” Br. J. Dermatology, 163(4), 683-688

6. K. L. Spalding et al. (2008) “Dynamics of fat cell turnover in humans” Nature, 453, 783-787

7. V. Stella (1975) “Pro-drugs: An Overview and Definition” Pro-drugs as Novel Drug Delivery Systems, T. Higuchi Ed., Vol. 14, Chapter 1, 1-115

8. E. B. Roche (1987) “Bioreversible carriers in drug design: theory and application” Pergamon Press, 1st Edition

9. D. J. Abraham et al. (2010) “Burger's Medicinal Chemistry, Drug Discovery and Development” Wiley, 7th Edition

10. T. L. Lemke et al. (2013) “Foye's Principles of Medicinal Chemistry” Wiley, 7th Edition

11. M. B. Smith (2013) “March's Advanced Organic Chemistry: Reactions, Mechanisms, and Structure” Wiley, 7th Edition

12. S. Hanessian et al. (2013) “Design and Strategy in Organic Synthesis” Wiley-VCH, 1st Edition

13. P. G. M. Wuts et al. “Greene's Protective Groups In Organic Synthesis” Wiley-Interscience, 4th Edition

14. T. L. Ho “Fiesers' Reagents for Organic Synthesis” Wiley, Volume 27 Edition

15. K. Juvale et al. (2013) “Synthesis and biological evaluation of fiavones and benzoflavones as inhibitors of BCRP/ABCG2” Eur. J. Med. Chem. 67: 115-126

16, P. Kulkarni et al. (2012) “An Improved and Eco-Friendly Method for the Synthesis of Flavanone by the Cyclization of 2′-Hydroxy Chalcone using Methane Sulphonic Acid as Catalyst.” Chemistry Journal 02 (03): 106-110 (2012))

17. A. M. Adib et al. (2008) “Synthesis and antimicrobial activity of 4′,5,7-trlhydroxy-3′-prenylflavanone” J. Chem. Sci., 120, (5): 469-473

18. M. A. Selepe et al. (2013) “Application of the Suzuki-Miyaura reaction in the synthesis of flavonoids” Molecules 18(4): 4739-4765

19. Y. Fujita et al. “beta-Naphthoflavone analogs as potent and soluble aryl hydrocarbon receptor agonists: improvement of solubility by disruption of molecular planarity” Bioorg. Med. Chem. 18(3): 1194-203

20. A. Blanco et al. (2003) “A new synthesis of flavonoids via Heck reaction” Tetrahedron Letters, (51) 44: 9107-9109

21. M. Cushman et al. (1991) “Synthesis and protein-tyrosine kinase inhibitory activities of flavonoid analogues” J. Med. Chem. 34(2): 798-806

22. B. A. Bohm (1998) “Introduction to Flavonoids (Chemistry and Biochemistry of Organic Natural Products)” CRC Press; 1st edition, 1-496

23. 1 Liu (2013) “Pyranoflavones: a group of small-molecule probes for exploring the active site cavities of cytochrome P450 enzymes 1A1, 1A2, and 181” J. Med. Chem. 56(10); 4082-4092

24. G. He et al. (2011) “Third-Generation Ah Receptor-Responsive Luciferase Reporter Plasmids: Amplification of Dioxin-Responsive Elements Dramatically Increases CALUX Bioassay Sensitivity and Responsiveness” Toxicological Sci 2011; 123: 511-522

25. P. A. Behnisch (2002) “Screening of Dioxin-like Toxicity Equivalents for Various Matrices with Wildtype and Recombinant Rat Hepato ma H4IIE Cells” Toxciological Sci 2002; 69: 129-130

26. A. R. Gennaro “Remington's Pharmaceutical Sciences” Mack Pub. Co, 19⁶ Edition

27. U.S. Pat. No. 5,962,012

28. U.S. Pat. No. 6,261,595

29. U.S. Pat. No. 6,133,326

30. E. Camera et al. (2010) “Comprehensive analysis of the major lipid classes in sebum by rapid resolution high-performance liquid chromatography and electrospray mass spectrometry” J. Lipid Res., 51(11), 3377-3388

31. C. Luderschmidt et al. (1977) “Effects of cyproterone acetate and carboxylic acid derivatives on the sebaceous glands of the Syrian hamster” Arch. Dermatol. Res., 258(2), 185-191

What is claimed is:

1. A method of treating a skin condition associated with abnormal sebum secretion or abnormal sebaceous gland function in a subject which comprises topically and periodically applying to an area of subject's skin affected by the skin condition a composition comprising a pharmaceutically acceptable carrier and an amount of a compound or of a pharmaceutically acceptable salt of the compound effective to treat the skin condition, wherein the compound has the structure: 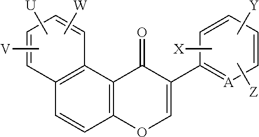
wherein:
each of U, V, W, X, Y, and Z is independently:
H; OH; F; Cl; Br; I; C₁ to C₆ straight chain or branched chain alkyl; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₂)—R; SO₃H or an ester thereof; CO₂H or an ester thereof; PO₃H₂ or a phosphate thereof; PO₂(OCH₃)H or a phosphonate thereof; NO₂; NH₂; NHCH(O); NRCH(O); NHC(O)R; NRC(O)R₁; C(O)NRR₁; C(NH)NRR₁; C(NH)NROH; C(NH)NRNO2; or C(NR)NR₁C (NR₂)NR₃R₄;
wherein adjacent substituents U, V and W or X, Y and Z may form a saturated or unsaturated 5-membered or 6-membered carbocyclic or heterocyclic ring;
wherein each of R, R₁, R₂, R₃ and R₄ if present is independently:
H; OH; O-Rx; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
wherein Rx, if present, is alkyl, cycloalkyl, alkylcycloalkyl, acyl, ester or thioester;
and wherein A is CH or N.

2. The method of claim 1, wherein adjacent substituents U, V and W and X, Y and Z may form a saturated or unsaturated 5-membered or 6-membered carbocyclic or heterocyclic ring.

3. The method of claim 1., wherein:
each of U, V, W, X, Y, and Z is independently:
H; OH; F; Cl; Br; I; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CN R₂)—R; SO₃H or an ester thereof; CO₂H or an ester thereof; PO₃H₂or a phosphate thereof; PO₂(OCH₃)H or a phosphonate thereof; NH₂; NHCH(O); NRCH(O); NHC(O)R; NRC(O)R₁; C(O)NRR₁; C(NH)NRR₁; C(NH)NROH; or C(NR)NR₁C(NR₂)NR₃R₄;
wherein each of R, 112, R₂, R₃ and R₄ if present is Independently:
H, OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
and wherein A is CH or N.

4. The method of claim 2, wherein:
each of U, V, W, X, Y, and Z is independently:
H; OH; F; Cl; Br; I; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a phosphate thereof; PO₂(OCH₃)H or a phosphonate thereof; NH₂; NHCH(O); NRCH(O); NHC(O)R; NRC(O)R₁; C(O)NRR₁; C(NH)NRR₁; C(NH)NROH; or C(NR)NR₁C(NR₂)NR₃R₄;
wherein each of R, R₁, R₂, R₃ and R₄ if present is independently:
H, OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
and wherein A is CH or N.

5. The method of claim 3, wherein:
each of U, V, W, X, Y, and Z is independently:
H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;
wherein each of R and R₁ if present is independently:
H; OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
and wherein A is CH or N.

6. The method of claim 4, wherein:
each of U, V, W, X, Y, and Z is independently:
H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;
wherein each of R and R₁ if present is independently:
H; OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
and wherein A is CH or N.

7. The method of claim 5, wherein:
each of U, V, W, X, Y, and Z is independently:
H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR:
wherein each of R and R₁ if present is independently:
H; OH; optionally substituted alkyl; cycloalkyl; alkylcycloalkyl; heterocycloalkyl; alkylheterocycloalkyl; optionally substituted alkenyl; or optionally substituted alkynyl;
and wherein A is CH or N.

8. The method of any one of claim 1-7, wherein at least one of U, V and W is H and at least one of X, Y and Z is H.

9. The method of any one of claims 1, 3, 5 and 7, wherein at least two of U, V and W is H or at least two of X, Y and Z is H.

10. The method of claim 9, wherein each of U, V and W is H or each of X, Y and Z is H.

11. The method of claim 10, wherein one of U, V and W Is H and each of X, Y and Z is H.

12. The method of claim 10, wherein one of X, Y and Z is H and each of U, V and W is H.

13. The method of claim 10, wherein two of U, V and W is H and each of X, Y and Z is H.

14. The method of claim 10, wherein two of X, Y and Z is H and each of U, V and W is H.

15. The method of claim 1-14, wherein at least one of U, V W, X, Y and Z is other than H.

16. The method of any one of claims 1-10, where the compound is one of the following: 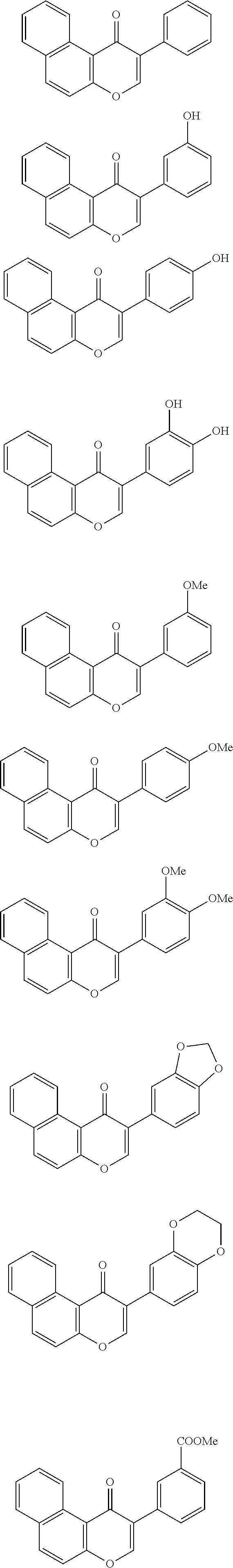 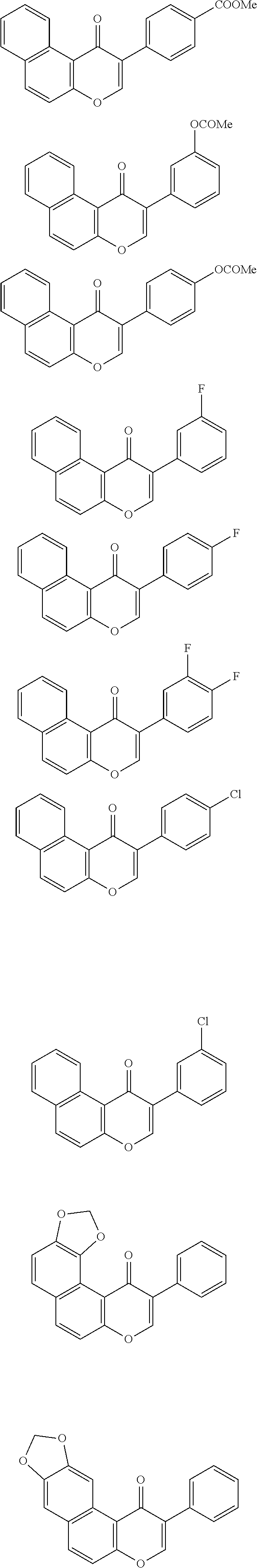 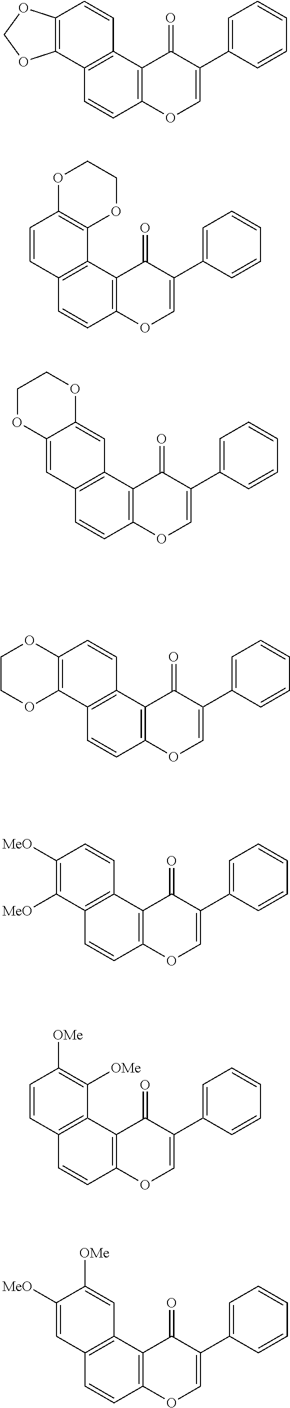 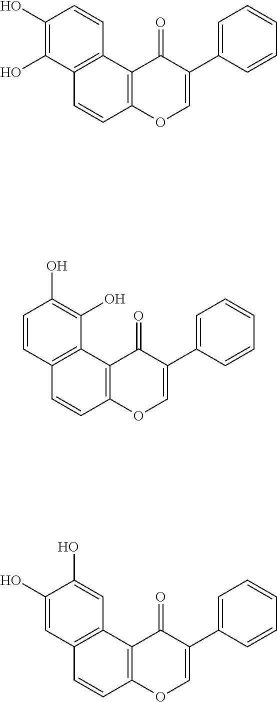 [SMILES: O=c1c(-c2ccccc2)coc2ccc3c(O)c(O)ccc3c12 ; O=c1c(-c2ccccc2)coc2ccc3ccc(O)c(O)c3c12 ; O=c1c(-c2ccccc2)coc2ccc3cc(O)c(O)cc3c12]
or a pharmaceutically acceptable salt, ester or prodrug form thereof.

17. The method of any one of claims 1-10, wherein the compound is one of the following: 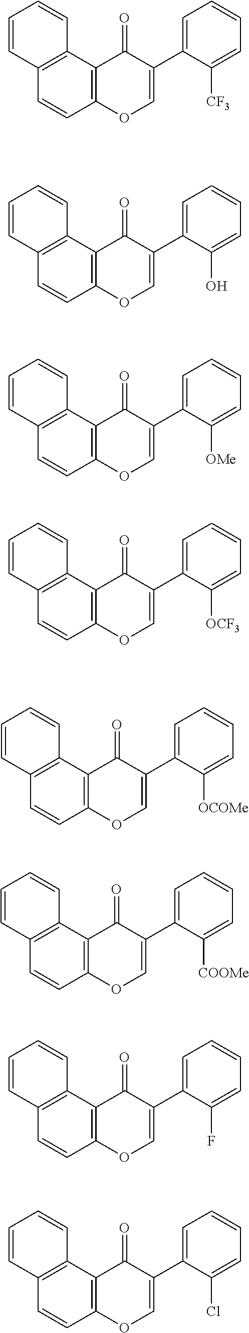 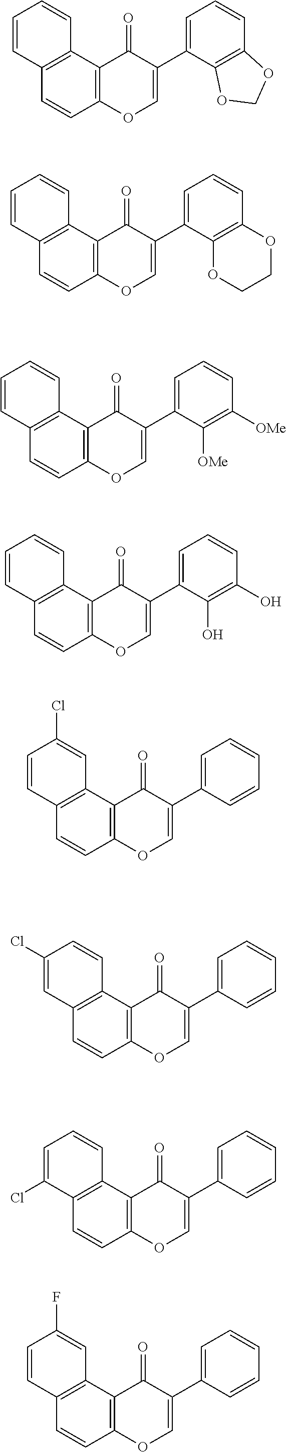 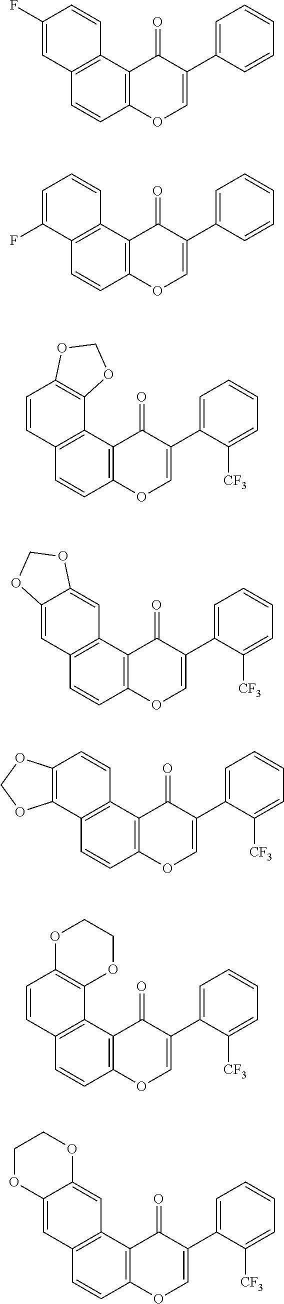 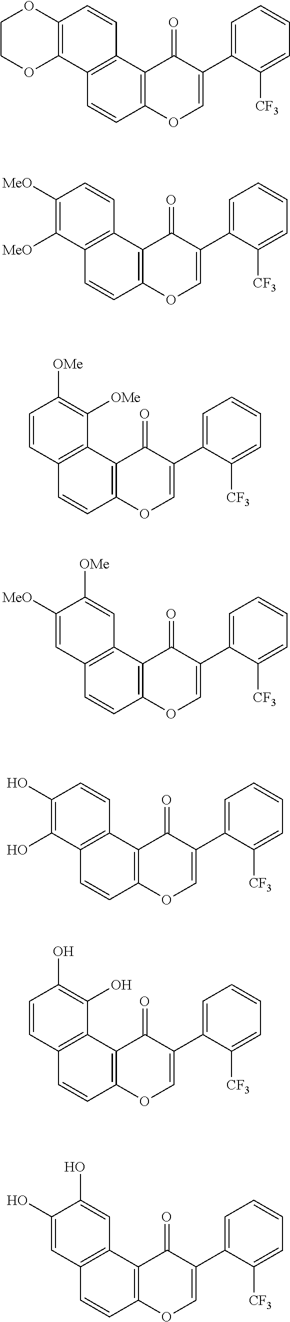
or a pharmaceutically acceptable salt, ester or prodrug form thereof.

18. The method of any one of claims 1-10, wherein the compound is one of the following: 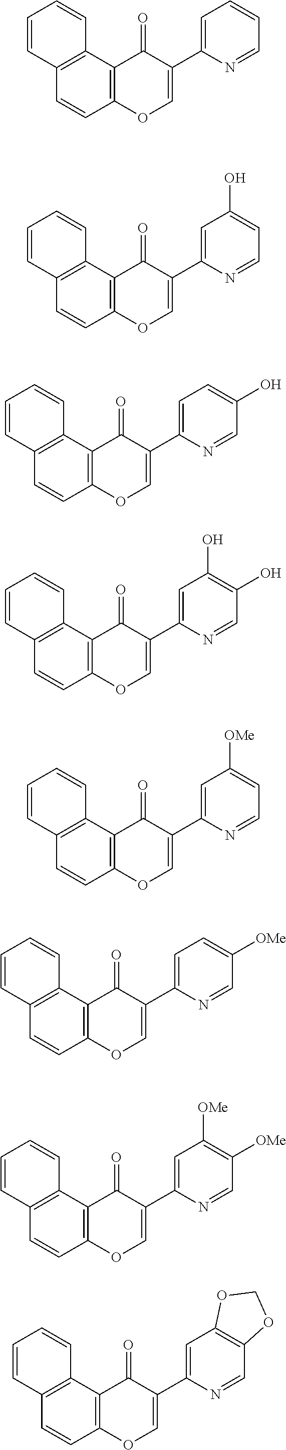 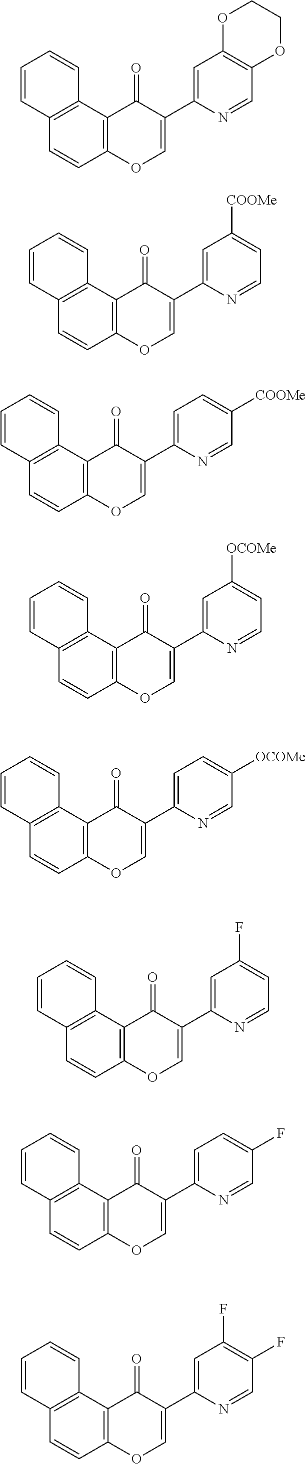 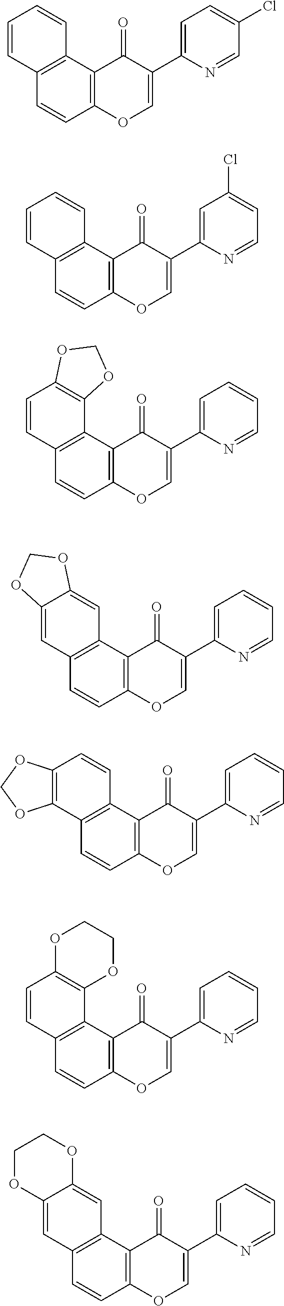 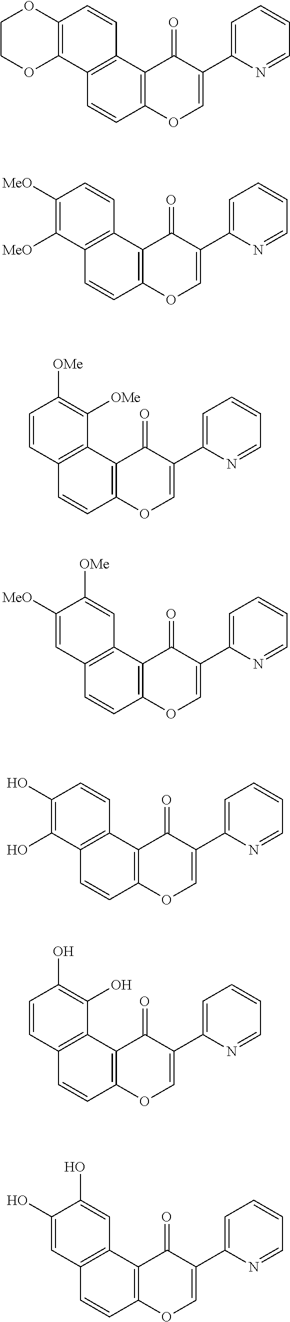
or a pharmaceutically acceptable salt, ester or prodrug form thereof.

19. The method of any one of claims 1-10, wherein the compound is one of the following: 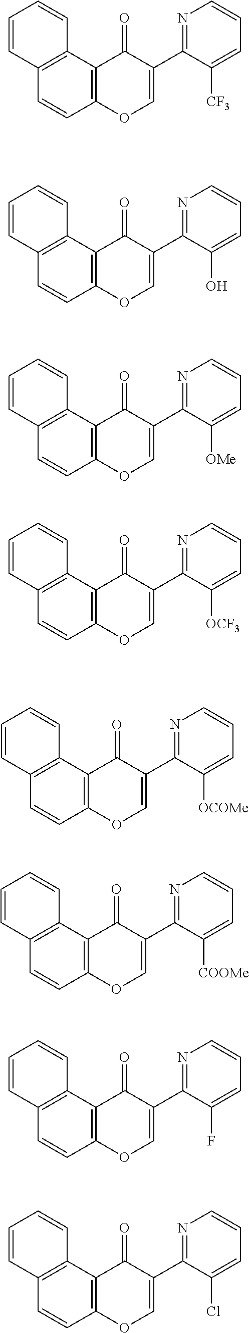 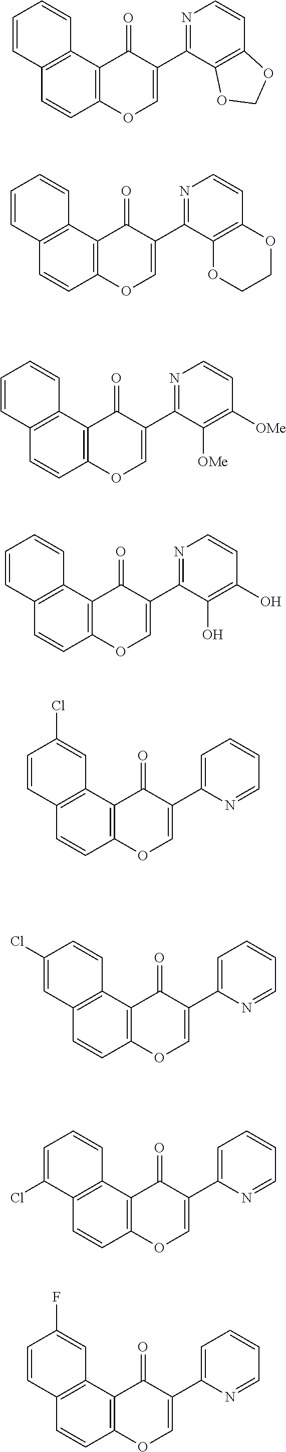 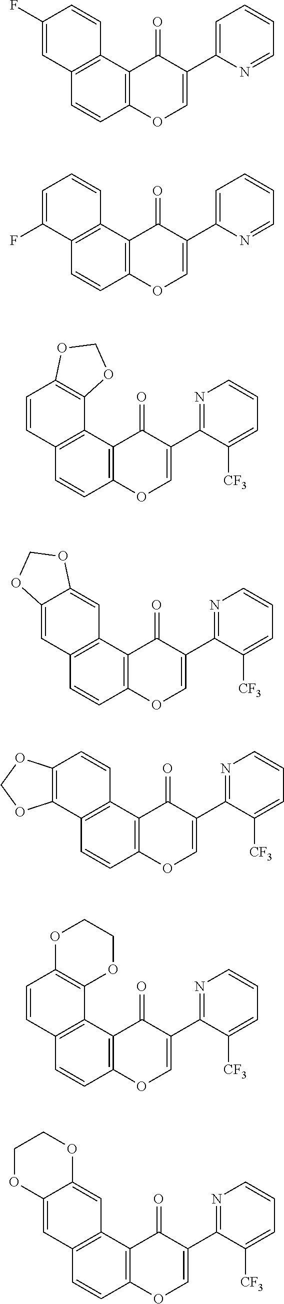 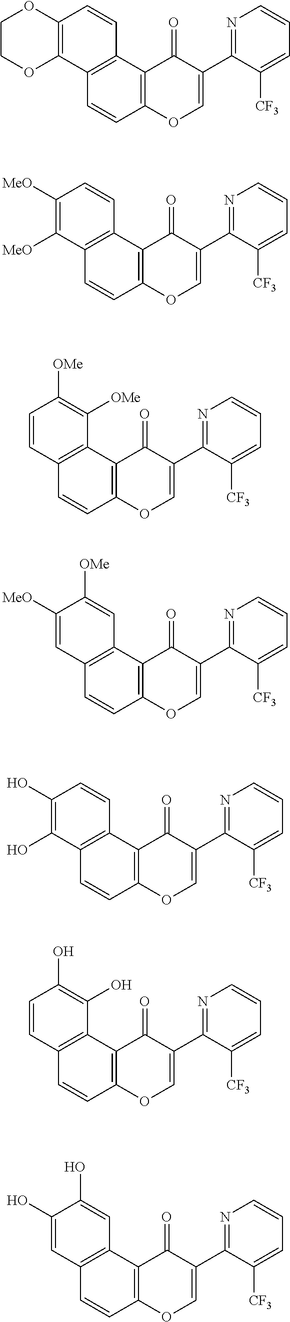
or a pharmaceutically acceptable salt, ester or prodrug form thereof.

20. The method of any one of claims 1-15, wherein an asymmetric center is present in the compound, and the compound is a racemic mixture, a diastereoisomeric mixture, a single enantiomer, an enantiomeric diastereomer, a meso compound, a pure epimer, or a mixture of epimers thereof.

21. The method of any one of claims 1-15 and 20, wherein one or more double bonds present In the compound are cis or trans, E or Z, a cis/trans mixture, an E/Z mixture, a combination of E and Z geometries, a combination of E and Z geometric mixtures or other geometric isomers thereof.

22. The method of any one of claims 1-21, wherein the compound has a lipophilicity as measured by LogP greater than 3.

23. The method of any one of claims 1-22, wherein the pharmaceutically acceptable carrier is suitable for topical use.

24. The method of any one of claims 1-23, wherein the compound has at least one of the following properties:
a) an ability to activate the AhR receptor,
b) an ability to modulate a gene regulated by AhR,
c) an ability to down regulate the expression of genes involved in the synthesis of lipids in sebum,
d) an ability to modulate one or several enzymes involved in lipid metabolism,
e) a short half-life in the human organism of between 0 hours and 96 h, and
f) a measurable positive effect on a recognized criterion of sebaceous hyperactivity.

25. The method of any one of claims 1-24, wherein the skin condition is oily skin, oily hair, shiny or greasy-looking skin, hyperseborrhea, acne, seborrheic dermatitis, rosacea, sebaceous hyperplasia or sebaceous carcinoma.

26. The method of claim 25, wherein the skin condition is acne.

27. The method of claim 25, wherein the skin condition is seborrheic dermatitis.

28. The method of claim 25, wherein the skin condition is rosacea.

29. The method of claim 25, wherein the skin condition is hyperseborrhea.

30. The method of claim 25, wherein the skin condition is sebaceous hyperplasia.

31. The method of claim 25, wherein the skin condition is sebaceous carcinoma.

32. The method of any one of claims 1-31, wherein the compound is present in the composition at a concentration of between about 0.005% and about 5% by weight.

33. A method of treating a disease condition in a subject which comprises administering to the subject a composition comprising a pharmaceutically acceptable carrier and an amount of a compound or of a pharmaceutically acceptable salt of the compound effective to treat the disease condition, wherein the compound has the structure: 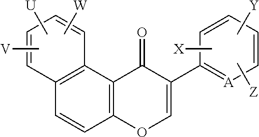
wherein:
each of U, V, W, X, Y, and Z Is independently:
H; OH; F; Cl; Br; I; C₁ to C6 straight chain or branched chain alkyl; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a phosphate thereof; PO₂(OCH₃)H or a phosphonate thereof; NO₂; NH₂; NHCH(O); NRCH(O); NHC(O)R; NRC(O)R₁; C(O)NRR₁; C(NH)NRR₁; C(NH)NROH; C(NH)NRNO2; or C(NR)NR₁C(NR₂)NR₃R₄;
wherein adjacent substituents U, V and W or X, Y and Z may form a saturated or unsaturated 5-membered or 6-membered carbocyclic or heterocyclic ring;
wherein each of R, R₁, R₂, R₃ and R₄ if present is independently:
H; OH; O-Rx; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
wherein Rx, if present, is alkyl, cycloalkyl, alkylcycloalkyl, acyl, ester or thioester;
and wherein A is CH or N;
and wherein the disease condition is pain, inflammation, neurodegenerative diseases, neuropathic pain, trigeminal neuralgia, postherpetic neuralgia, diabetic neuropathy, cancer pain, phantom limb pain, complex regional pain syndrome, and fibromyalgia; rheumatoid arthritis, ankolysing spondylitis, ulcerative colitis, tendonitis, psoriasis, Hidradenitis Suppurativa (sometimes referred to as Acne Inversa) Faber's Disease, Crohn's Disease, rhinitis, skin allergies, asthma, autoimmune diseases with inflammatory components, multiple sclerosis and other demyelinating disorders; Alzheimer's Disease, traumatic brain injury, conditions and diseases characterized by abnormal lipid metabolism and secretion, metabolic disorders, appetite regulation, or obesity.

34. The method of claim 33, wherein the disease condition is psoriasis.

35. A method of treating excess fat in a subject which comprises administering to an area of excess fat a composition comprising a pharmaceutically acceptable carrier and an amount of a compound or of a pharmaceutically acceptable salt of the compound effective to treat the excess fat, wherein the compound has the structure: 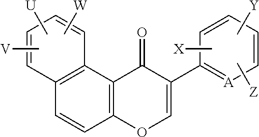
wherein:
each of U, V, W, X, Y, and Z is independently:
H; OH; F; Cl; Br; I; C₁ to C6 straight chain or branched chain alkyl; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a phosphate thereof; PO₂(OCH₃)H or a phosphonate thereof; NO₂; NH₂; NHCH(O); NRCH(O); NHC(O)R; NRC(O)R₁; C(O)NRR₁; C(NH)NRR₁; C(NH)NROH; C(NH)NRNO2; or C(NR)NR₁C(NR₂)NR₃R₄;
wherein adjacent substituents U, V and W or X, Y and Z may form a saturated or unsaturated 5-membered or 6-membered carbocyclic or heterocyclic ring;
wherein each of R, R₁, R2, Ra and R₄ if present is independently:
H; OH; O-Rx; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
wherein Rx, if present, is alkyl, cycloalkyl, alkylcycloalkyl, acyl, ester or thioester;
and wherein A is CH or N.

36. The method of claim 35, wherein the excess fat is an excess of eyelid fat (steatoblepharon, including either or both upper and lower steatoblepharon), otherwise known as eye bags.

37. The method of claim 35, wherein the excess fat is surrounding the eye and is associated with Grave's opthalmopathy.

38. The method of claim 35, wherein the excess fat is a lipoma, a liposarcoma or an excess of submental fat.

39. The method of claim 38, wherein the excess fat is a lipoma.

40. The method of claim 38, wherein the excess fat is a liposarcoma.

41. The method of claim 38, wherein the excess fat is an excess of submental fat.

42. The method of any one of claims 33-41, wherein adjacent substituents U, V and W and X, Y and Z may form a saturated or unsaturated 5-membered or 6-membered carbocyclic or heterocyclic ring.

43. The method of any one of claims 33-41, wherein:
each of U, V, W, X, Y, and Z is independently:
H; OH; F; Cl; Br; I; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a phosphate thereof; PO₂(OCH₃)H or a phosphonate thereof; NH₂; NHCH(O); NRCH(O); NHC(O)R; NRC(O)R₁; C(O)NRR₁; C(NH)NRR₁; C(NH)NROH; or C(NR)NR₁C(NR₂)NR₃R₄;
wherein each of R, R₁, R₂, R₃ and R₄ if present is independently:
H, OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
and wherein A is CH or N.

44. The method of claim 42, wherein:
each of U, V, W, X, Y, and Z Is independently:
H; OH; F; Cl; Br; I; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a phosphate thereof; PO₂(OCH₃)H or a phosphonate thereof; NH₂; NHCH(O); NRCH(O); NHC(O)R; NRC(O)R₁; C(O)NRR₁; C(NH)NRR₁; C(NH)NROH; or C(NR)NR₁C(NR₂)NR₃R₄;
wherein each of R, R₁, R₂, R₃ and R₄ if present is independently:
H, OH; optionally substituted alkyl; cycloalkyl; alkylcycloalkyl; heterocycloalkyl; alkylheterocycloalkyl;
optionally substituted alkenyl; optionally substituted alkynyl; optionally substituted aryl; optionally substituted alkylaryl; optionally substituted heteroaryl; or optionally substituted alkylheteroaryl;
and wherein A is CH or N.

45. The method of claim 43, wherein:
each of U, V, W, X, Y, and Z is independently:
H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;
wherein each of R and R₃ if present is independently:
H; OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
and wherein A is CH or N.

46. The method of claim 44, wherein:
each of U, V, W, X, Y, and Z is independently:
H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;
wherein each of R and R₁ if present Is independently:
H; OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
and wherein A is CH or N.

47. The method of claim 45, wherein:
each of U, V, W, X, Y, and Z is independently:
H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₂)—R; CO₂H or an ester thereof; NH₂; NHCH(O); NRCH(O); NHC(O)R; NRC(O)R₁; C(O)NRR₁;or C(NH)NRR₁;
wherein each of R and R₁ if present is independently:
H; OH; optionally substituted alkyl; cycloalkyl; alkylcycloalkyl; heterocycloalkyl; alkylheterocycloalkyl; optionally substituted alkenyl; or optionally substituted alkynyl;
and wherein A is CH or N.

48. The method of any one of claims 33-47, wherein at least one of U, V and W is H and at least one of X, Y and Z is H.

49. The method of any one of claims 33-41, 43, 45 and 47, wherein at least two of U, V and W is H or at least two of X, V and Z is H.

50. The method of claim 49, wherein each of U, V and W is H or each of X, Y and Z is H.

51. The method of claim 50, wherein one of U, V and W is H and each of X, Y and Z is H.

52. The method of claim 50, wherein one of X, Y and Z is H and each of U, V and W is H.

53. The method of claim 50, wherein two of U, V and W is H and each of X, Y and Z is H.

54. The method of claim 50, wherein two of X, Y and Z is H and each of U, V and W is H.

55. The method of claim 33-50, wherein at least one of U, V W, X, Y and Z is other than H.

56. The method of any one of claims 33-50, where the compound is one of the following: 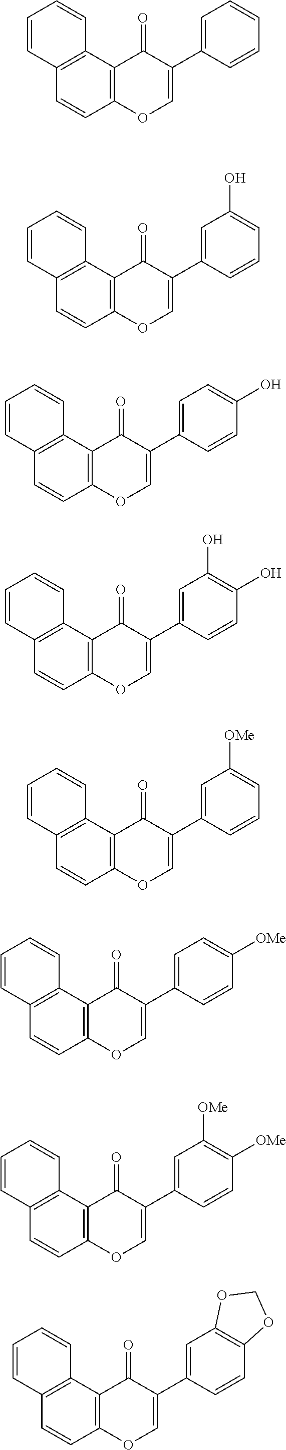 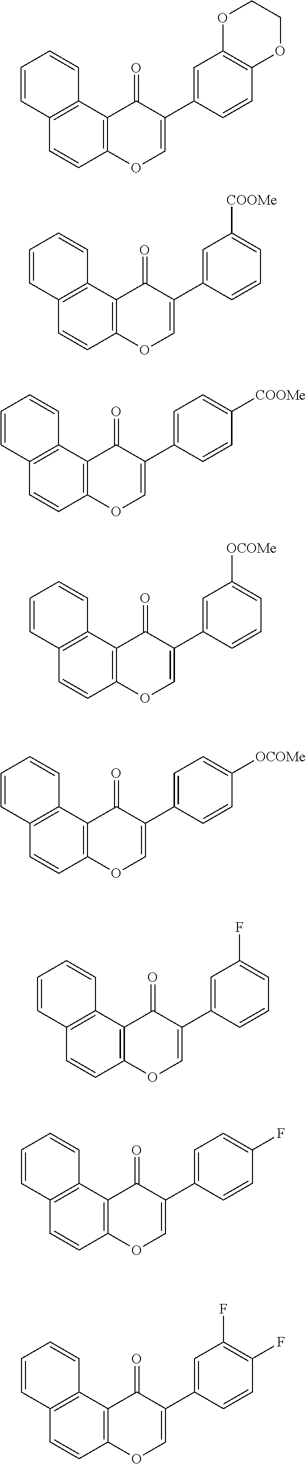 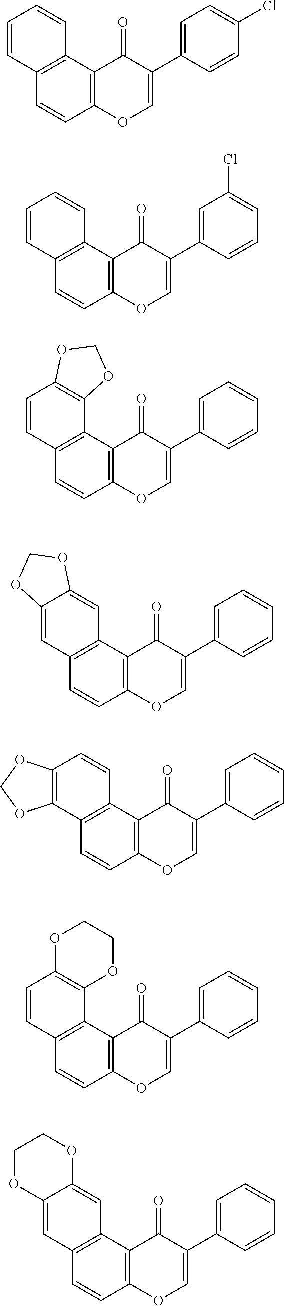 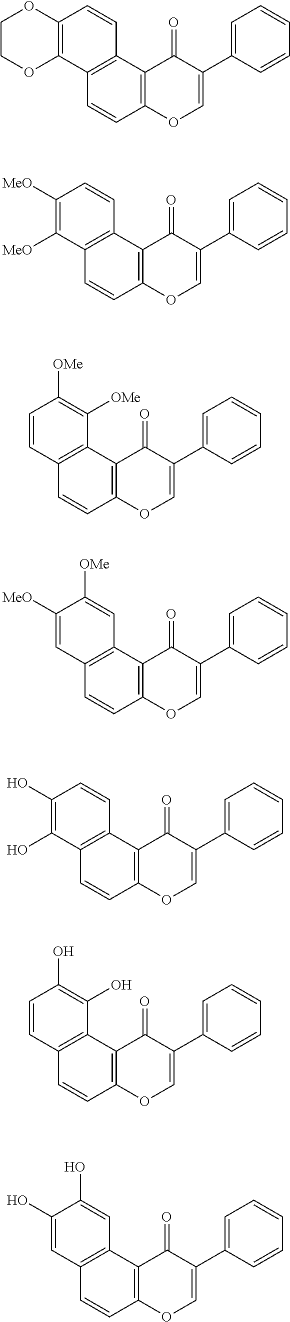
or a pharmaceutically acceptable salt, ester or prodrug form thereof.

57. The method of any one of claims 33-50, wherein the compound is one of the following: 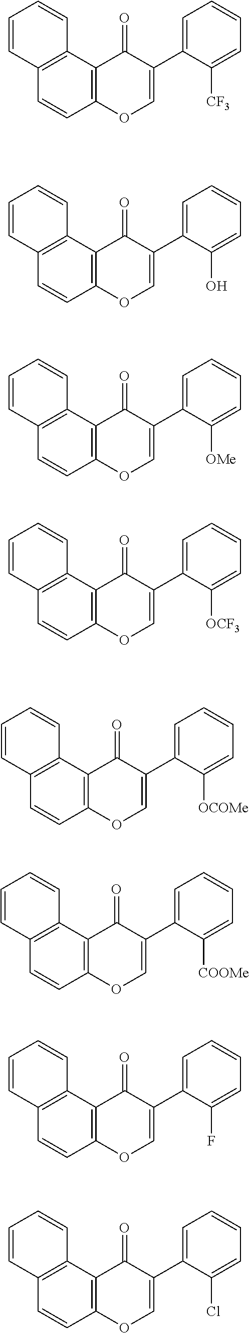 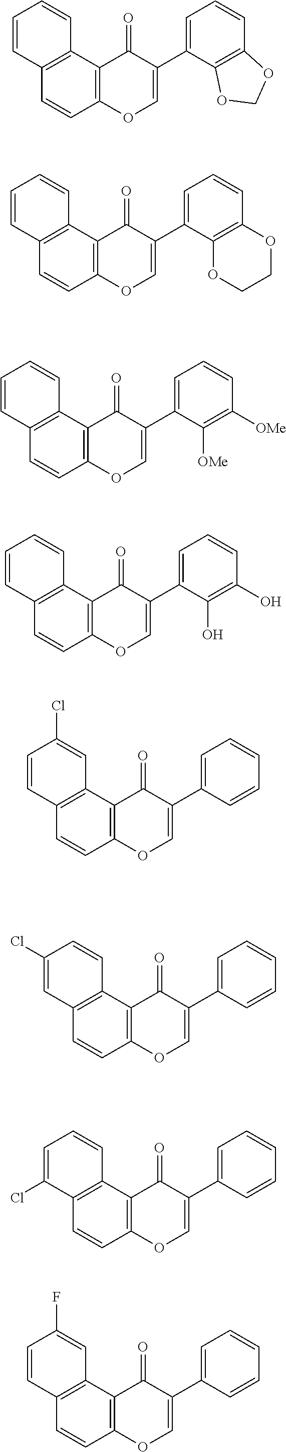 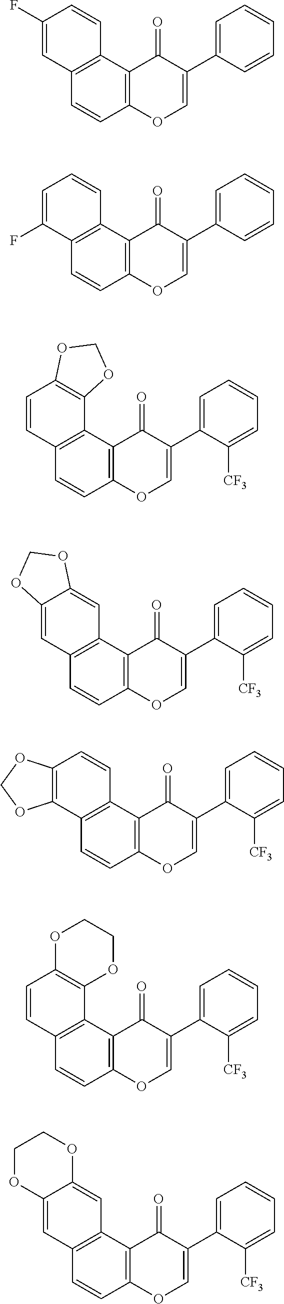 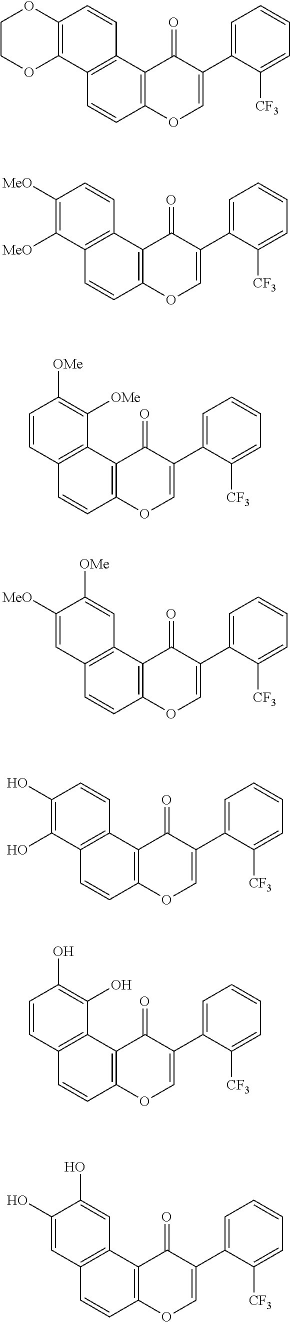
or a pharmaceutically acceptable salt, ester or prodrug form thereof.

58. The method of any one of claims 33-50, wherein the compound is one of the following: 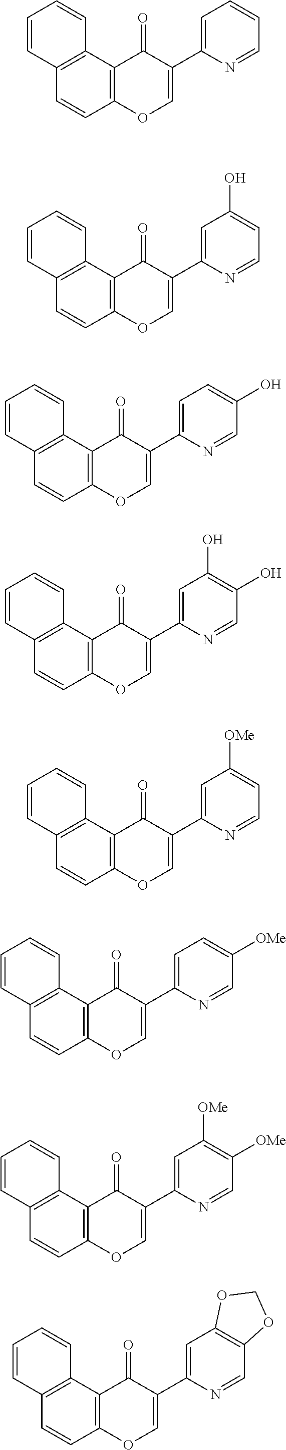 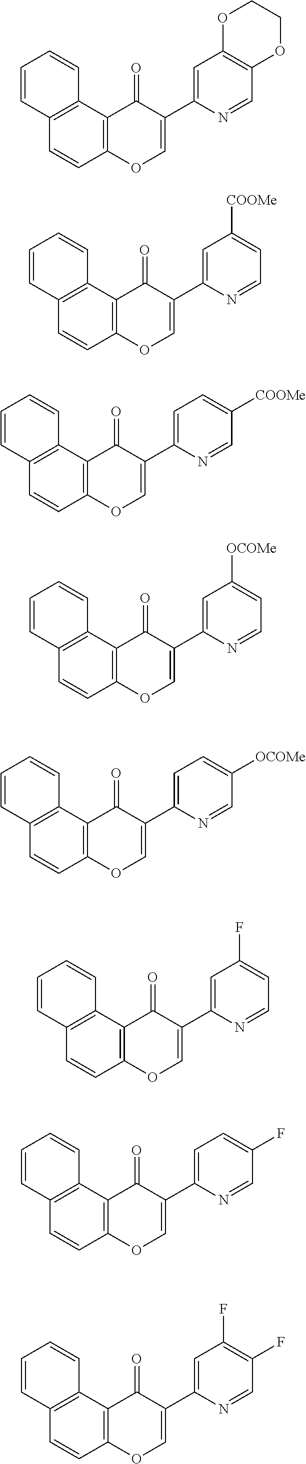 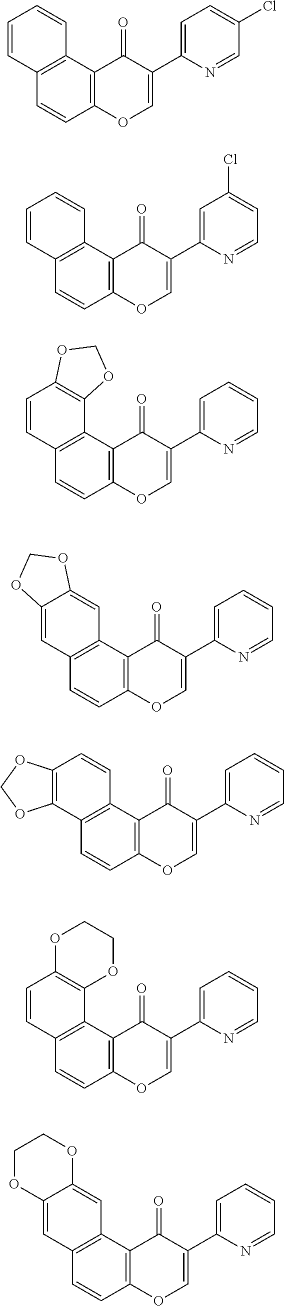 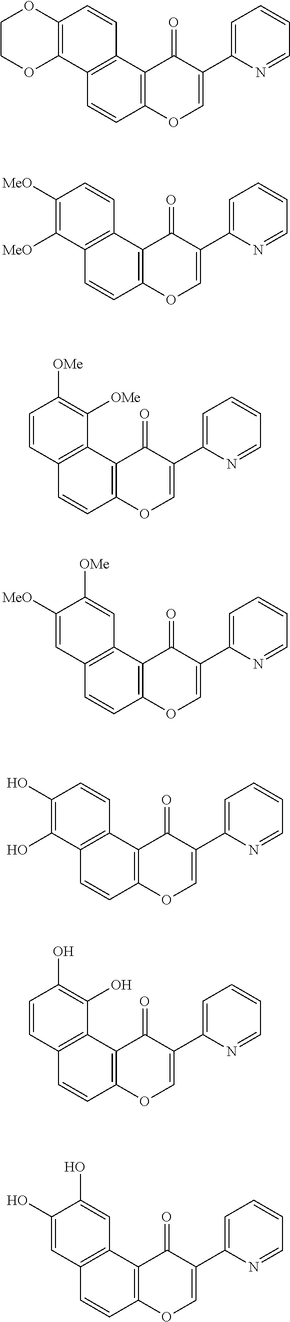
or a pharmaceutically acceptable salt, ester or prodrug form thereof.

59. The method of any one of claims 33-50, wherein the compound is one of the following: 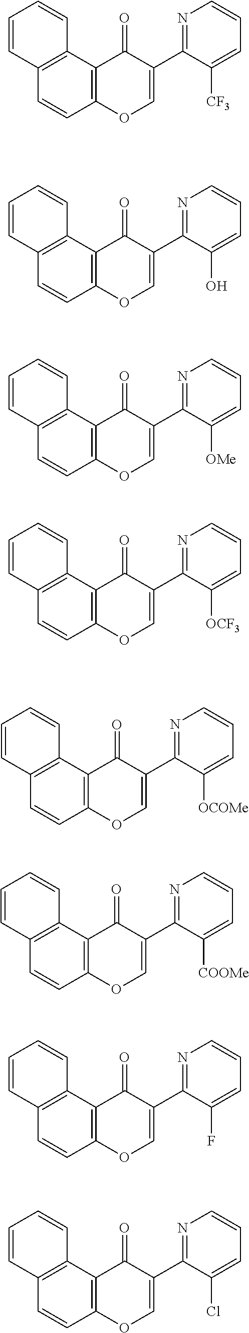 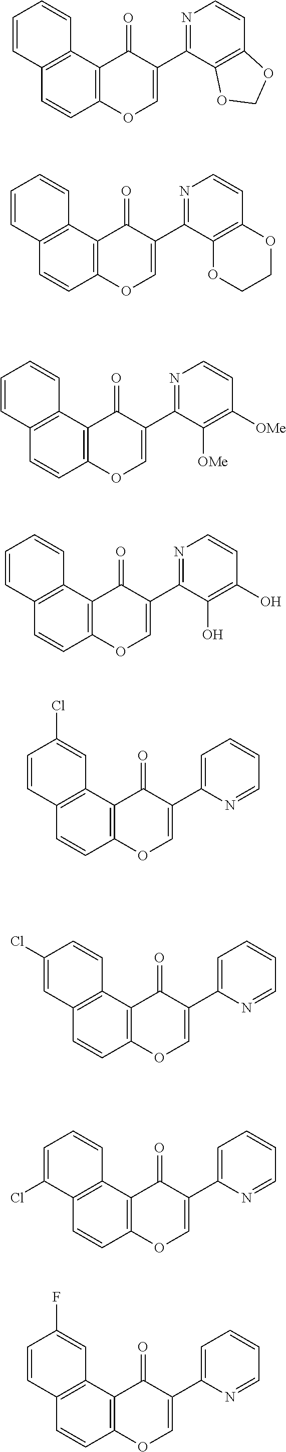 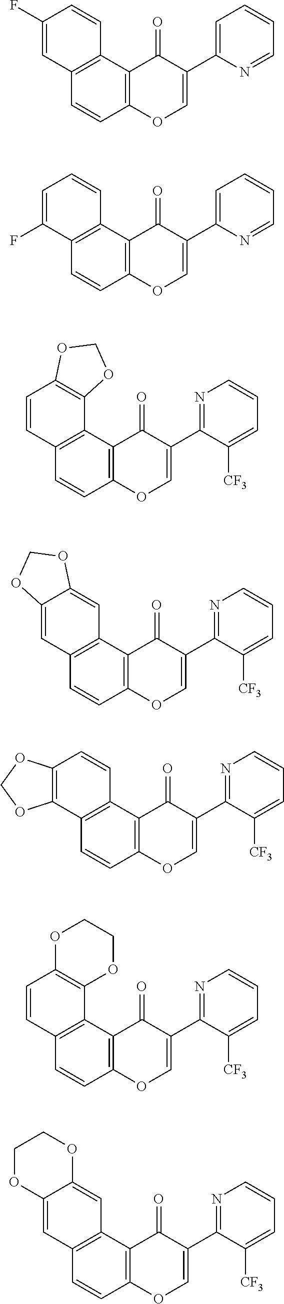 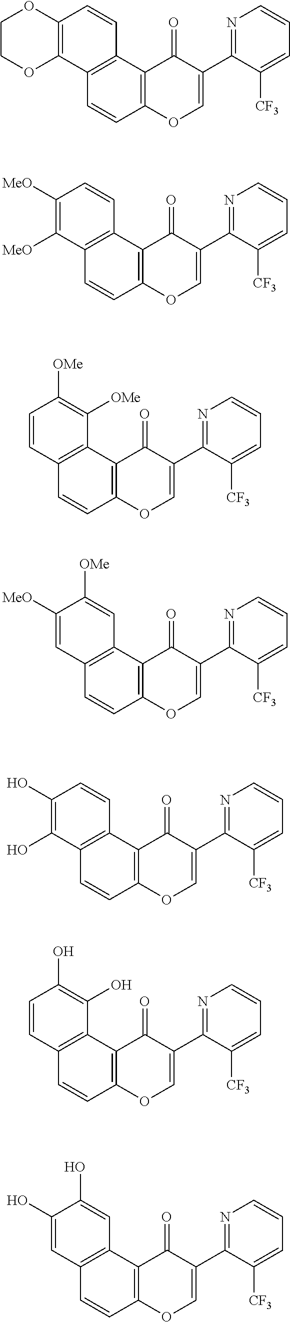
or a pharmaceutically acceptable salt, ester or prodrug form thereof.

60. The method of any one of claims 33-55, wherein an asymmetric center is present in the compound, and the compound is a racemic mixture, a diastereoisomeric mixture, a single enantiomer, an enantiomeric diastereomer, a meso compound, a pure epimer, or a mixture of epimers thereof.

61. The method of any one of claims 33-55 and 60, wherein one or more double bonds present in the compound are cis or trans, E or Z, a cis/trans mixture, an E/Z mixture, a combination of E and Z geometries, a combination of E and Z geometric mixtures or other geometric isomers thereof.

62. The method of any one of claims 33-61, wherein the compound is present in the composition at a concentration between about 0.005% and about 5%.

63. A pharmaceutical composition comprising a pharmaceutically acceptable carrier and a compound or of a pharmaceutically acceptable salt of the compound, wherein the compound has the structure: 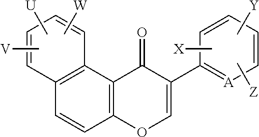
wherein:
each of U, V, W, X, Y, and Z is independently:
H; OH; F; Cl; Br; C₁; C₁ to C6 straight chain or branched chain alkyl; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₂)—R; SO₃H or an ester thereof; CO₂H or an ester thereof; PO₃H₂ or a phosphate thereof; PO₂(OCH₃)H or a phosphonate thereof; NO₂; NH₂; NHCH(O); NRCH(O); NHC(O)R; NRC(O)R₁; C(O)NRR₁; C(NH)NRR₁; C(NH)NROH; C(NH)NRNO2; or C(NR)NR₁C(NR₂)NR₃R₄;
wherein adjacent substituents U, V and W or X, Y and Z may form a saturated or unsaturated 5-membered or 6-membered carbocyclic or heterocyclic ring;
wherein each of R, R₁, R₂, R₃ and R₄ if present is independently:
H; OH; O-Rx; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
wherein Rx, if present, is alkyl, cycloalkyl, alkylcycloalkyl, acyl, ester or thioester;
and wherein A is CH or N.

64. The pharmaceutical composition of claim 63, wherein adjacent substituents U, V and W and X, Y and Z may form a saturated or unsaturated 5-membered or 6-membered carbocyclic or heterocyclic ring.

65. The pharmaceutical composition of claim 63, wherein:
each of U, V, W, X, Y, and Z is independently:
H; OH; F; Cl; Br; I; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₃)—R; SO₃H or an ester thereof; CO₂H or an ester thereof; PO₃H₂ or a phosphate thereof; PO₂(OCH₃)H or a phosphonate thereof; NH₂; NHCH(O); NRCH(O); NHC(O)R; NRC(O)R₁; C(O)NRR₁; C(NH)NRR₁; C(NH)NROH; or C(NR)NR₁C(NR₂)NR₃R₄;
wherein each of R, R₁, R₂, R₃ and R₄ if present is independently:
H, OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
and wherein A is CH or N.

66. The pharmaceutical composition of claim 64, wherein:
each of U, V, W, X, Y, and Z is independently:
H; OH; F; Cl; Br; I; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H2 or a phosphate thereof; PO₂(OCH₃)H or a phosphonate thereof; NH₂; NHCH(O); NRCH(O); NHC(O)R; NRC(O)R₁; C(O)NRR₁; C(NH)NRR₁; C(NH)NROH; or C(NR)NR₁C(NR₂)NR₃R₄;
wherein each of R, R₁, R₂, R₃ and R₄ if present is independently:
H, OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
and wherein A is CH or N.

67. The pharmaceutical composition of claim 64, wherein:
each of U, V, W, X, Y, and Z is independently:
H; OH; F; Cl; CH₃; CH₂F; CHF₂; Ch; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;
wherein each of R and R₁ if present is independently:
H; OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkyl hetero aryl;
and wherein A is CH or N.

68. The pharmaceutical composition of claim 66, wherein:
each of U, V, W, X, Y, and Z is independently:
H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;
wherein each of R and R₁ if present is independently:
H; OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
and wherein A is CH or N.

69. The pharmaceutical composition of claim 67, wherein:
each of U, V, W, X, Y, and Z is independently:
H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;
wherein each of R and R₁ if present is independently:
H; OH; optionally substituted alkyl; cycloalkyl; alkylcycloalkyl; heterocycloalkyl; alkylheterocycloalkyl; optionally substituted alkenyl; or optionally substituted alkynyl; and wherein A is CH or N.

70. The pharmaceutical composition of any one of claim 63-69, wherein at least one of U, V and W is H and at least one of X, Y and Z is H.

71. The pharmaceutical composition of any one of claims 63, 65, 67 and 69, wherein at least two of U, V and W is H or at least two of X, Y and Z is H.

72. The pharmaceutical composition of claim 71, wherein each of U, V and W is H or each of X, Y and Z is H.

73. The pharmaceutical composition of claim 72, wherein one of U, V and W is H and each of X, Y and Z is H.

74. The pharmaceutical composition of claim 72, wherein one of X, Y and Z is H and each of U, V and W is H.

75. The pharmaceutical composition of claim 72, wherein two of U, V and W is H and each of X, Y and Z is H.

76. The pharmaceutical composition of claim 72, wherein two of X, Y and Z is H and each of U, V and W is H.

77. The pharmaceutical composition of claim 63-72, wherein at least one of U, V W, X, Y and Z is other than H.

78. The pharmaceutical composition of any one of claims 63-72, where the compound is one of the following; 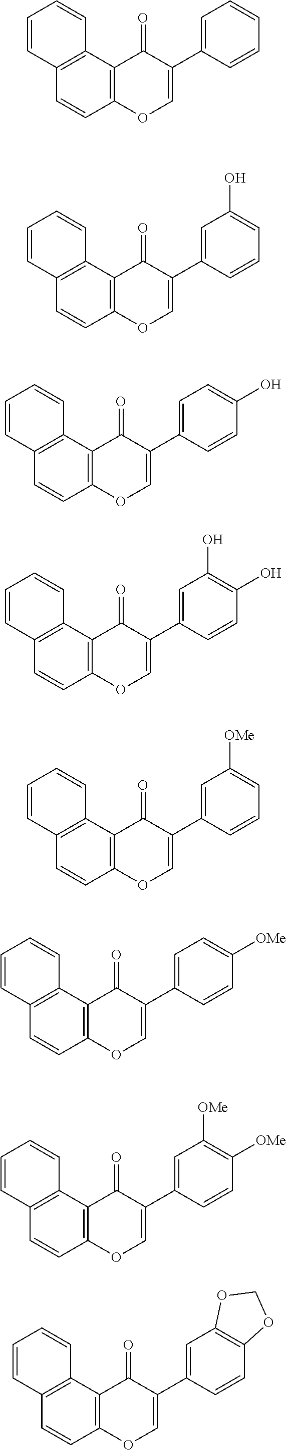 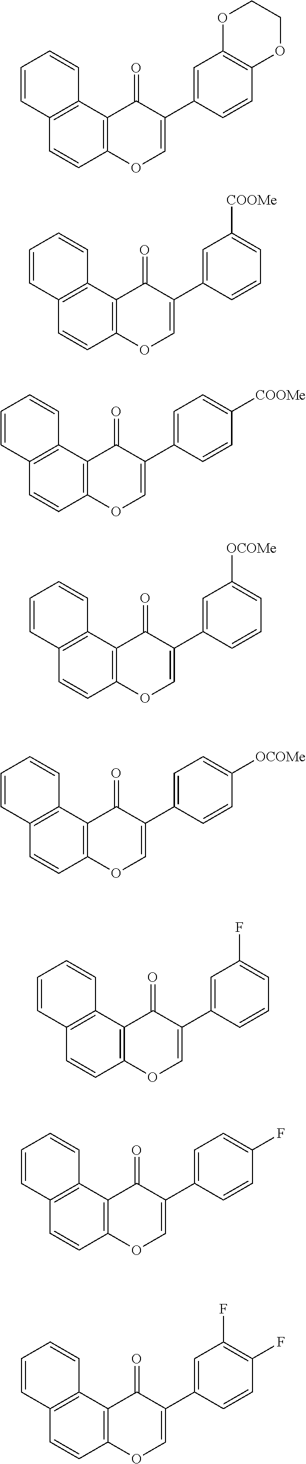 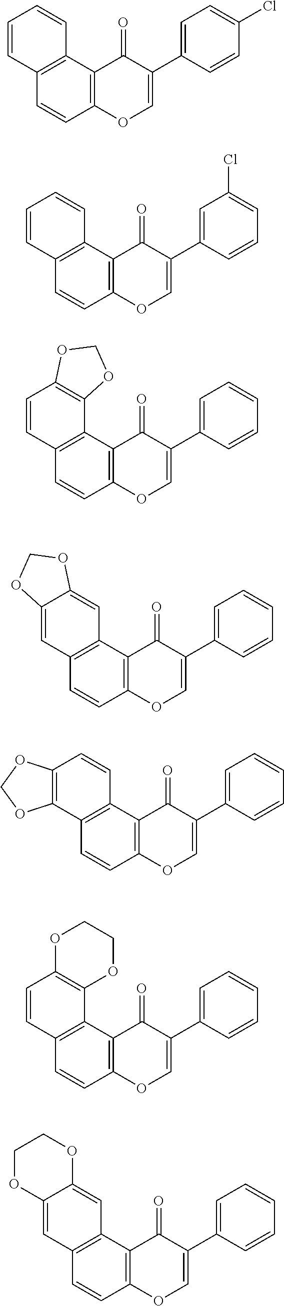 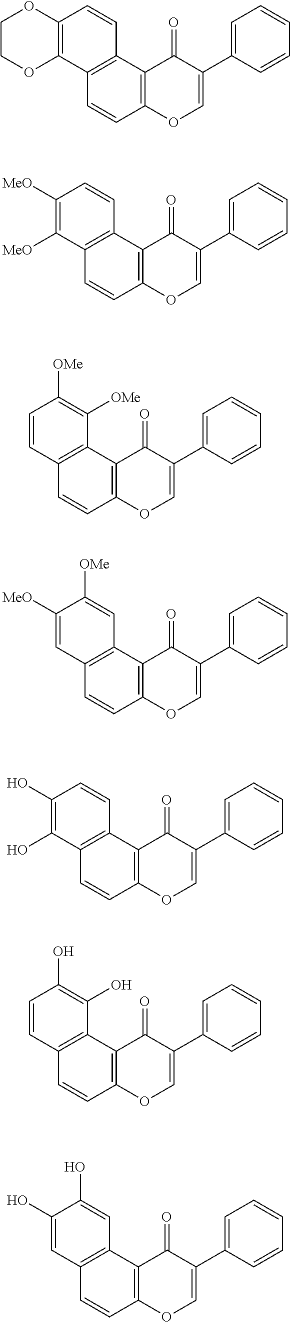
or a pharmaceutically acceptable salt, ester or prodrug form thereof.

79. The pharmaceutical composition of any one of claims 63-72, wherein the compound is one of the following: 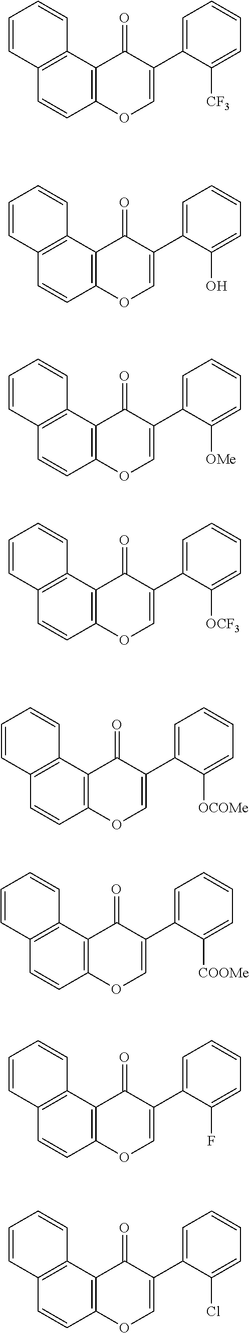 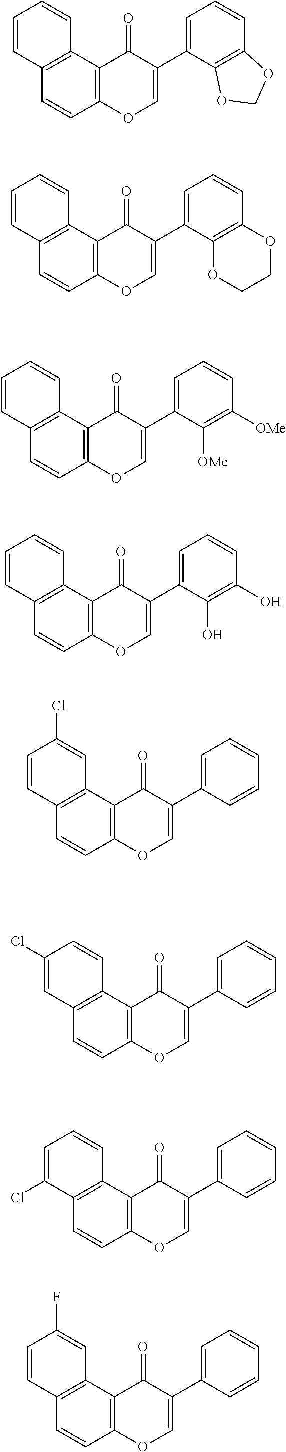 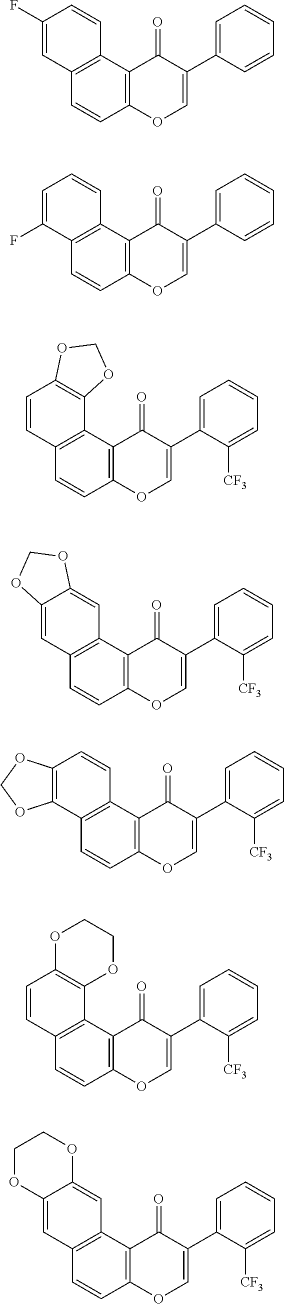 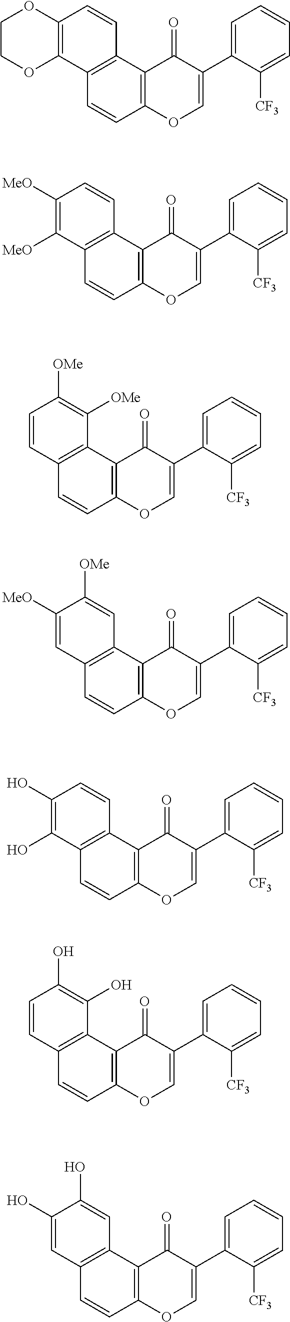
or a pharmaceutically acceptable salt, ester or prodrug form thereof.

80. The pharmaceutical composition of any one of claims 63-72, wherein the compound is one of the following: 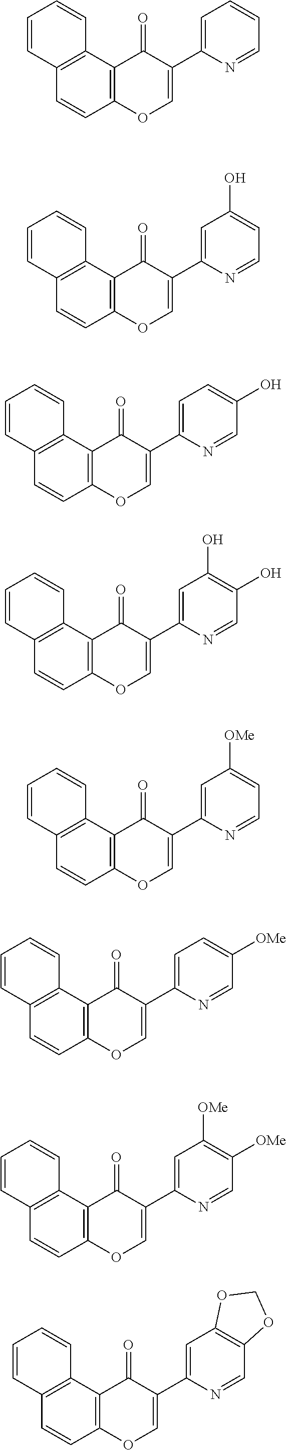 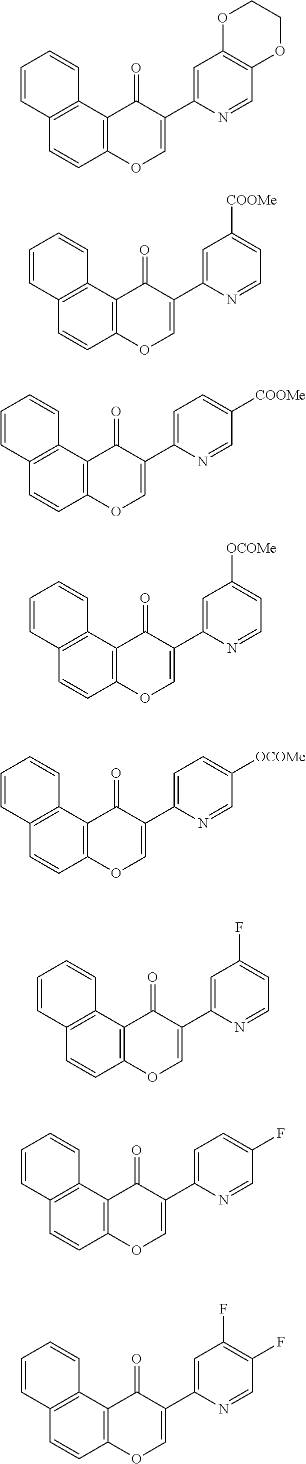 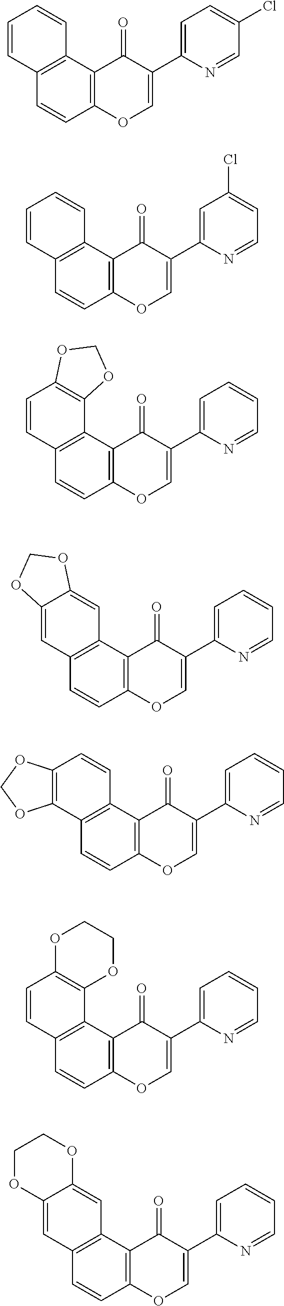 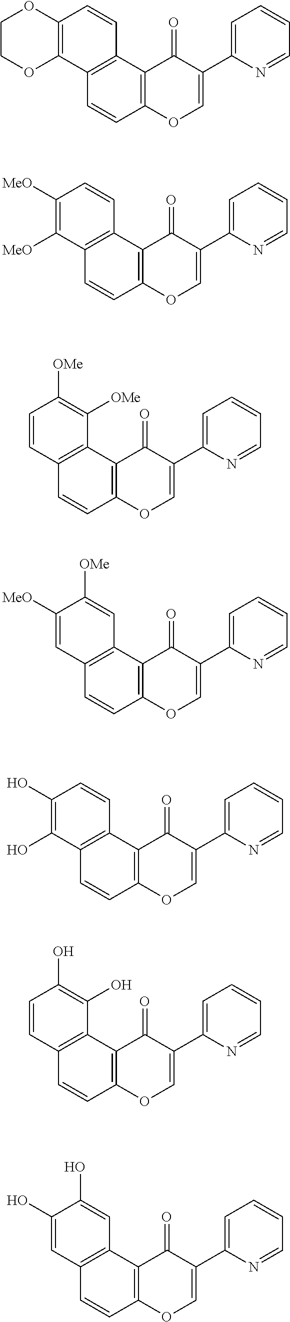
or a pharmaceutically acceptable salt, ester or prodrug form thereof,

81. The pharmaceutical composition of any one of claims 63-72, wherein the compound is one of the following: 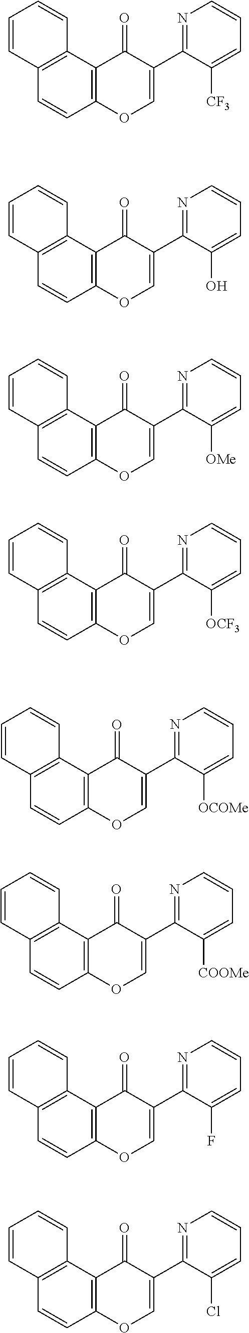 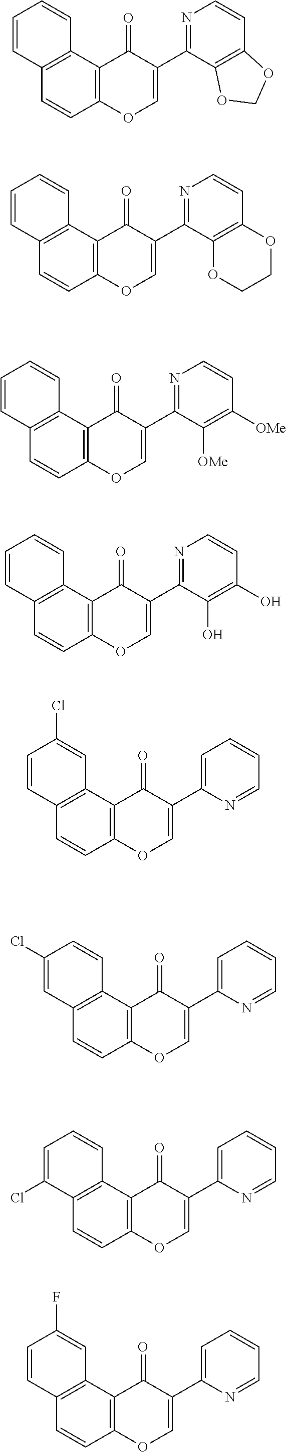 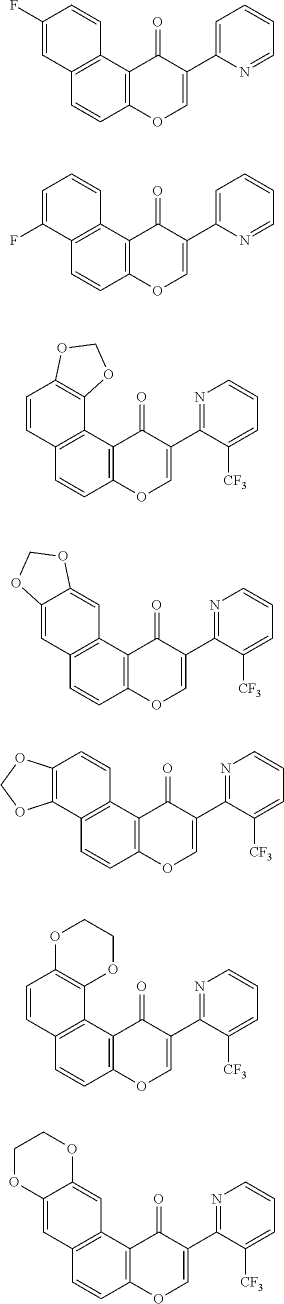 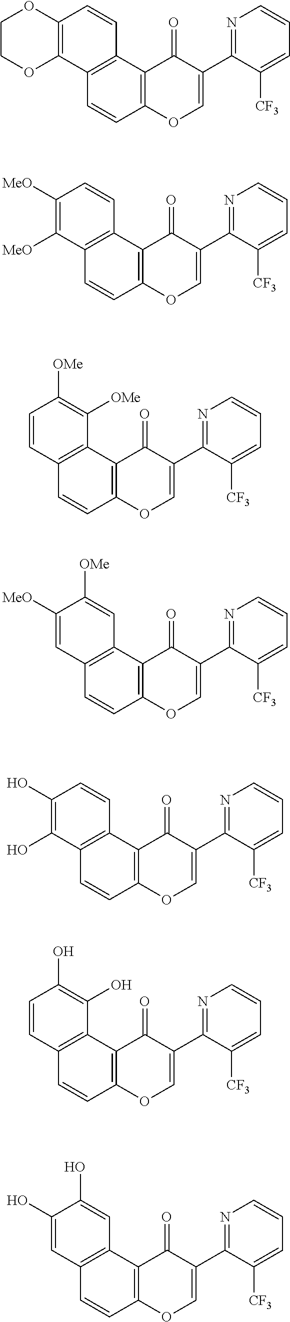
or a pharmaceutically acceptable salt, ester or prodrug form thereof.

82. The pharmaceutical composition of any one of claims 63-77, wherein an asymmetric center is present in the compound, and the compound is a racemic mixture, a diastereoisomeric mixture, a single enantiomer, an enantiomeric diastereomer, a meso compound, a pure epimer, or a mixture of epimers thereof.

83. The pharmaceutical composition of any one of claims 63-77 and 82, wherein one or more double bonds present in the compound are cis or trans, E or Z, a cis/trans mixture, an E/Z mixture, a combination of E and Z geometries, a combination of E and Z geometric mixtures or other geometric isomers thereof.

84. The pharmaceutical composition of any one of claims 63-83, wherein the compound is present in the pharmaceutical composition at a concentration between about 0.005% and about 5%.

85. The pharmaceutical composition of any one of claims 63-84, further comprising a second therapeutic agent.

86. The pharmaceutical composition of any one of claims 63-85, wherein the compound has a lipophilicity as measured by LogP of greater than 3.

87. The pharmaceutical composition of any one of claims 63-86, wherein the compound, or pharmaceutically acceptable salt thereof, is suitable for topical use.

88. A compound having the structure I, or a pharmaceutically acceptable salt thereof, 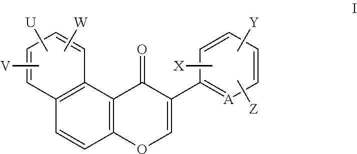
wherein:
each of U, V, W, X, Y, and Z is independently:
H; OH; F; Cl; Br; I; C₁ to C₅ straight chain or branched chain alkyl; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H₂ or a phosphate thereof; PO₂(OCH₃)H or a phosphonate thereof; NO₂; NH₂; NHCH(O); NRCH(O); NHC(O)R; NRC(O)R₁; C(O)NRR₁; C(NH)NRR₁; C(NH)NROH; C(NH)NRNO2; or C(NR)NR1C(NR₂)NR₃R₄;
wherein adjacent substituents U, V and W or X, V and Z may form a saturated or unsaturated 5-membered or 6-membered carbocyclic or heterocyclic ring;
wherein each of R, R₁, R₂, R₃ and R₄ if present is independently:
H; OH; O-Rx; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
wherein Rx, if present, is alkyl, cycloalkyl, alkylcycloalkyl, acyl, ester or thioester;
and wherein A is CH or N.

89. The compound of claim 88, wherein adjacent substituents U, V and W and X, Y and Z may form a saturated or unsaturated 5-membered or 6-membered carbocyclic or heterocyclic ring.

90. The compound of claim 88, wherein:
each of U, V, W, X, Y, and Z is independently:
H; OH; F; Cl; Br; I; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; SO₃H or an ester thereof; CO₂H or an ester thereof; PO₃H2or a phosphate thereof; PO₂(OCH₃)H or a phosphonate thereof; NH₂; NHCH(O); NRCH(O); NHC(O)R; NRC(O)R₁; C(O)NRR₁; C(NH)NRR₁; C(NH)NROH; or C(NR)NR₁C(NR₂)NR₃R₄;
wherein each of R,13₁, R2, R₃ and R₄ if present is independently:
H, OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
and wherein A is CH or N.

91. The compound of claim 89, wherein:
each of U, V, W, X, Y, and Z is independently:
H; OH; F; Cl; Br; I; CH₃; CH₂F; CHF₂; CF₃; O-alkyl; O-cycloalkyl; O-alkylcycloalkyl; OCH₂F; OCHF₂; OCF3; O—(CO)—R; O—(CNH)—R; O—(CNR₁)—R; SO₃H or an ester thereof; CO₂H or an ester thereof; PO₃H₂ or a phosphate thereof; PO₂(OCH₃)H or a phosphonate thereof; NH₂; NHCH(O); NRCH(O); NHC(O)R; NRC(O)R₁; C(O)NRR₁; C(NH)NRR₁; C(NH)NROH; or C(NR)NR₁C(NR₂)NR₃R₄;
wherein each of R, R₁, R₂, R₃ and R₄ if present is independently:
H, OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
and wherein A is CH or N.

92. The compound of claim 90, wherein:
each of U, V, W, X, Y, and Z is independently:
H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;
wherein each of R and RI if present is independently:
H; OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
and wherein A is CH or N.

93. The compound of claim 91, wherein:
each of U, V, W, X, Y, and Z is independently:
H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereofor an ester thereof; NH₂; NHCH(O); NRCH(O); NHC(O)R; NRC(O)R₁; C(O)NRR₁; or C(NH)NRR₁;
wherein each of R and RI if present is independently:
H; OH; optionally substituted alkyl; cycloalkyl; alkylcycloalkyl; heterocycloalkyl; alkylheterocycloalkyl; optionally substituted alkenyl; optionally substituted alkynyl; optionally substituted aryl; optionally substituted alkylaryl; optionally substituted heteroaryl; or optionally substituted alkylheteroaryl;
and wherein A is CH or N.

94. The compound of claim 92, wherein:
each of U, V, W, X, Y, and Z is independently:
H; OH; F; Cl; CH₃; CH₂F; CHF₂; CF₃; O-alkyl; O-cycloalkyl; O-alkylcycloalkyl; OCH₂F; OCHF₂; OCF₃; O—(CO)—R; O—(CNH)—R; O—(CNR₁)—R; CO₂H or an ester thereof; NH₂; NHCH(O); NRCH(O); NHC(O)R; NRC(O)R₁; C(O)NRR₁; or C(NH)NRR₁;
wherein each of R and R₁ if present is independently:
H; OH; optionally substituted alkyl; cycloalkyl; alkylcycloalkyl; heterocycloalkyl; alkylheterocycloalkyl; optionally substituted alkenyl; or optionally substituted alkynyl;
and wherein A is CH or N.

95. The compound of any one of claim 88-94, wherein at least one of U, V and W is H and at least one of X, Y and Z is H.

96. The compound of any one of claims 88, 90, 92 and 94, wherein at least two of U, V and W is H or at least two of X, Y and Z is H.

97. The compound of claim 96, wherein each of U, V and W is H or each of X, Y and Z is H.

98. The compound of claim 97, wherein one of U, V and W is H and each of X, Y and Z is H.

99. The compound of claim 97, wherein one of X, Y and Z is H and each of U, V and W is H.

100. The compound of claim 97, wherein two of U, V and W is H and each of X, Y and Z is H.

101. The compound of claim 97, wherein two of X, Y and Z is H and each of U, V and W is H.

102. The compound of claim 88-101, wherein at least one of U, V, W, X, Y and Z is other than H.

103. The compound of any one of claims 88-97, where the compound is one of the following: 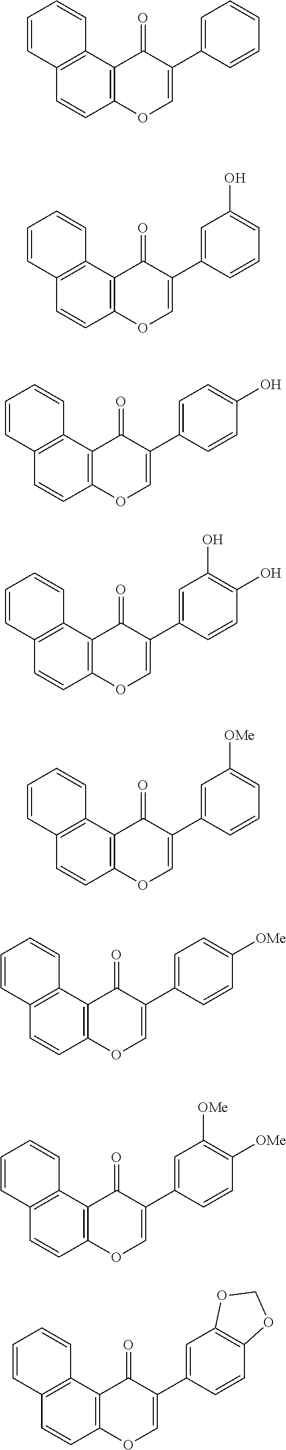 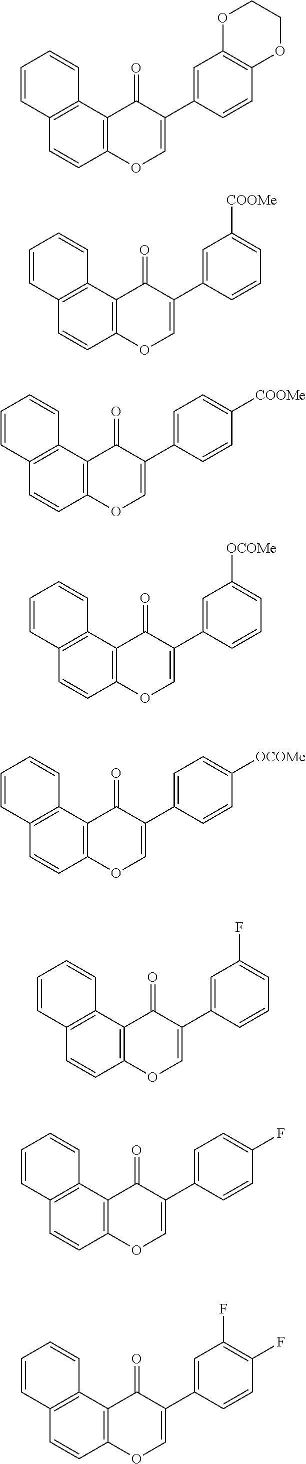 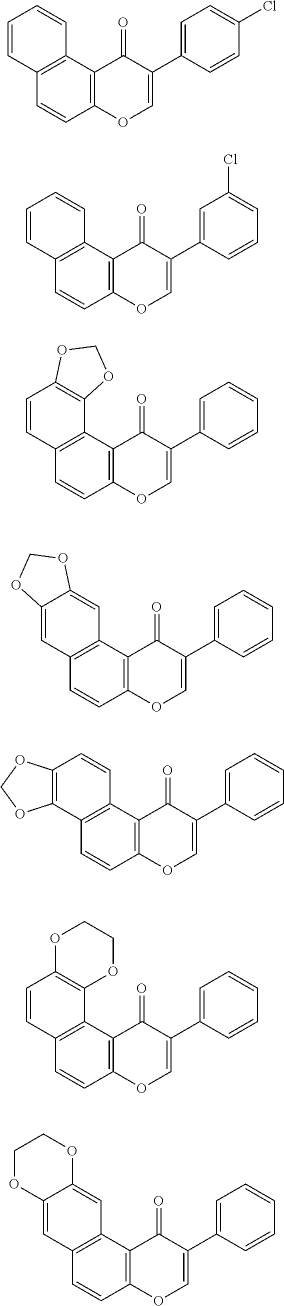 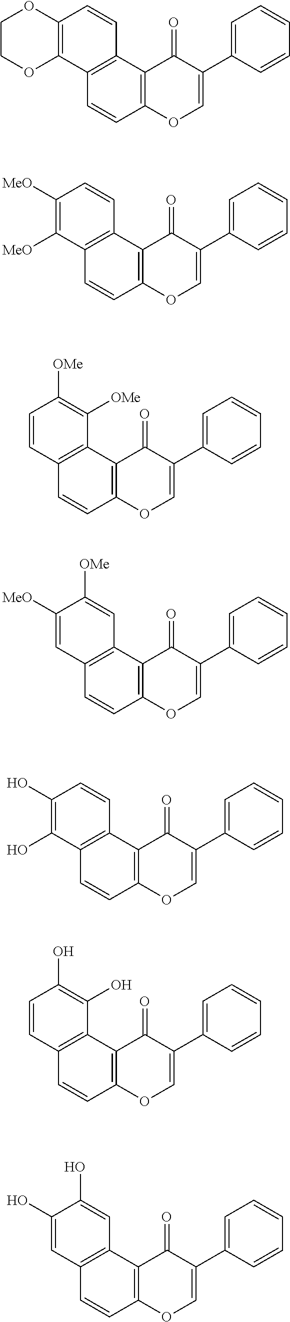
or a pharmaceutically acceptable salt, ester or prodrug form thereof.

104. The compound of any one of claims 88-97, wherein the compound is one of the following: 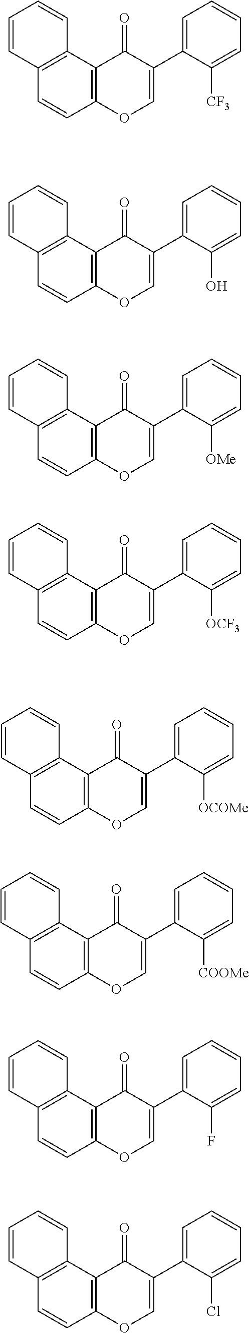 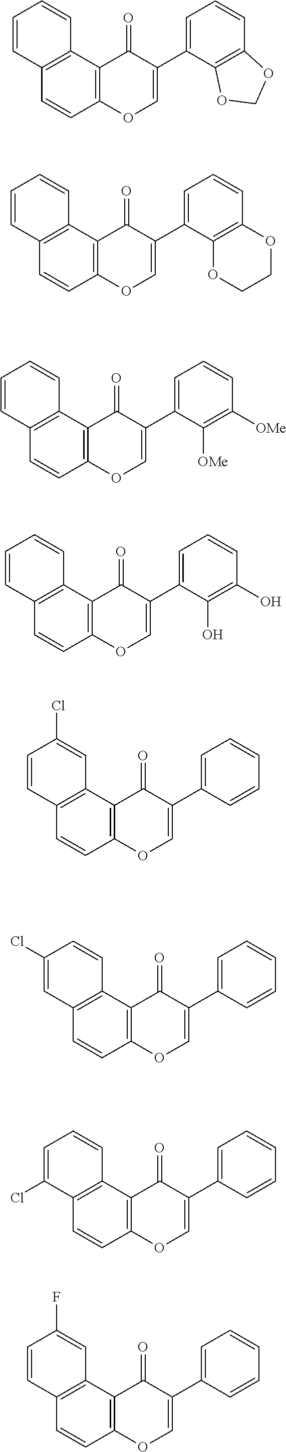 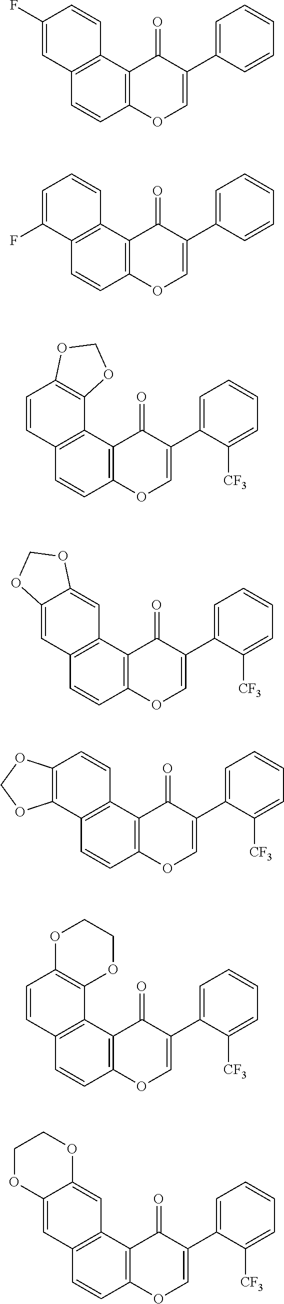 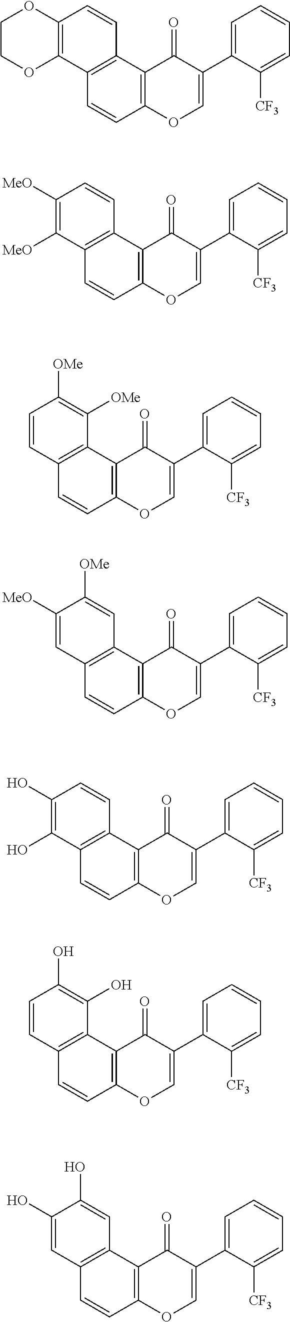
or a pharmaceutically acceptable salt, ester or prodrug form thereof.

105. The compound of any one of claims 8-97, wherein the compound is one of the following: 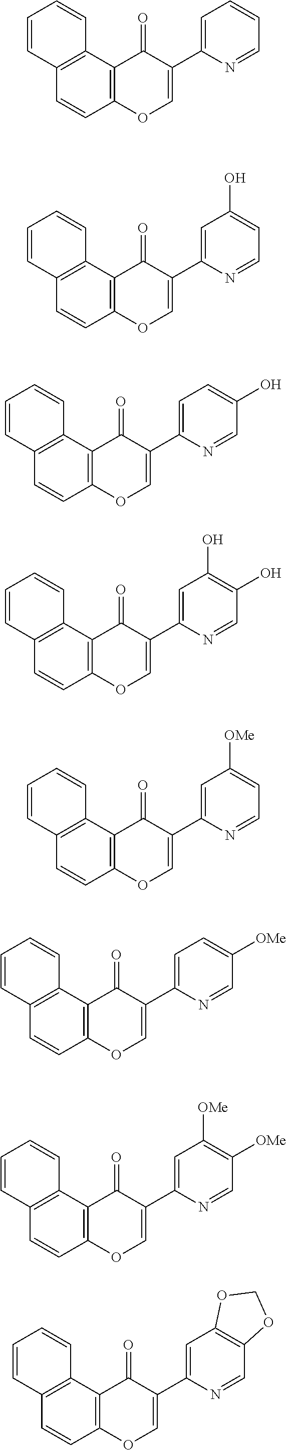 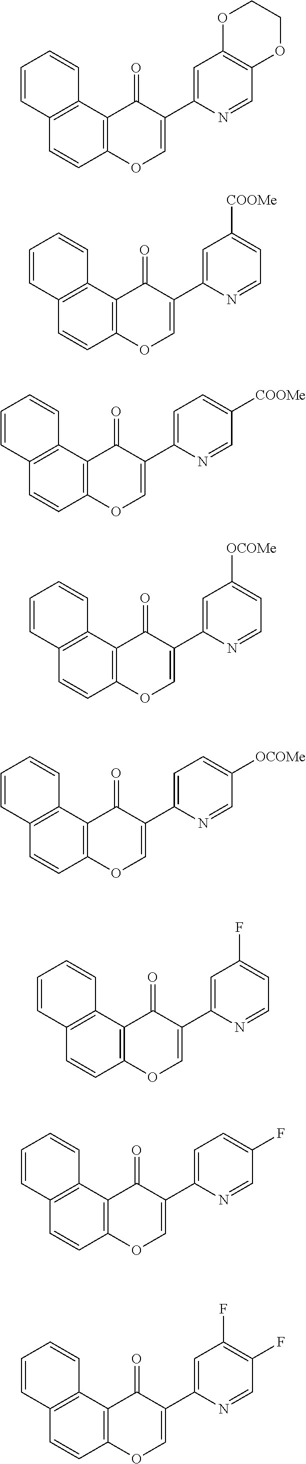 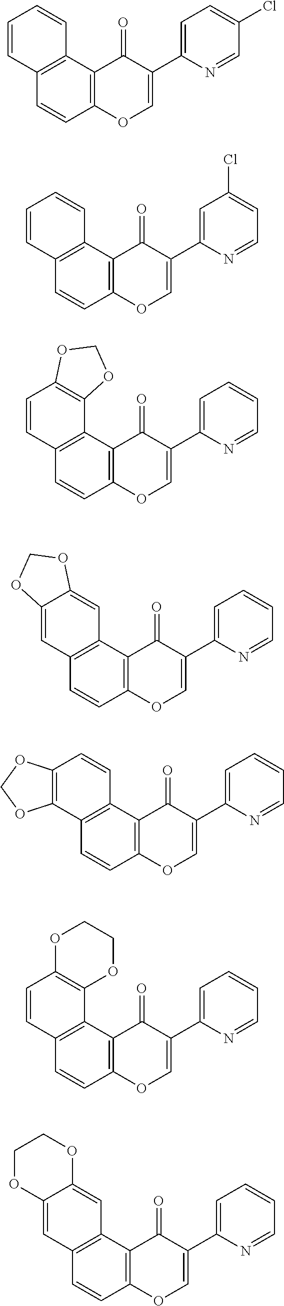 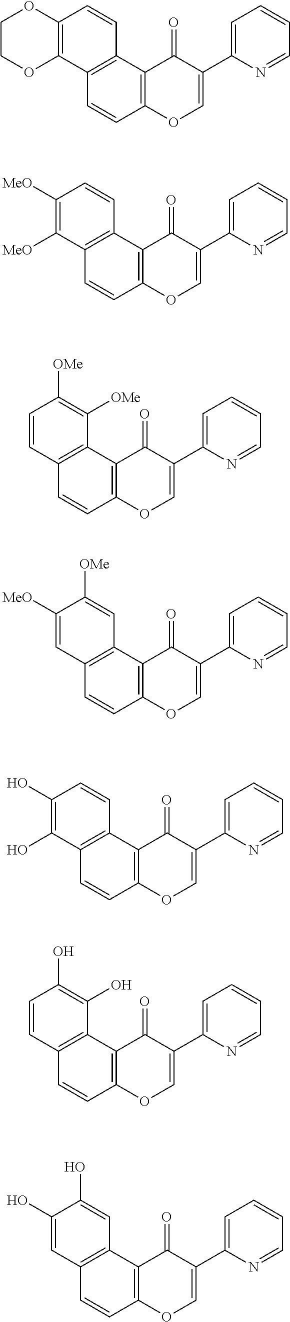
or a pharmaceutically acceptable salt, ester or prodrug form thereof.

106. The compound of any one of claims 88-97, wherein the compound is one of the following: 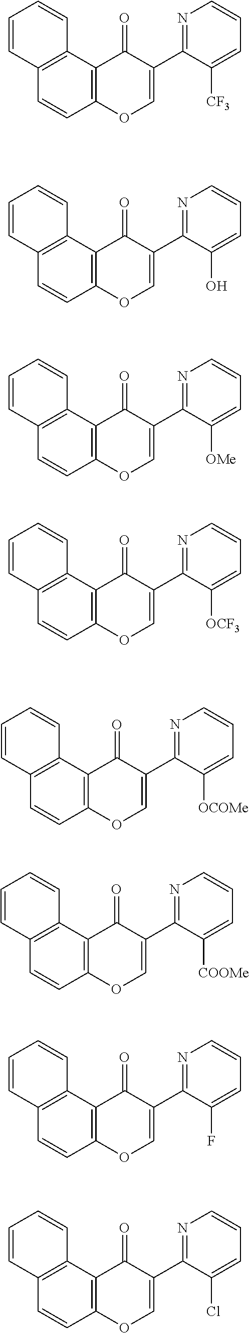 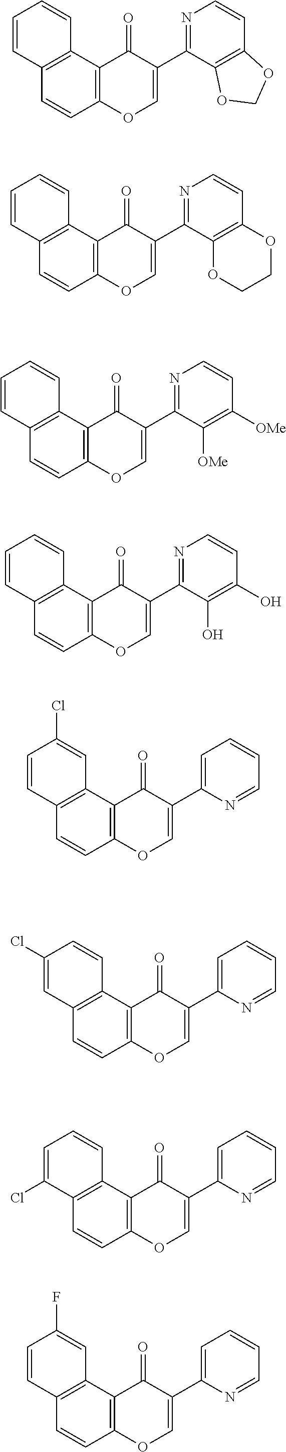 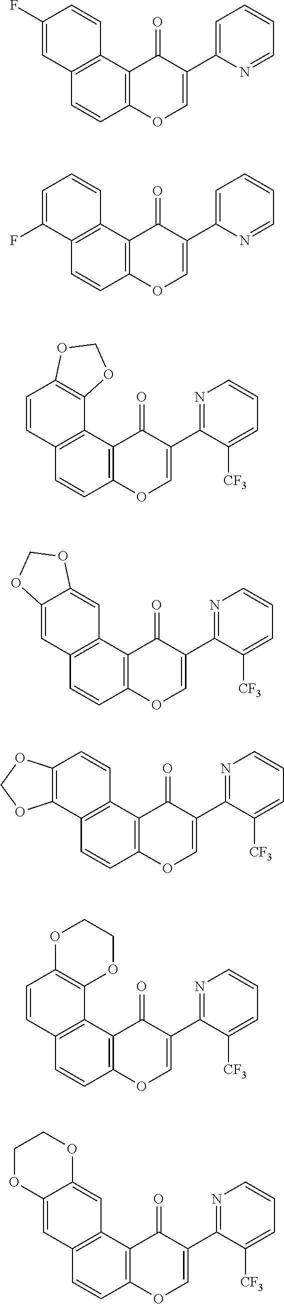 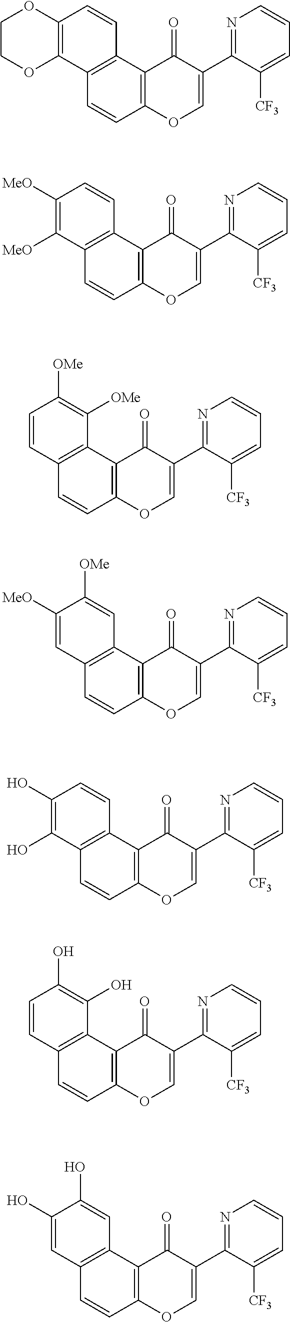
or a pharmaceutically acceptable salt, ester or prodrug form thereof.

107. The compound of any one of claims 88-102, wherein an asymmetric center is present in the compound, and the compound is a racemic mixture, a diastereoisomeric mixture, a single enantiomer, an enantiomeric diastereomer, a meso compound, a pure epimer, ora mixture of epimers thereof.

108. The compound of any one of claims 88-102 and 107, wherein one or more double bonds present in the compound are cis or trans, E or Z, a cis/trans mixture, an E/Z mixture, a combination of E and Z geometries, a combination of E and Z geometric mixtures or other geometric isomers thereof.

109. The compound of any one of claims 88-108, wherein the compound has a lipophilicity as measured by LogP of greater than 3.

110. The compound of any one of claims 88-109, wherein the compound or pharmaceutically acceptable salt thereof is suitable for topical use.
